SEQUENCE LISTING (14 sequences)

SEQ ID NO: 1 - protein, 805 aa, Homo sapiens [sequence omitted]

SEQ ID NO: 2 - protein, 1273 aa, Coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1270 aa, Coronavirus
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 1270 aa, Coronavirus
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 1270 aa, Coronavirus
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQPYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 1273 aa, Coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 1273 aa, Coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - protein, 1273 aa, Coronavirus; identical to SEQ ID NO: 6

SEQ ID NO: 9 - protein, 1273 aa, Coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 1273 aa, Coronavirus
MFVFLVLLPLVSIQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSCMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 1353 aa, Coronavirus
MFLILLISLPTAFAVIGDLKCTSDNINDKDTGPPPISTDTVDVTNGLGTYYVLDRVYLNTTLFLNGYYPTSGSTYRNMALKGSVLLSRLWFKPPFLSDFINGIFAKVKNTKVIKDRVMYSEFPAITIGSTFVNTSYSVVVQPRTINSTQDGDNKLQGLLEVSVCQYNMCEYPQTICHPNLGNHRKELWHLDTGVVSCLYKRNFTYDVNADYLYFHFYQEGGTFYAYFTDTGVVTKFLFNVYLGMALSHYYVMPLTCNSKLTLEYWVTPLTSRQYLLAFNQDGIIFNAVDCMSDFMSEIKCKTQSIAPPTGVYELNGYTVQPIADVYRRKPNLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIEDSVFKPRPAGVLTNHDVVYAQHCFKAPKNFCPCKLNGSCVGSGPGKNNGIGTCPAGTNYLTCDNLCTPDPITFTGTYKCPQTKSLVGIGEHCSGLAVKSDYCGGNSCTCRPQAFLGWSADSCLQGDKCNIFANFILHDVNSGLTCSTDLQKANTDIILGVCVNYDLYGILGQGIFVEVNATYYNSWQNLLYDSNGNLYGFRDYITNRTFMIRSCYSGRVSAAFHANSSEPALLFRNIKCNYVFNNSLTRQLQPINYFDSYLGCVVNAYNSTAISVQTCDLTVGSGYCVDYSKNRRSRGAITTGYRFTNFEPFTVNSVNDSLEPVGGLYEIQIPSEFTIGNMVEFIQTSSPKVTIDCAAFVCGDYAACKSQLVEYGSFCDNINAILTEVNELLDTTQLQVANSLMNGVTLSTKLKDGVNFNVDDINFSPVLGCLGSECSKASSRSAIEDLLFDKVKLSDVGFVEAYNNCTGGAEIRDLICVQSYKGIKVLPPLLSENQISGYTLAATSASLFPPWTAAAGVPFYLNVQYRINGLGVTMDVLSQNQKLIANAFNNALYAIQEGFDATNSALVKIQAVVNANAEALNNLLQQLSNRFGAISASLQEILSRLDALEAEAQIDRLINGRLTALNAYVSQQLSDSTLVKFSAAQAMEKVNECVKSQSSRINFCGNGNHIISLVQNAPYGLYFIHFSYVPTKYVTARVSPGLCIAGDRGIAPKSGYFVNVNNTWMYTGSGYYYPEPITENNVVVMSTCAVNYTKAPYVMLNTSIPNLPDFKEELDQWFKNQTSVAPDLSLDYINVTFLDLQVEMNRLQEAIKVLNQSYINLKDIGTYEYYVKWPWYVWLLICLAGVAMLVLLFFICCCTGCGTSCFKKCGGCCDDYTGYQELVIKTSHDD

SEQ ID NO: 12 - protein, 1356 aa, Coronavirus
MLLIIFILPTTLAVIGDFNCTNFAINDLNTTVPRISEYVVDVSYGLGTYYILDRVYLNTTILFTGYFPKSGANFRDLSLKGTTYLSTLWYQKPFLSDFNNGIFSRVKNTKLYVNKTLYSEFSTIVIGSVFINNSYTIVVQPHNGVLEITACQYTMCEYPHTICKSKGSSRNESWHFDKSEPLCLFKKNFTYNVSTDWLYFHFYQERGTFYAYYADSGMPTTFLFSLYLGTLLSHYYVLPLTCNAISSNTDNETLQYWVTPLSKRQYLLKFDNRGVITNAVDCSSSFFSEIQCKTKSLLPNTGVYDLSGFTVKPVATVHRRIPDLPDCDIDKWLNNFNVPSPLNWERKIFSNCNFNLSTLLRLVHTDSFSCNNFDESKIYGSCFKSIVLDKFAIPNSRRSDLQLGSSGFLQSSNYKIDTTSSSCQLYYSLPAINVTINNYNPSSWNRRYGFNNFNLSSHSVVYSRYCFSVNNTFCPCAKPSFASSCKSHKPPSASCPIGTNYRSCESTTVLDHTDWCRCSCLPDPITAYDPRSCSQKKSLVGVGEHCAGFGVDEEKCGVLDGSYNVSCLCSTDAFLGWSYDTCVSNNRCNIFSNFILNGINSGTTCSNDLLQPNTEVFTDVCVDYDLYGITGQGIFKEVSAVYYNSWQNLLYDSNGNIIGFKDFVTNKTYNIFPCYAGRVSAAFHQNASSLALLYRNLKCSYVLNNISLTTQPYFDSYLGCVFNADNLTDYSVSSCALRMGSGFCVDYNSPSSSSSRRKRRSISASYRFVTFEPFNVSFVNDSIESVGGLYEIKIPTNFTIVGQEEFIQTNSPKVTIDCSLFVCSNYAACHDLLSEYGTFCDNINSILDEVNGLLDTTQLHVADTLMQGVTLSSNLNTNLHFDVDNINFKSLVGCLGPHCGSSSRSFFEDLLFDKVKLSDVGFVEAYNNCTGGSEIRDLLCVQSFNGIKVLPPILSESQISGYTTAATVAAMFPPWSAAAGIPFSLNVQYRINGLGVTMDVLNKNQKLIATAFNNALLSIQNGFSATNSALAKIQSVVNSNAQALNSLLQQLFNKFGAISSSLQEILSRLDALEAQVQIDRLINGRLTALNAYVSQQLSDISLVKFGAALAMEKVNECVKSQSPRINFCGNGNHILSLVQNAPYGLLFMHFSYKPISFKTVLVSPGLCISGDVGIAPKQGYFIKHNDHWMFTGSSYYYPEPISDKNVVFMNTCSVNFTKAPLVYLNHSVPKLSDFESELSHWFKNQTSIAPNLTLNLHTINATFLDLYYEMNLIQESIKSLNNSYINLKDIGTYEMYVKWPWYVWLLISFSFIIFLVLLFFICCCTGCGSACFSKCHNCCDEYGGHHDFVIKTSHDD

SEQ ID NO: 13 - protein, 1356 aa, Coronavirus
MKLFLILLVLPLASCFFTCNSNANLSMLQLGVPDNSSTIVTGLLPTHWFCANQSTSVYSANGFFYIDVGNHRSAFALHTGYYDANQYYIYVTNEIGLNASVTLKICKFSRNTTFDFLSNASSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNGSTLVDGVSRLYQPLRLTCLWPVPGLKSSTGFVYFNATGSDVNCNGYQHNSVVDVMRYNLNFSANSLDNLKSGVIVFKTLQYDVLFYCSNSSSGVLDTTIPFGPSSQPYYCFINSTINTTHVSTFVGILPPTVREIVVARTGQFYINGFKYFDLGFIEAVNFNVTTASATDFWTVAFATFVDVLVNVSATNIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVEVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIRSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDAITQTAEAIHTVTIALNKIQDVVNQQGSALNHLTSQLRHNFQAISNSIQAIYDRLDSIQADQQVDRLITGRLAALNAFVSQVLNKYTEVRGSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTTVELQGLIDQINSTYVDLKLLNRFENYIKWPWWVWLIISVVFVVLLSLLVFCCLSTGCCGCCNCLTSSMRGCCDCGSTKLPYYEFEKVHVQ

SEQ ID NO: 14 - protein, 1173 aa, Coronavirus
MFVLLVAYALLHIAGCQTTNGLNTSYSVCNGCVGYSENVFAVESGGYIPSDFAFNNWFLLTNTSSVVDGVVRSFQPLLLNCLWSVSGLRFTTGFVYFNGTGRGDCKGFSSDVLSDVIRYNLNFEENLRRGTILFKTSYGVVVFYCTNNTLVSGDAHIPFGTVLGNFYCFVNTTIGNETTSAFVGALPKTVREFVISRTGHFYINGYRYFTLGNVEAVNFNVTTAETTDFCTVALASYADVLVNVSQTSIANIIYCNSVINRLRCDQLSFDVPDGFYSTSPIQSVELPVSIVSLPVYHKHTFIVLYVDFKPQSGGGKCFNCYPAGVNITLANFNETKGPLCVDTSHFTTKYVAVYANVGRWSASINTGNCPFSFGKVNNFVKFGSVCFSLKDIPGGCAMPIVANWAYSKYYTIGSLYVSWSDGDGITGVPQPVEGVSSFMNVTLDKCTKYNIYDVSGVGVIRVSNDTFLNGITYTSTSGNLLGFKDVTKGTIYSITPCNPPDQLVVYQQAVVGAMLSENFTSYGFSNVVELPKFFYASNGTYNCTDAVLTYSSFGVCADGSIIAVQPRNVSYDSVSAIVTANLSIPSNWTTSVQVEYLQITSTPIVVDCSTYVCNGNVRCVELLKQYTSACKTIEDALRNSARLESADVSEMLTFDKKAFTLANVSSFGDYNLSSVIPSLPTSGSRVAGRSAIEDILFSKLVTSGLGTVDADYKKCTKGLSIADLACAQYYNGIMVLPGVADAERMAMYTGSLIGGIALGGLTSAVSIPFSLAIQARLNYVALQTDVLQENQKILAASFNKAMTNIVDAFTGVNDAITQTSQALQTVATALNKIQDVVNQQGNSLNHLTSQLRQNFQAISSSIQAIYDRLDTIQADQQVDRLITGRLAALNVFVSHTLTKYTEVRASRQLAQQKVNECVKSQSKRYGFCGNGTHIFSIVNAAPEGLVFLHTVLLPTQYKDVEAWSGLCVDGTNGYVLRQPNLALYKEGNYYRITSRIMFEPRIPTMADFVQIENCNVTFVNISRSELQTIVPEYIDVNKTLQELSYKLPNYTVPDLVVEQYNQTILNLTSEISTLENKSAELNYTVQKLQTLIDNINSTLVDLKWLNRVETYIKWPWWVWLCISVVLIFVVSMLLLCCCSTGCCGFFSCFASSIRGCCESTKLPYYDVEKIHIQ

(19) United States
(12) Patent Application Publication
(10) Pub. No.: US 2025/0177560 A1
(43) Pub. Date: Jun. 5, 2025

(54) FUNCTIONALIZED NANOPARTICLES FOR THE CONTAINMENT AND CLEARANCE OF PATHOGENS

(71) Applicant: The University of Chicago, Chicago, IL (US)

(72) Inventors: Jun Huang, Chicago, IL (US); Jillian Rosenberg, Chicago, IL (US); Min Chen, Chicago, IL (US)

(73) Assignee: The University of Chicago, Chicago, IL (US)

(21) Appl. No.: 18/274,444

(22) PCT Filed: Jan. 27, 2022
(86) PCT No.: PCT/US2022/014163

§ 371 Date: Jul. 26, 2023

Related U.S. Application Data
(60) Provisional application No. 63/142,320, filed on Jan. 27, 2021.

(51) Int. Cl. A61K 47/69; A61K 9/00; A61K 39/00; A61K 47/54; A61P 31/04; C07K 16/10

(52) U.S. Cl. CPC A61K 47/6937; A61K 9/0019; A61K 47/544; A61P 31/04; C07K 16/1003; A61K 2039/505

(57) ABSTRACT

Functionalized nanoparticles for inhibiting or preventing pathogen infections (e.g., viral or bacterial infections, such as coronavirus infections) are described. The nanoparticles comprise a biodegradable polymer core and a lipid coating layer that is functionalized with a pathogen-binding receptor (e.g., an angiotensin-converting enzyme 2 (ACE2) receptor protein) and/or a pathogen-binding antibody or an antigen-binding fragment thereof (e.g., a virus-binding antibody or an antigen-binding fragment thereof). The nanoparticles are further functionalized by a phagocyte-specific ligand, e.g., a phosphatidylserine-containing lipid included in the lipid coating layer, to promote clearance of nanoparticle-bound pathogen. Methods of using the nanoparticles to treat or prevent pathogen infections (e.g., coronavirus infections) are also described.

### RELATED APPLICATIONS

The presently disclosed subject matter claims the benefit of U.S. Provisional Patent Application Ser. No. 63/142,320, filed Jan. 27, 2021, the disclosure of which is incorporated herein by reference in its entirety.

### STATEMENT OF GOVERNMENT INTEREST

This invention was made with government support under grant number 1653782 awarded by the National Science Foundation and grant numbers AI144245 and DK122394 awarded by the National Institutes of Health. The government has certain rights in the invention.

### REFERENCE TO SEQUENCE LISTING SUBMITTED ELECTRONICALLY

The content of the electronically submitted sequence listing in ASCII text file (Name: 3072-22_PCT_ST25.txt; Size: 149 kilobytes; and Date of Creation: Jan. 27, 2022) filed with the application is incorporated herein by reference in its entirety.

### TECHNICAL FIELD

The presently disclosed subject matter relates to functionalized nanoparticles for inhibiting pathogen infection, e.g., viral or bacterial infection, including severe acute respiratory syndrome coronavirus 2 (SARS-CoV-2) infection or another coronavirus infection. The nanoparticles are functionalized with groups that mimic pathogen target cells and/or that bind to pathogen proteins, as well as groups that can target the functionalized nanoparticles for macrophage engulfment and clearance. The presently disclosed subject matter further relates to the use of the functionalized nanoparticles in treating or preventing pathogen infection.

ABBREVIATIONS

| | |
|---|---|
| ° C.= | degrees Celsius |
| %= | percentage |
| μg= | microgram |
| μl or μL= | microliter |
| μM= | micromolar |
| ACE2= | angiotensin-converting enzyme 2 |
| CoV= | coronavirus |
| DOPA= | dioleoyl-sn-glycero-3-phosphate |
| DOPC= | 1,2-dioleoyl-sn-glycero-3-phosphate sodium salt |
| DSPE-PEG₂ₖ= | 1,2-distearoyl-sn-glycero-3-phosphoethanolamine- |
| | N-[amino(polyethylene glycol)₂₀₀₀] |
| EtOH= | ethanol |
| EVLP= | ex vivo lung perfusion |
| g= | gram |
| h= | hour |
| IC₅₀= | fifty percent inhibitory concentration |
| kg= | kilogram |
| M= | molar |
| mg= | milligram |
| min= | minute |
| mL= | milliliter |
| mm= | millimeter |
| mM= | millimolar |
| mmol= | millimole |
| mV= | millivolts |
| nm= | nanometer |
| NMR= | nuclear magnetic resonance |
| PBS= | phosphate buffered saline |
| PEG= | polyethylene glycol |
| PLA= | polylactic acid |
| PS= | phosphatidylserine |
| PVP= | polyvinylpyrrolidone |
| SARS= | severe acute respiratory syndrome |
| SEM= | scanning electron microscopy |
| THF= | tetrahydrofuran |

### BACKGROUND

Severe acute respiratory syndrome coronavirus 2 (SARS-CoV-2) caused the global pandemic of coronavirus disease 2019 (COVID-19). As of Dec. 9, 2020, SARS-CoV-2 has spread to over 180 countries and has resulted in more than 68.6 million infections and over 1.56 million deaths globally¹. Remdesivir has been approved by the Food and Drug Administration (FDA) to treat severe COVID-19,⁴² despite its inconsistent clinical benefits and various reported adverse effects^{43,44}. Transfusion of convalescent plasma from recovered patients has shown clinical benefits in some COVID-19 patients.⁴⁵ However, this approach is challenged by the limited availability of donor plasma and appropriate medical facilities.⁴⁶ Simultaneously, significant efforts have been devoted to the development of vaccines, neutralizing antibodies, and other drugs for the prevention and treatment of COVID-19. However, there is an ongoing need for additional compositions and methods for treating or preventing SARS-CoV-2 and other pathogen infections safely and effectively.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter provides a nanoparticle comprising: (a) a core comprising a biocompatible polymer; and (b) an outer layer encapsulating said core, wherein the outer layer comprises one or more lipids and/or one or more proteins, and wherein an outer surface of the outer layer comprises: (c) a pathogen-binding receptor and/or a pathogen-binding antibody or an antigen-binding fragment thereof; and (d) a phagocyte-specific ligand. In some embodiments, the biocompatible polymer comprises a biodegradable polymer, optionally wherein the biodegradable polymer comprises a polyester, a polyether, or a polyamide, further optionally wherein the biodegradable polymer comprises polylactic acid (PLA), poly(lactic-co-glycolic acid) (PLGA), a PLGA-PLA copolymer, polypyrrole, or a mixture thereof. In some embodiments, the core has a diameter between about 200 and about 1200 nanometers, optionally about 500 nanometers.

In some embodiments, (c) comprises a virus-binding receptor and/or a virus-binding antibody or an antigen-binding fragment thereof, optionally a virus-neutralizing antibody or an antigen-binding fragment thereof. In some embodiments, (c) comprises an angiotensin converting enzyme 2 (ACE2) receptor protein, optionally wherein the ACE2 receptor protein is a streptavidin-modified ACE2 receptor protein, the outer layer comprises a biotin-modified lipid, and wherein the streptavidin-modified ACE2 receptor is attached to the surface of the outer layer via biotin-streptavidin non-covalent binding interactions. In some embodiments, (c) comprises a virus-neutralizing antibody or an antigen-binding fragment thereof, optionally wherein the virus-neutralizing antibody or antigen-binding fragment thereof is covalently attached to a lipid in the outer layer via reaction with an active ester-functionalized lipid.

In some embodiments, the virus-neutralizing antibody or the antigen-binding fragment thereof is a coronavirus-neutralizing antibody or antigen-binding fragment thereof, optionally a severe acute respiratory syndrome coronavirus 2 (SARS-CoV-2)-neutralizing antibody or an antigen-binding fragment thereof. In some embodiments, the virus-neutralizing antibody or the antigen-binding fragment thereof is an antibody or antigen-binding fragment thereof that binds to a SARS-CoV-2 spike protein.

In some embodiments, the outer layer comprises one or more phosphatidylserine lipid selected from the group comprising 1,2-dioleoyl-sn-glycero-3-phospho-L-serine (DOPS), 1,2-distearoyl-sn-glycero-3-phosphatidylserine (DSPS), 1;1-palmitoyl-2-oleoyl-sn-glycero-3-phospho-L-serine (POPS), 1,2-dipalmitoyl-sn-glycero-3-phosphoserine (DPPS), and 1,2-ditetradecanoyl-sn-glycero-3-phospho-L-serine (DMPS); optionally about 15% of the one or more phosphatidylserine lipid; and wherein the phagocyte-specific ligand (d) comprises a phosphoserine moiety from the one or more phosphatidylserine lipid. In some embodiments, the outer layer comprises one or more of DOPS, DSPS, POPS, DPPS, and DMPS, optionally about 15% of the one or more DOPS, DSPS, POPS, DPPS, and DMPS, and wherein the phagocyte-specific ligand (d) comprises a moiety targeting a phagocytic cell, optionally wherein the phagocytic cell is selected from the group consisting of a macrophage, a dendritic cell, a neutrophil, a monocyte, and a mast cell.

In some embodiments, the outer layer comprises one or more of the group consisting of 1,2-distearoyl-sn-glycero-3-phosphocholine (DSPC), cholesterol, and a poly(ethylene glycol) (PEG)-modified lipid, optionally 1,2-distearoyl-sn-glycero-3-phosphoethanolamine (DSPE)-mPEG₂₀₀₀. In some embodiments, the core further comprises perfluorooctyl bromide (PFOB).

In some embodiments, the presently disclosed subject matter provides a pharmaceutical formulation comprising a nanoparticle comprising: (a) a core comprising a biocompatible polymer; and (b) an outer layer encapsulating said core, wherein the outer layer comprises one or more lipids and/or one or more proteins, and wherein an outer surface of the outer layer comprises: (c) a pathogen-binding receptor and/or a pathogen-binding antibody or an antigen-binding fragment thereof; and (d) a phagocyte-specific ligand; and a pharmaceutically acceptable carrier.

In some embodiments, the presently disclosed subject matter provides a method of treating or preventing a pathogen infection in a subject in need of treatment or prevention thereof, wherein the method comprises administering to said subject a nanoparticle comprising: (a) a core comprising a biocompatible polymer; and (b) an outer layer encapsulating said core, wherein the outer layer comprises one or more lipids and/or one or more proteins, and wherein an outer surface of the outer layer comprises: (c) a pathogen-binding receptor and/or a pathogen-binding antibody or an antigen-binding fragment thereof; and (d) a phagocyte-specific ligand; or a pharmaceutical formulation thereof. In some embodiments, the pathogen infection is a viral infection. In some embodiments, the viral infection is a coronavirus infection, optionally wherein the coronavirus infection is a SARS-CoV-2 infection.

In some embodiments, the administering is performed intranasally. In some embodiments, the administering is performed orally, intravenously, subcutaneously, intramuscularly, or via ocular administration. In some embodiments, the subject is a mammal, optionally a human.

In some embodiments, the presently disclosed subject matter provides a nanoparticle for use in treating or preventing a pathogenic infection, optionally a viral infection, further optionally a SARS-CoV-2 infection, wherein the nanoparticle comprises: (a) a core comprising a biocompatible polymer; and (b) an outer layer encapsulating said core, wherein the outer layer comprises one or more lipids and/or one or more proteins, and wherein an outer surface of the outer layer comprises: (c) a pathogen-binding receptor and/or a pathogen-binding antibody or an antigen-binding fragment thereof; and (d) a phagocyte-specific ligand.

Accordingly, it is an object of the presently disclosed subject matter to provide nanoparticles comprising a biocompatible polymer core, an outer lipid layer, and a pathogen-binding receptor and/or a pathogen-binding antibody, and a phagocyte-specific ligand, as well as related pharmaceutical formulations and methods of treating or preventing a pathogen infection.

An object of the presently disclosed subject matter having been stated hereinabove, and which is achieved in whole or in part by the presently disclosed subject matter, other objects will become evident as the description proceeds hereinbelow.

### BRIEF DESCRIPTION OF THE DRAWINGS

FIGS. 1A-1F: Schematic design, synthesis, and characterization of functionalized nanoparticles of the presently disclosed subject matter for use in treating or preventing Severe Acute Respiratory Syndrome coronavirus 2 (SARS-CoV-2). FIG. 1A is a schematic illustration showing a nanoparticle with a polymeric core coated with a lipid bilayer functionalized with angiotensin-converting enzyme 2 (ACE2) protein/virus neutralizing antibody and including a phosphatidylserine interacting with a SARS-CoV-2 virion. Following intratracheal administration (center and right), the functionalized nanoparticle efficiently accumulates and traps SARS-CoV-2 virions in the lung tissue forming virus-nanoparticle complexes, which can be cleared by macrophages via phagocytosis, thereby blocking viral cell-entry. FIGS. 1B and 1C are graphs showing (FIG. 1B) the average diameter (in nanometers (nm)) measured by dynamic light scattering (DLS) and (FIG. 1C) zeta-potential measurements (in millivolts (mV)) during different stages of nanoparticle preparation (i.e., where PLA=polylactic acid core; DOPS=lipid layer including biotinylated lipid; SA=streptavidin; ACE2=biotinylated ACE2). FIG. 1D is a series of fluorescent images of the prepared functionalized nanoparticles with a polylactic acid (PLA) polymeric core (indicated by fluorescent dye in image on left) and ACE2 (indicated by fluorescently labelled anti-ACE2 antibody in image in middle). Image on right is an image where fluorescent signals for both the core and ACE2 are shown. Scale bar represents 5 micrometers (μm). Dotted lines represent displayed plot profile below each image. FIGS. 1E and 1F are scanning electron microscopy (SEM) images of functionalized nanoparticles alone (FIG. 1E) or with SARS-CoV-2 pseudovirus (FIG. 1F), where pseudovirus is indicated by the lighter grey dots on the nanoparticle surface. To better visualize the selectivity for viral binding, larger nanoparticles were imaged. Scale bar represents 300 nm.

FIGS. 2A-2H: Phagocytosis of functionalized nanoparticles of the presently disclosed subject matter by macrophages. Functionalized nanoparticles of the presently disclosed subject matter of varying size (200, 500, and 1200 nanometer (nm) average diameter) and phosphatidylserine (PS) densities were incubated with differentiated THP-1 (dTHP-1) macrophages. FIGS. 2A and 2B are graphs showing the percent uptake (FIG. 2A) and mean fluorescent intensity (MFI, FIG. 2B) quantification of flow cytometry measurements of the internalization of functionalized nanoparticles with varying diameters after 0 hours, 24 hours and 48 hours. Data for untreated macrophages is shown as a negative control. FIGS. 2C and 2D are graphs showing the percent uptake (FIG. 2C) and MFI (FIG. 2D) quantification of flow cytometry measurements of the internalization of functionalized nanoparticles of the presently disclosed subject matter with varying phosphatidylserine (PS) ratios (0%, 5%, 10%, or 15% PS) after 0, 2, 4, 6, 24, or 48 hours. Data are shown as mean±standard deviation (SD); unpaired t tests were conducted from three replicates. FIGS. 2E and 2F are series of lattice light-sheet microscopy images of macrophages (Wheat Germ Agglutinin (WGA)-CF488) phagocytosing functionalized nanoparticles of the presently disclosed subject matter with varying PS ratios (0%, 10%, or 15%) after 24 hours (FIG. 2E) or 48 hours (FIG. 2F). Images of untreated macrophages are shown in the left-most image of each figure. Scale bar represents 5 micrometers (μm). FIG. 2G is a group of confocal microscopy images of dTHP-1 cells stained for DAPI (upper left image), lysosomes (LAMP-1-AF488, lower left image), and endosomes (EEA1-AF594, upper middle image) incubated with angiotensin-converting enzyme 2 (ACE2)-functionalized nanoparticles (Nanotrap-ACE2, DiD, lower middle image) for 6 hours. Dotted line in the image on the right indicates line scan analyzed in FIG. 7. Scale bar represents 10 micrometers (μm). FIG. 2H is a graph based on data from a study where viral particles labeled with lipophilic dye DiO were incubated with functionalized nanoparticles of the presently disclosed subject matter (labeled with dye DiD) at 37° C. for various time points or dTHP-1 macrophages were incubated with functionalized nanoparticles at 37° C. for various time points. Percent binding was measured by flow cytometry. Double-positive events were gated and the mean fluorescence intensity of DiO was measured over time until saturation was reached. Data were fitted with a curve and t_{1/2} was extrapolated at the time at which 50% of nanoparticles were bound or engulfed.

FIGS. 3A-3D: Inhibition of severe acute respiratory syndrome coronavirus 2 (SARS-CoV-2) viral infection of host cells. FIG. 3A is a pair of graphs of HEK293T-ACE2 cells treated with SARS-CoV-2 spike pseudotyped lentivirus (left) or vesicular stomatitis virus (VSV, right) and angiotensin-converting enzyme 2 (ACE2)-functionalized nanoparticles (Nanotrap-ACE2), Anti-SARS-CoV-2 antibody-functionalized nanoparticles (Nanotrap-Antibody), or nanoparticles without antibody or ACE2 functionalization (Nanotrap-Blank) for 72 and 24 hours, respectively. Data are presented as mean±standard deviation (SD) and fitted with a two-phase decay model. FIG. 3B is a pair of graphs of HEK293T-ACE2 cells were treated with SARS-CoV-2 spike pseudotyped lentivirus (left) or VSV (right) and Nanotrap-ACE2 or soluble angiotensin-converting enzyme 2 (ACE2) for 72 and 24 hours, respectively. Data are presented as mean±SD and fitted with a trend curve. For both SARS-CoV-2 spike pseudotyped lentivirus (left) and VSV (right), the Nanotrap-ACE2 and soluble ACE2 curves differ with p<0.0001, as tested by sum-of-squares F tests. FIG. 3C is a series of confocal microscopy images of pseudotyped VSV infection (GFP) in differentiated THP-1 (dTHP1) macrophages (WGA) and A549 epithelial cells (DAPI) with or without nanoparticles of the presently disclosed subject matter. Scale bars represent 100 micrometers (km), with inset scale bars representing 40 μm. MΦ: macrophages; V: virus; E: epithelial cells; NT: Nanotrap-ACE2. FIG. 3D is a graph showing quantification of data (percent green fluorescent protein (GFP+)) from FIG. 3C. Data are shown as mean±SD; unpaired t tests were conducted from three independent experiments.

FIGS. 4A-4E: Murine in vivo biosafety profile of treatment with functionalized nanoparticles of the presently disclosed subject matter. Wild-type (WT) B6 mice were treated intratracheally with 10 milligrams per kilogram (mg/kg) of angiotensin-converting enzyme 2 (ACE2)-functionalized nanoparticles (Nanotrap-ACE2) or phosphate buffered saline (PBS) for 72 hours (n=4, 2 male+2 female). FIGS. 4A and 4B are representative fluorescent images of lung tissues of functionalized nanoparticle-treated mice (FIG. 4A) or PBS-treated mice (FIG. 4B) 72 hours post-intratracheal administration. Scale bars represent 250 micrometers (μm); inset scale bars represent 50 m. FIG. 4C is a series of representative hematoxylin and eosin (H&E) staining images of major organs sections, including heart, liver, spleen, lung and kidney of functionalized nanoparticle-treated mice (Nanotrap, bottom row) or PBS-treated mice (top row). Scale bars represent 500 m. FIG. 4D is a graph of blood cell counts (cell count per microliter (μL)) of white blood cells (WBC), red blood cells (RBC) and platelets (PLT) 72 hours after functionalized nanoparticle (Nanotrap) or PBS treatment. Data are shown as mean±standard deviation (SD); unpaired t tests were conducted from 4 replicates. FIG. 4E is a graph of comprehensive blood chemistry panels comparing functionalized nanoparticle (Nanotrap)- and PBS-treated mice. ALP: alkaline phosphatase; ALT: alanine aminotransferase; AMYL: amylase; BUN: urea nitrogen; CA: calcium; Chol: cholesterol; GLU: glucose; TBIL: total bilirubin; CTP: total protein.

FIGS. 5A-5E: Ex vivo human lung perfusion system for evaluating the neutralizing ability of exemplary functionalized nanoparticles (i.e., anti-viral antibody-functionalized (Nanotrap-Antibody) nanoparticles). FIG. 5A is a pair of images of a laboratory system for studying human ex vivo lung perfusion (EVLP). Arrows in the image on the right indicate untreated (right upper lung), virus only (right middle lung), and virus+functionalized nanoparticle (NT) (lingula) regions. FIG. 5B is a graph showing the quantification of luciferase expression (in relative luminescence units (RLU)) in EVLP samples 8 hours post-infection in the three regions indicated in the right panel of FIG. 5A. Data are shown as mean±standard deviation (SD). FIG. 5C is a series of hematoxylin and eosin (H&E) staining images of EVLP samples. Scale bars represent 500 micrometers (μm); inset scale bars represent 200 m. FIG. 5D is a graph showing quantification of luciferase expression in in vitro primary human cells 48 hours post-infection. Data are shown as mean±SD. FIG. 5E is a graph of infectivity (measured as a percentage) in Vero E6 cells treated with authentic Severe Acute Respiratory Syndrome coronavirus 2 (SARS-CoV-2) and an nanoparticle without antibody or other virus targeting functionalization (Nanotrap-Blank) or an anti-virus antibody-functionalized nanoparticle (Nanotrap-Antibody). Data are shown as mean±SD fitted with a trend curve.

FIGS. 6A-6H: Schematic design, synthesis, and characterization of functionalized nanoparticles of the presently disclosed subject matter for treating or preventing severe acute respiratory syndrome coronavirus 2 (SARS-CoV-2) infection. FIG. 6A is a series of graphs of the dynamic light scattering (DLS) measurements of functionalized nanoparticles designed with 200 nanometer (nm, left), 500 nm (middle), and 1200 nm (right) average diameter core sizes. FIG. 6B is a scanning electron microscopy (SEM) image of a dispersion of 500 nm diameter nanoparticles without virus. Scale bar represents 1 micrometer (μm). FIG. 6C is a transmission electron microscopy (TEM) image of a nanoparticle of the presently disclosed subject matter showing the core-shell structure. Scale bar represents 250 nm. FIG. 6D is a series of fluorescent images of functionalized nanoparticles with a polylactic acid (PLA) polymeric core (DiD labeled) and angiotensin-converting enzyme 2 (ACE2) (anti-ACE2-AF488-labeled) that had been stored at −20 degrees Celsius (° C.) for six months. Scale bar represents 5 μm. Dotted lines represent displayed plot profile below each image. FIG. 6E is graph showing DLS measurements comparing freshly made PLA core or functionalized nanoparticles with PLA core or functionalized nanoparticles that had been lyophilized, sealed, and stored at −20° C. for six months. FIG. 6F is a graph showing histograms displaying phycoerythrin (PE)-labelled beads, antibody-functionalized nanoparticles (Nanotrap-Antibody) labeled with anti-immunoglobulin G (IgG)-PE, and ACE2-functionalized nanoparticles (Nanotrap-ACE2) labeled with anti-ACE2-PE. FIG. 6G is a graph showing average surface density of the functionalized antibodies calculated using the PE-labelled beads. The average surface densities are calculated as (3.59±0.43)×10⁵ ACE2 molecules per Nanotrap-ACE2, and (2.47±0.21)×10⁴ neutralizing antibodies per Nanotrap-Antibody. FIG. 6H is a schematic diagram showing orientation of antibodies on the surface of the functionalized nanoparticles resultant of N-hydroxysuccinimide (NHS)-amine conjugation (left) versus biotin-streptavidin conjugation (right).

FIGS. 7A-7C: Phagocytosis of functionalized nanoparticles by macrophages. FIG. 7A is a series of epifluorescent images of varying sizes of nanoparticles (left, 200 nanometers (nm) average diameter; middle 500 nm average diameter; right 1200 nm average diameter) labelled with a fluorescent dye. FIG. 7B is a graph showing the measured zeta potentials (measured in millivolts (mV)) of functionalized nanoparticles of the presently disclosed subject matter with varying phosphatidylserine (PS) lipid ratios (0%, 5%, 10%, or 15%) in the lipid layer. FIG. 7C is a graph of interleukin-6 (IL-6) measured (in nanograms per milliliter (ng/mL)) via enzyme-linked immunosorbent assay (ELISA) conducted on supernatant from macrophages with or without lung epithelial cells, severe acute respiratory syndrome coronavirus 2 (SARS-CoV-2) pseudovirus, or exemplary nanoparticles of the presently disclosed subject matter (i.e., angiotensin-converting enzyme 2 (ACE2)-functionalized nanoparticles (Nanotrap-ACE2)). MΦ: macrophages; Virus: SARS-CoV-2 pseudovirus; Epithelial: epithelial cells; Nanotrap: Nanotrap-ACE2. No significant statistical differences were found between any groups.

FIGS. 8A-8E: Inhibition of severe acute respiratory syndrome coronavirus 2 (SARS-CoV-2) pseudovirus in HEK293T-ACE2 cells. FIGS. 8A and 8B are graphs of infectivity (measured as percent (%) infectivity) in HEK293T-ACE2 cells treated with SARS-CoV-2 spike pseudotyped lentivirus (FIG. 8A) or vesicular stomatitis virus (VSV; FIG. 8B) and varying concentrations (indicated on the x-axis in micrograms per milliliter (μg/mL)) of angiotensin-converting enzyme 2 (ACE2)-functionalized nanoparticles (Nanotrap-ACE2), neutralizing antibody-functionalized nanoparticles (Nanotrap-Antibody), or nanoparticles with no virus targeting functionalization (Nanotrap-Blank) for 72 and 24 hours, respectively. Data are presented as mean±standard deviation (SD), unpaired t tests were conducted on three independent replicates. FIGS. 8C and 8D are graphs infectivity (measured as percent (%) infectivity) of HEK293T-ACE2 cells treated with SARS-CoV-2 spike pseudotyped lentivirus (FIG. 8C) or VSV (FIG. 8D) and varying amounts of Nanotrap-ACE2 or soluble ACE2 for 72 and 24 hours, respectively. Amount of ACE2 is indicated on the x-axis in μg/mL. Data are presented as mean±SD; unpaired t tests were conducted on three independent replicates. FIG. 8E is a series of representative fluorescent images of VSV-green fluorescent protein (GFP) pseudovirus infected HEK293T-ACE2 cells after treatment with different concentrations (as indicated at the top in μg/mL) of ACE2 protein (top series of images) or Nanotraps-ACE2 (bottom series of images). *p<0.05, **p<0.005, ***p<0.0005, ****p<0.00005. N=3 per group.

FIGS. 9A-9B: In vitro biosafety profile of functionalized nanoparticle treatment. FIGS. 9A and 9B show the cell viability (measured as a percentage) of HEK293T-ACE2 (FIG. 9A) and A549 (FIG. 9B) cells treated with different treatments: a nanoparticle with no anti-viral functionalization (Nanotrap-Blank) an angiotensin-converting enzyme 2 (ACE2)-functionalized nanoparticle (Nanotrap-ACE2), an anti-viral antibody-functionalized antibody (Nanotrap-Antibody) or no treatment (untreated) for 72 hours. Data are presented as mean±standard deviation SD; n=5 per group.

FIGS. 10A-10E: Ex vivo human lung perfusion (EVLP) system for evaluating the neutralizing ability of functionalized nanoparticles of the presently disclosed subject matter. FIG. 10A is a graph of severe acute respiratory syndrome coronavirus 2 (SARS-CoV-2) pseudotyped lentivirus infectivity (measured as percent (%) infectivity) of primary human cells in vitro 8 or 48 hours post infection as a function of virus volume (0, 10, or 25 microliters (μL)). FIG. 10B is a graph of SARS-CoV-2 pseudotyped lentivirus infectivity (measured as % infectivity) of primary human cells in vitro 48 hours post infection as a function of virus volume (0, 5, 10, 15, 20, or 25 μL). FIG. 10C is a graph of the quantification of lung compliance and oxygenation capacity while on EVLP. FIG. 10D is a graph showing the quantification of fluorescence of ex vivo lung tissue sections stained with fluorescent dye (DAPI). Auto-fluorescence from the 560 nanometer (nm) wavelength channel was quantified and normalized to signal from 488 nm wavelength. FIG. 10E is a graph of infectivity of Vero E6 cells treated with authentic SARS-CoV-2 and anti-viral antibody-functionalized nanoparticle (Nanotrap-Antibody) or soluble neutralizing antibody. Antibody concentrations on the functionalized nanoparticle were calculated by multiplying the particle concentration per mL with antibody density per particle measured in FIGS. 6F and 6G. Data are shown as mean±SD fitted with a trend curve.

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. Definitions

While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “a metal ion” includes a plurality of such metal ions, and so forth.

Unless otherwise indicated, all numbers expressing quantities of size, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value or to an amount of size (i.e., diameter), weight, concentration or percentage is meant to encompass variations of in one example ±20% or ±10%, in another example ±5%, in another example ±1%, and in still another example ±0.1% from the specified amount, as such variations are appropriate to perform the disclosed methods.

Numerical ranges recited herein by endpoints include all numbers and fractions subsumed within that range (e.g. 1 to 5 includes, but is not limited to, 1, 1.5, 2, 2.75, 3, 3.90, 4, and 5).

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including,” “containing,” or “characterized by” is inclusive or open-ended and does not exclude additional, unrecited elements or method steps. “Comprising” is a term of art used in claim language which means that the named elements are present, but other elements can be added and still form a construct or method within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

The terms “bonding” or “bonded” and variations thereof can refer to either covalent or non-covalent bonding. In some cases, the term “bonding” refers to bonding via a coordinate bond. The term “conjugation” can refer to a bonding process, as well, such as the formation of a covalent linkage or a non-covalent bond.

As used herein, the term “ligand” refers generally to a species, such as a molecule or ion, which interacts, e.g., binds, in some way with another species.

“Biodegradable” means materials that are broken down or decomposed by natural biological processes. Biodegradable materials can be broken down for example, by cellular machinery, proteins, enzymes, hydrolyzing chemicals or reducing agents present in biological fluids or soil, intracellular constituents, and the like, into components that can be either reused or disposed of without significant toxic effect on the environment. Thus, the term “biodegradable” as used herein refers to both enzymatic and non-enzymatic breakdown or degradation of polymeric structures into lower weight materials (e.g., oligomers or non-polymeric compounds). In some embodiments, the degradation time is a function of polymer composition and morphology. Suitable degradation times are from hours or days to weeks to years.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in an animal. In some embodiments, a pharmaceutically acceptable carrier is pharmaceutically acceptable for use in a human.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the presently disclosed subject matter can be of any of the five major classes or corresponding sub-classes. Typically, the antibodies are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the presently disclosed subject matter can contain a kappa or lambda light chain constant domain. According to some embodiments, the antibodies of the presently disclosed subject matter include heavy and/or light chain constant regions from rat, mice, rabbit, or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

The term an “isolated antibody” refers to an antibody which is substantially free of other antibodies having different antigenic specificities (e.g., an isolated antibody that specifically binds to a spike protein of SARS-CoV-2 is substantially free of antibodies that do not bind to the spike protein). In addition, an isolated antibody is substantially free of other cellular material and/or chemicals.

The term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies of can be made by the hybridoma method, phage display technology, single lymphocyte gene cloning technology, or by recombinant DNA methods. For example, monoclonal antibodies can be produced by a hybridoma which includes a B cell obtained from a transgenic nonhuman animal, such as a transgenic mouse or rat, having a genome comprising a human heavy chain transgene and a light chain transgene.

The term “antigen-binding fragment thereof” refers to a fragment of an antibody, such as, for example, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdab) an scFv dimer (bivalent diabody), a multispecific antibody formed from a portion of an antibody (e.g., a virus-neutralizing antibody) comprising one or more CDRs, a camelized single domain antibody, a nanobody, a domain antibody, a bivalent domain antibody, or any other antibody fragment that binds to an virus-related antigen but does not comprise a complete antibody structure. Thus, a binding fragment of an antibody is capable of binding to the same antigen to which the parent antibody or a parent antibody fragment binds. In some embodiments, the binding fragment comprises a light chain variable region, a light chain constant region, and an Fd segment of the heavy chain. According to some embodiments, the binding fragment comprises Fab and F(ab′).

As used herein, the term “single-chain antibody” refers to a conventional single-chain antibody in the field, which comprises a heavy chain variable region and a light chain variable region connected by a short peptide of about 15 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes overlap or substantially overlap. In some embodiments, the first and second epitopes do not overlap or do not substantially overlap. In some embodiments, the first and second epitopes are on different antigens, e.g., different proteins (or different subunits of a multimeric protein). In some embodiments, a multispecific antibody comprises a third, fourth, or fifth immunoglobulin variable domain. In some embodiments, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). In some embodiments, the first and second epitopes overlap or substantially overlap. In some embodiments, the first and second epitopes are on different antigens, e.g., different proteins (or different subunits of a multimeric protein). In some embodiments, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope.

The term “pathogen-binding antibody” refers to an antibody that specifically binds to a particular pathogen or class of pathogens. In some embodiments, the term “pathogen-binding antibody” refers to an antibody that binds to a pathogen, e.g., a bacterial surface protein or a viral protein, such as, but not limited to a spike protein of a coronavirus such as SARS-CoV-2, with a KD of 1×10⁻⁷ M or less, 1×10⁻⁸ M or less, 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a BIACORE™ system, or by using bio-layer interferometry technology, such as an Octet RED96 system. The smaller the value of the KD of an antibody (or antigen-binding fragment thereof), the higher affinity that the antibody (or fragment) binds to a target antigen.

Some pathogen-binding antibodies (e.g., some virus-binding antibodies) can also be referred to “neutralizing” antibodies. As used herein, a “neutralizing antibody” or pathogen-neutralizing antibody” refers to an antibody that defends a host or host cell from an infectious agent by neutralizing any effect (e.g., cytotoxicity, infectivity, etc.) it has biologically. For example, a “neutralizing antibody” can bind specifically to surface structures (antigens) on an infectious particle (e.g., a virus), to prevent the particle from interacting with host cells it might infect and destroy. A “virus-neutralizing antibody, for instance, can be an antibody that not only binds to a virus, but also binds in a manner that blocks viral infection. Thus, a “viral-neutralizing antibody” can be an antibody that blocks interactions of the virus with a receptor or can bind to a viral capsid such that the uncoating of the viral genome is inhibited. A “non-neutralizing antibody” refers to an antibody that specifically binds to an infectious agent but is incapable of ameliorating the biological effects of the infectious agent on the host cell. A “sub-neutralizing antibody” refers to an antibody that is capable of partially neutralizing the biological effects of the infectious agent on the host cell. A sub-neutralizing antibody can ameliorate one or more biological effects of an infectious agent on the host cell by no more than about any one of 90%, 80%, 70%, 60%, 50%, 40%, 30%, 20%, 10% or less compared to a neutralizing antibody.

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage. The term can also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response can involve either antibody production, or the activation of specific immunologically-competent cells, or both. An antigen can be derived from organisms, subunits of proteins/antigens, killed or inactivated whole cells or lysates. The term “immunogen” is used interchangeably with “antigen” herein.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The terms “nanoscale particle,” “nanomaterial,” and “nanoparticle” refer to a structure having at least one region with a dimension (e.g., length, width, diameter, etc.) of less than about 5000 nm, less than about 2500 nm, less than about 2000 nm, less than about 1500 nm, less than about 1250 nm, or less than about 1000 nm. In some embodiments, the dimension is smaller (e.g., less than about 500 nm, less than about 250 nm, less than about 200 nm, less than about 150 nm, less than about 125 nm, less than about 100 nm, less than about 80 nm, less than about 70 nm, less than about 60 nm, less than about 50 nm, less than about 40 nm, less than about 30 nm or even less than about 20 nm). In some embodiments, the dimension is between about 20 nm and about 250 nm (e.g., about 20, 30, 40, 50, 60, 70, 80, 90, 100, 110, 120, 130, 140, 150, 160, 170, 180, 190, 200, 210, 220, 230, 240, or 250 nm).

In some embodiments, the nanoparticle is approximately spherical. When the nanoparticle is approximately spherical, the characteristic dimension can correspond to the diameter of the sphere. In addition to spherical shapes, the nanomaterial can be disc-shaped, plate-shaped (e.g., hexagonally plate-like), oblong, polyhedral, rod-shaped, cubic, or irregularly-shaped.

The nanoparticle can comprise a core region (i.e., the space between the outer dimensions of the particle) and an outer surface (i.e., the surface that defines the outer dimensions of the particle). In some embodiments, the nanoparticle can have one or more coating layers surrounding or partially surrounding the nanoparticle core. Thus, for example, a spherical nanoparticle can have one or more concentric coating layers, each successive layer being dispersed over the outer surface of a smaller layer closer to the center of the particle. Such nanoparticles can be referred to as “core-shell” nanoparticles, wherein the shell refers to the coating layer or layers.

The term “small molecule” as used herein can refer to a non-polymeric, naturally-occurring or synthetic molecule. Small molecules typically have a molecular weight of about 900 Daltons (Da) or less (e.g., about 800 Da, about 750 Da, about 700 Da, about 650 Da, about 600 Da, about 550 Da, or about 500 Da or less).

The term “macromolecule” as used herein refers to molecules that are larger than about 900 Da. In some embodiments, the macromolecule is a polymer or biopolymer, e.g., a protein or a nucleic acid.

The terms “polymer” and “polymeric” refer to chemical structures that have repeating units (i.e., multiple copies of a given chemical substructure). Polymers can be formed from polymerizable monomers. A polymerizable monomer is a molecule that comprises one or more moieties that can react to form bonds (e.g., covalent or coordination bonds) with moieties on other molecules of polymerizable monomer. In some embodiments, each polymerizable monomer molecule can bond to two or more other molecules/moieties. In some cases, a polymerizable monomer will bond to only one other molecule, forming a terminus of the polymeric material.

Polymers can be organic, or inorganic, or a combination thereof. As used herein, the term “inorganic” refers to a compound or composition that contains at least some atoms other than carbon, hydrogen, nitrogen, oxygen, sulfur, phosphorous, or one of the halides. Thus, for example, an inorganic compound or composition can contain one or more silicon atoms and/or one or more metal atoms.

As used herein “organic polymers” are those that do not include silica or metal atoms in their repeating units. Exemplary organic polymers include polyvinylpyrrolidone (PVO), polyesters, polyamides, polyethers, polydienes, and the like. Some organic polymers contain biodegradable linkages, such as esters or amides, such that they can degrade overtime under biological conditions.

The term “hydrophilic polymer” as used herein generally refers to hydrophilic organic polymers, such as but not limited to, polyvinylpyrrolidone (PVP), polyvinylmethylether, polymethyloxazoline, polyethyloxazoline, polyhydroxy-propyloxazoline, polyhydroxypropylmethacrylamide, polymethyacrylamide, polydimethylacrylamide, polyhydroxylpropylmethacrylate, polyhydroxy-ethylacrylate, hydroxymethylcellulose, hydroxyethylcellulose, polyethylene-imine (PEI), polyethyleneglycol (i.e., PEG) or another hydrophilic poly(alkyleneoxide), polyglycerine, and polyaspartamide. As noted above, the term “hydrophilic” refers to the ability of a molecule or chemical species to interact with water. Thus, hydrophilic polymers are typically polar or have groups that can hydrogen bond to water.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the presently disclosed subject matter refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

The term “prevent”, as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

The term “coronavirus” as used herein refers to a member of a family of positive-sense, single-stranded RNA viruses of the subfamily Orthocoronavirinae, in the family Coronaviridae, order Nidovirales, and realm Riboviria. The viral genome is capped, polyadenylated, and covered with nucleocapsid (N) proteins. The coronavirus virion includes a viral envelope containing type I fusion glycoproteins referred to as the spike (S) protein. Most coronaviruses have a common genome organization with the replicase gene included in the 5′-portion of the genome, and structural genes included in the 3′-portion of the genome. Coronaviruses have four genera: alpha-, beta-, gamma-, and delta-coronaviruses. The genome size of coronaviruses range from approximately 26 to 32 kilobases. Non-limiting examples of betacoronaviruses include Middle East respiratory syndrome coronavirus (MERS-CoV), Severe Acute Respiratory Syndrome coronavirus (SARS-CoV), Severe Acute Respiratory Syndrome coronavirus 2 (SARS-CoV-2 or COVID-19), Human coronavirus HKU1 (HKU1-CoV), Human coronavirus OC43 (OC43-CoV), Murine Hepatitis Virus (MHV-CoV), Bat SARS-like coronavirus WIV1 (WIV1-CoV), and Human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV). A non-limiting example of a deltacoronaviruses is the Swine Delta Coronavirus (SDCV)

As used herein, the term “severe acute respiratory syndrome coronavirus 2 (SARS-CoV-2)” refers to a virus comprising a virion 50-200 nanometers in diameter and a genomic size of about 30 kilobases, encoding multiple structural proteins, such as the S (spike), E (envelope), M (membrane) and N (nucleocapsid), and non-structural proteins. As used herein, the term “S protein” or “Spike protein” is used to refer to a knoblike structured (i.e., spikes) peplomer, which is composed of glycoprotein to project from the lipid bilayer of the surface envelope of an enveloped virus.

Coronaviruses can cause diseases in mammals and birds. In humans and birds, they cause respiratory tract infections that can range from mild to lethal. Mild illnesses in humans include some cases of the common cold (which is also caused by other viruses, predominantly rhinoviruses), while more lethal varieties can cause SARS, MERS, and COVID-19. Since SARS-CoV-2 shares 80% sequence homology with SARS-CoV-1, antibodies against S protein in SARS-CoV-2 can also bind SARS-Cov-1. Thus, some of the nanoparticles of the presently disclosed subject matter useful in treating or preventing SARS-CoV-2 can also be used to treat or prevent infections of other virus types such as SARS-CoV (i.e., SARS-CoV-1) and MERS-CoV that are similar in virion structure.

### II. Representative Methods and Compositions

To gain entry to host cells for infection, SARS-CoV-2 surface spike protein binds to its receptor human angiotensin-converting enzyme 2 (ACE2) with high affinity⁴⁻⁷. Blocking entry of SARS-CoV-2 to host cells can prevent infection. To achieve this goal, both soluble recombinant ACE2 proteins^{8,9} and anti-SARS-CoV-2 neutralizing antibodies¹⁰⁻¹⁴ have been developed to inhibit the interaction between SARS-CoV-2 spike protein and cell-surface ACE2, although they have shown limited potency^{8,9,15}.

In some embodiments, the presently disclosed subject matter provides a therapeutic functionalized synthetic nanoparticle, referred to herein as a “Nanotrap” to inhibit SARS-CoV-2 infection or another pathogen infection. According to one aspect of the design of the nanoparticles, the nanoparticle surface is functionalized with a high surface density of pathogen receptors (e.g., ACE2 recombinant proteins) or anti-pathogen (e.g., anti-SARS-CoV-2) antibodies. The functionalized nanoparticles can thus mimic the target cells to ensnare a pathogen, such a virus or bacteria. Meanwhile, the polymer-lipid complexes of the nanoparticle structure take advantage of both the stability from polymers and the surface flexibility of lipids⁴⁸, thereby providing a well-controlled nanomaterial with a high capacity to trap pathogens.

Exemplary functionalized nanoparticles described herein comprise a FDA-approved polylactic acid (PLA) polymeric core, a liposome shell, surface ACE2/neutralizing antibodies, and phosphatidylserine ligands. As described below, the nanoparticles blocked pseudotyped SARS-CoV-2 entry into susceptible ACE2-overexpressing HEK293T cells, lung epithelial A549 cells, and human primary lung cells, as well as authentic SARS-CoV-2 infection of Vero E6 cells. Subsequently, macrophages efficiently engulfed and neutralized functionalized nanoparticles-virus complexes through phosphatidylserine-guided phagocytosis without causing infection to macrophages in vitro. Furthermore, in vitro cell culture and in vivo intratracheal administration of exemplary functionalized nanoparticles to immunocompetent mice demonstrated an excellent biosafety profile. Additionally, exemplary functionalized nanoparticles of the presently disclosed subject matter inhibited infection of SARS-CoV-2 pseudovirus in live human lungs maintained under normothermic physiologic conditions on a clinically applicable ex vivo lung perfusion (EVLP) system^{24,25}, further confirming therapeutic efficacy. Accordingly, the presently disclosed nanoparticles are safe, effective, biocompatible, ready for mass production, and convenient to use. They provide a new type of nanomedicine to effectively contain and clear SARS-CoV-2 for the prevention and treatment of COVID-19 or other diseases caused by pathogens.

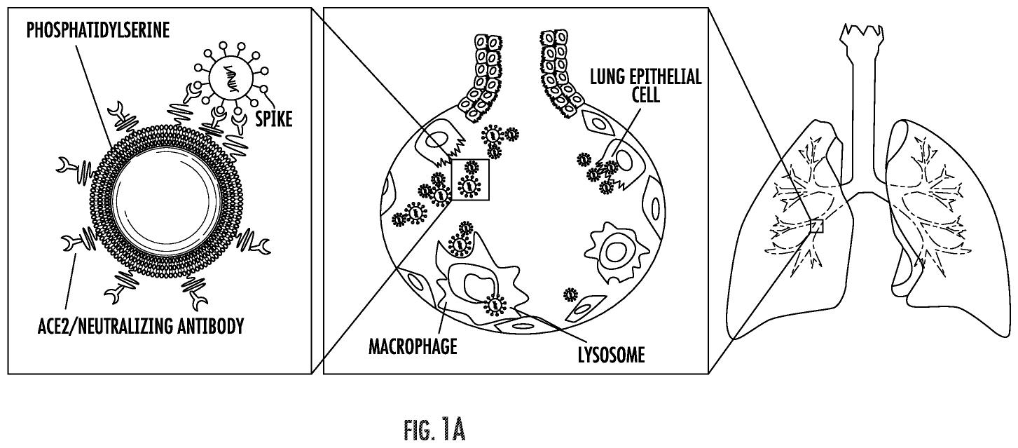

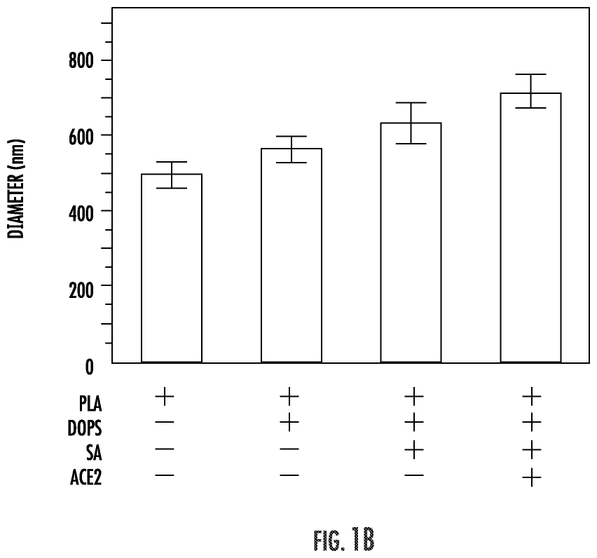

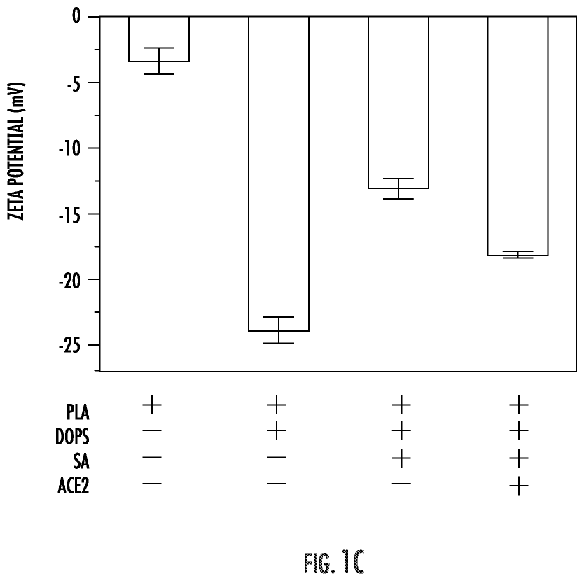

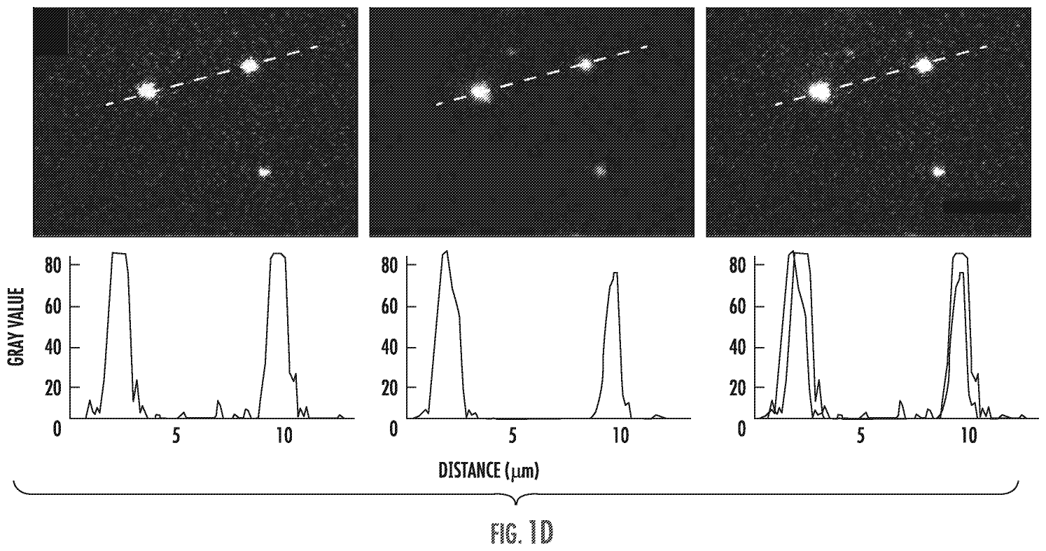

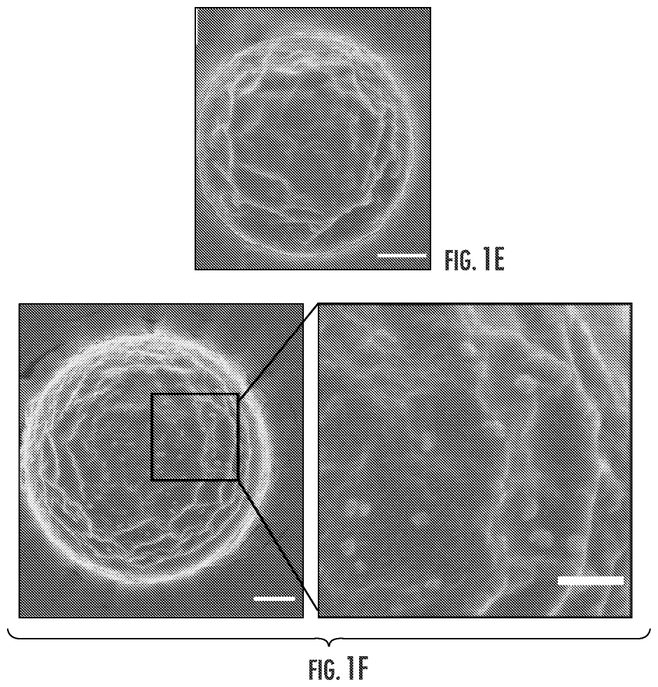

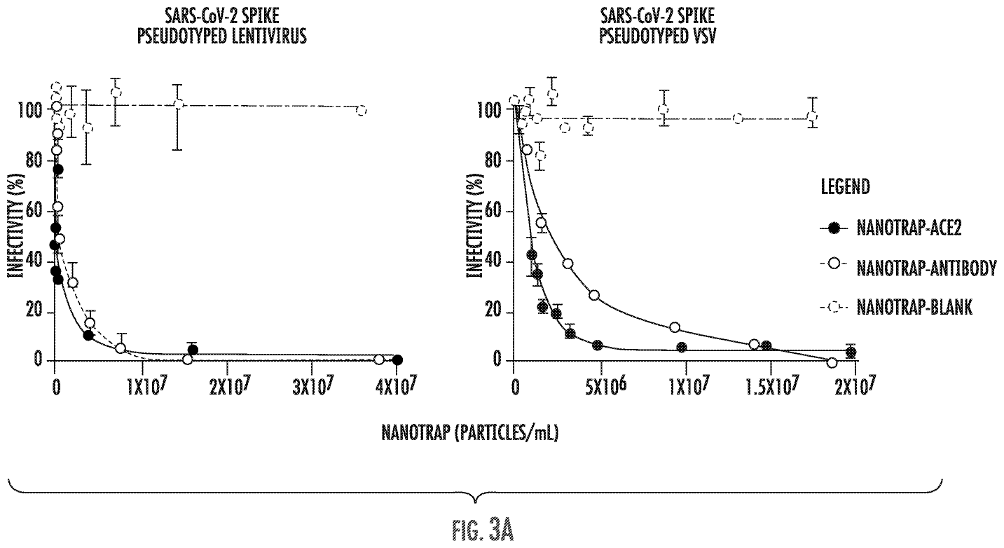

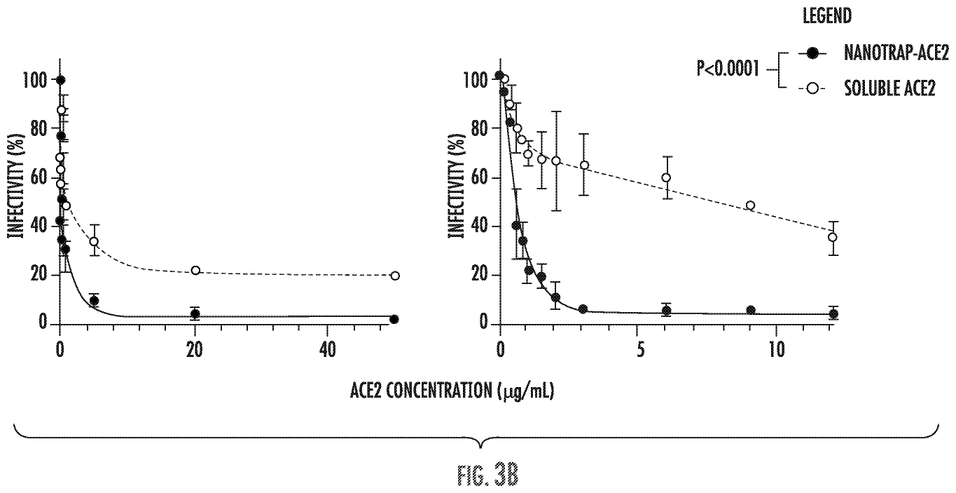

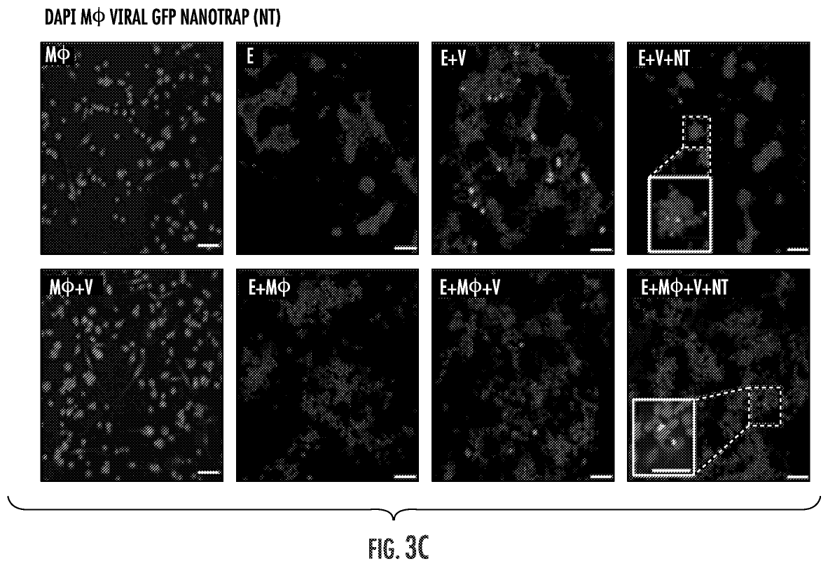

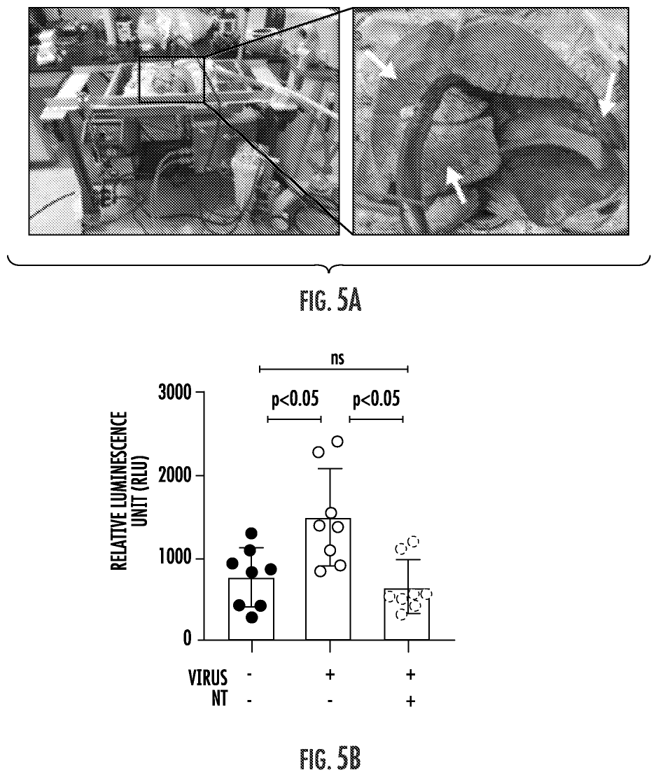

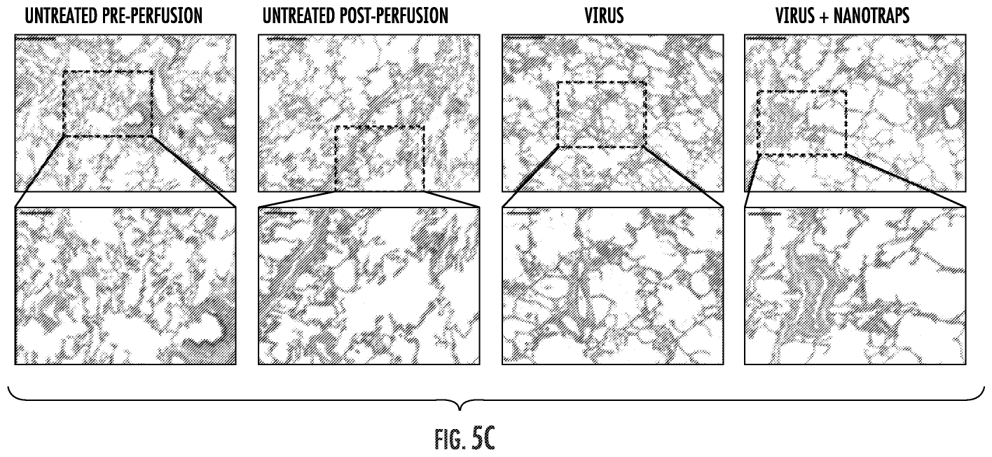

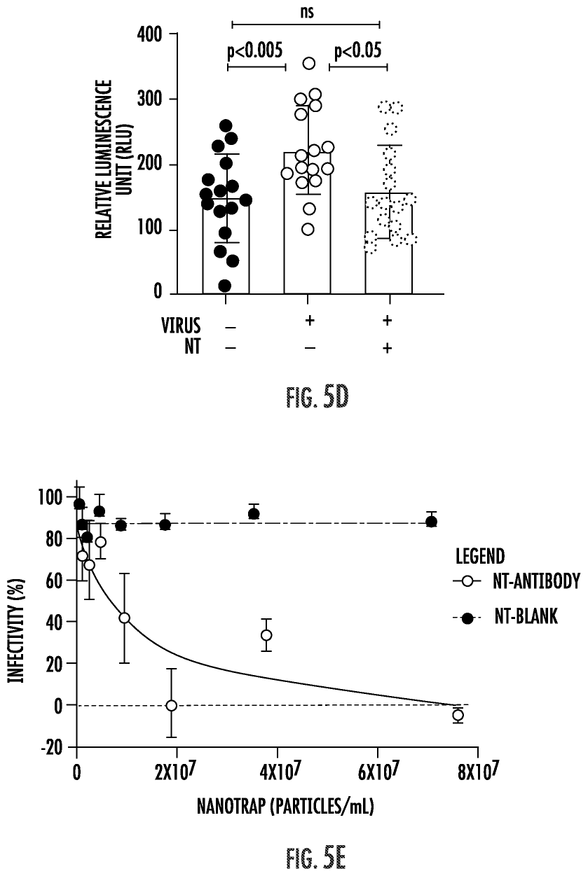

More particularly, to block the interaction between the SARS-CoV-2 spike protein and the host ACE2 receptors, exemplary nanoparticle surfaces were functionalized with a high molecular density of either recombinant ACE2 proteins or anti-SARS-CoV-2 neutralizing antibodies. See FIG. 1A. The high binding avidity, high diffusivity, and small size of the functionalized nanoparticles can provide them the ability to easily outcompete low ACE2-expressing host cells in capturing SARS-CoV-2, thus effectively containing the virus on their surfaces. The studies described herein below demonstrated that viral infection of both pseudotyped and authentic SARS-CoV-2 across human cell lines, lung primary cells and lung organs can be inhibited (e.g., completely inhibited) by the exemplary functionalized nanoparticles of the presently disclosed subject matter, i.e., ACE2-functionalised nanoparticles (referred to herein as Nanotrap-ACE2) or anti-virus antibody-functionalized nanoparticles (referred to herein as Nanotrap-Antibody). See FIGS. 3A-3D and 5A-5E. Notably, Nanotrap-ACE2 was superior to soluble recombinant ACE2 proteins in containing SARS-CoV-2^{8,9}, which, without being bound to any one theory is attributed to the high binding avidity of the functionalized nanoparticles. See FIG. 3B.

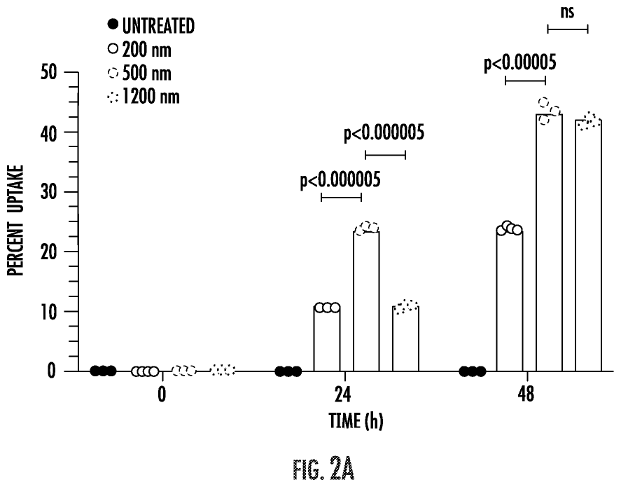

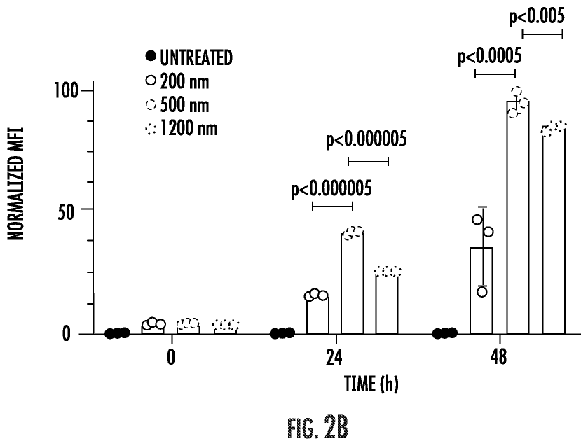

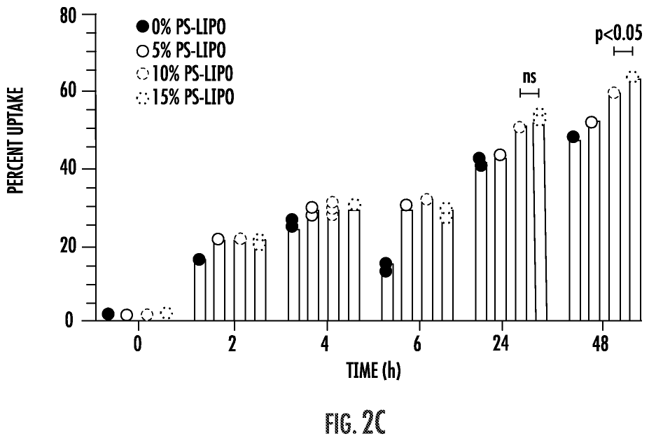

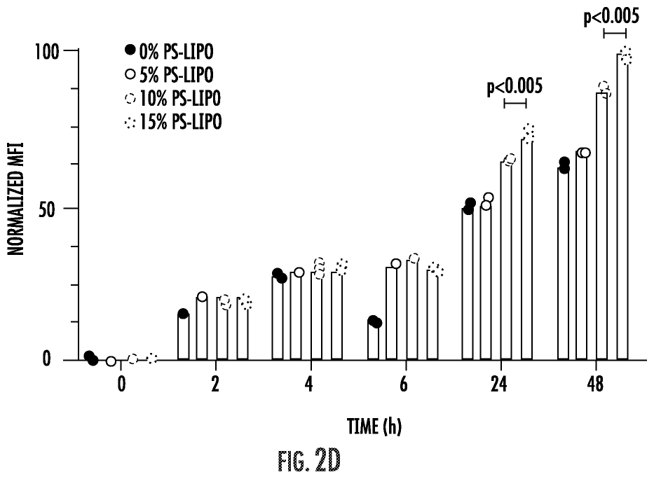

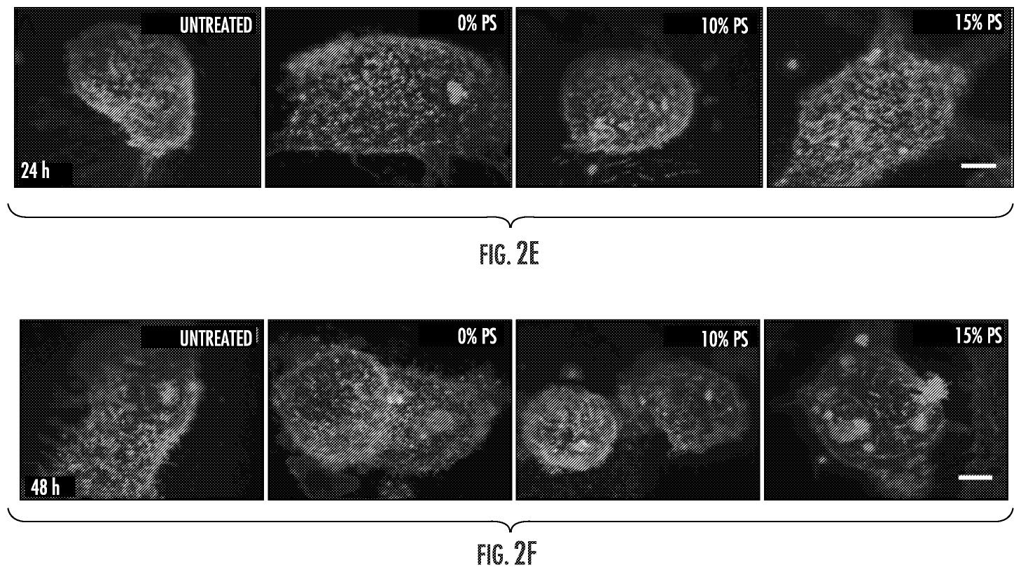

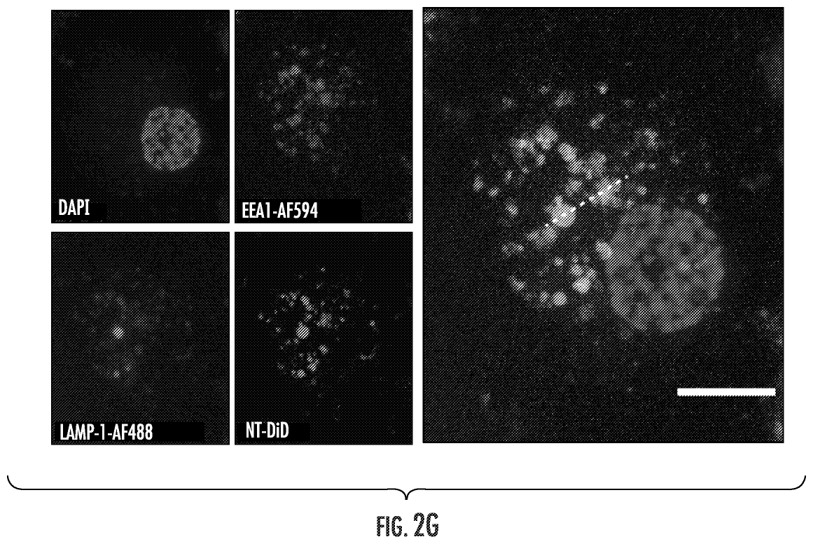

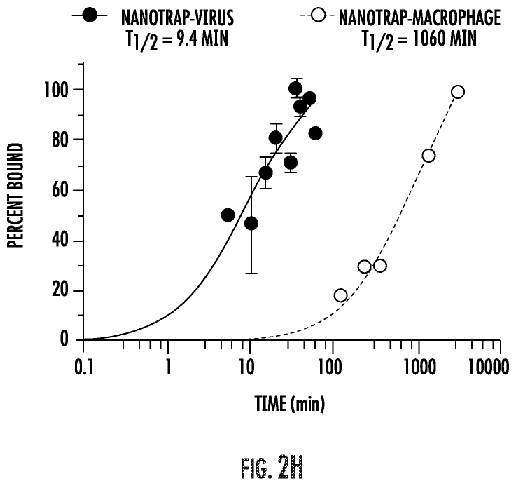

Furthermore, the presently disclosed functionalized nanoparticles harness the immune system to clear the SARS-CoV-2. See FIGS. 2A-2F and 3A-3D. By incorporating phagocyte-specific phosphatidylserine ligands onto the nanoparticle surfaces, macrophages readily engulfed the virus-bound nanoparticles without becoming infected themselves. See FIGS. 3C and 3D. The role of macrophages in the control of infections has been documented¹⁷, and recent single-cell RNA sequencing found abundant monocyte-derived macrophages in the bronchoalveolar lavage fluid of COVID-19 patients¹⁸. While macrophages were used as a proof-of-principle in this study, it is expected that other professional phagocytes such as neutrophils, monocytes, and dendritic cells can similarly clear the virus-bound nanoparticles of the presently disclosed subject matter. In particular, macrophages and dendritic cells are professional antigen presenting cells, which present engulfed antigens to the adaptive immune system⁴⁹. Since the presently disclosed functionalized nanoparticles are able to engage antigen presenting cells, it is believed possible that they can also elicit virus-specific adaptive immune responses, thereby promoting vaccine-like protection⁵⁰.

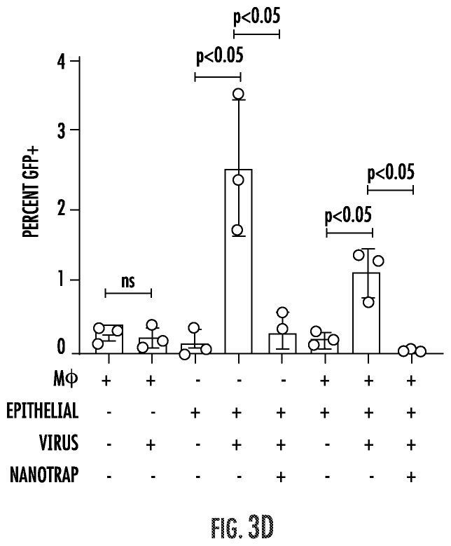

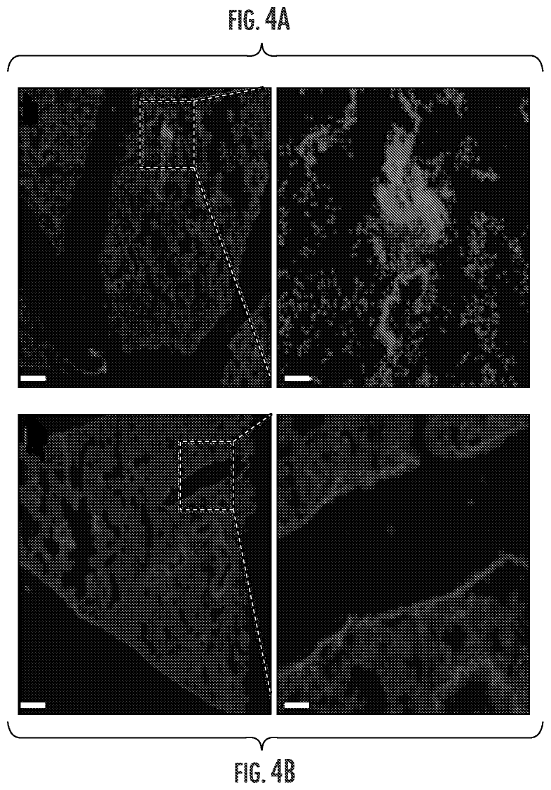

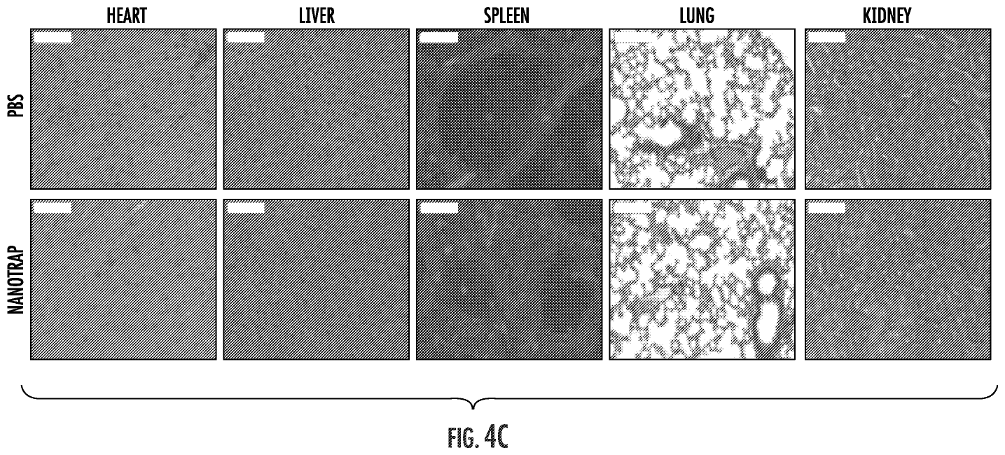

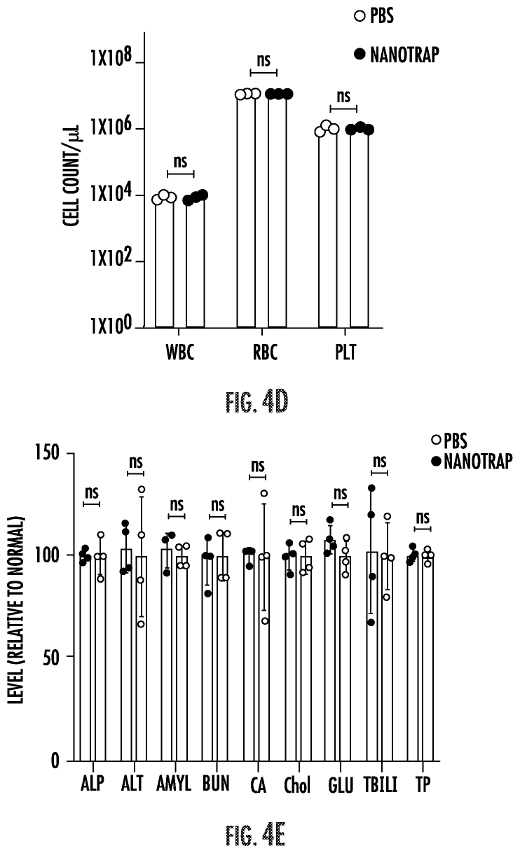

In addition, the presently disclosed functionalized nanoparticles were designed to be biocompatible, biodegradable, and safe. The functionalized nanoparticles are composed of FDA-approved polymers and lipids, which provides for safe administration in a clinical setting. Biosafety experiments demonstrated an excellent safety profile in vitro and in vivo. See FIGS. 4A-4E.

The efficacy of the presently disclosed functionalized nanoparticles was studied in a human EVLP system. Superior to lung organoids which cannot reproduce whole-organ response to viral infection^{9,51,52}, and non-human primate models which are extremely costly⁵³⁻⁵⁵, the EVLP system is a clinically relevant model. The presently disclosed functionalized nanoparticles can inhibit (e.g., completely inhibit) viral infection in living human lungs. See FIGS. 5A-5E. As current biosafety regulations preclude the testing of authentic SARS-CoV-2 in the EVLP, it was confirmed that the presently disclosed nanoparticles can inhibit authentic virus in vitro. See FIG. 5E. These experiments together suggest that the presently disclosed functionalized nanoparticles can be used to treat SARS-CoV-2 infection in the clinic.

Accordingly, in some embodiments, described herein is a new type of potent, effective nanomedicine, i.e., functionalized nanoparticles or “Nanotraps,” to contain and clear pathogens, such as viruses (e.g., SARS-CoV-2) and bacteria, by harnessing and integrating the power of nanotechnology and immunology. As an example, the presently disclosed nanoparticles inhibited SARS-CoV-2 infection of human cells and lung organs. The functionalized nanoparticles are effective, biocompatible, safe, stable, feasible for mass production. The functionalized nanoparticles can be formulated into a nasal spray or inhaler for easy administration and direct delivery to the respiratory system, or as an oral or ocular liquid, or for subcutaneous, intramuscular or intravenous injection to target different sites of pathogen (e.g., SARS-CoV-2) exposure, thus offering flexibility in administration. Furthermore, the design of the presently disclosed functionalized nanoparticles is highly versatile: they can be modified to incorporate small molecule drugs or protein/mRNA vaccines into their core, while different pathogen-binding receptors and/or pathogen-binding antibodies (or the antigen-binding fragments thereof), such as, but not limited to different human ACE2 recombinant proteins”^{8,9}, human anti-SARS-CoV-2 neutralizing antibodies¹⁰⁻¹⁴, as well as other therapeutic proteins or peptides can be conjugated to the surface.

In some embodiments, the presently disclosed subject matter provides a nanoparticle comprising: (a) a core comprising a biocompatible polymer; and (b) an outer layer encapsulating said core, wherein the outer layer comprises one or more lipids and/or one or more proteins, and wherein the outer surface of the outer layer comprises: (c) a pathogen-binding receptor and/or a pathogen-binding antibody or an antigen-binding fragment thereof; and (d) a phagocyte-specific ligand.

In some embodiments, the biocompatible polymer comprises a biodegradable polymer. The biodegradable polymer can be a homopolymer or a copolymer, including random copolymer, block copolymer, or graft copolymer. The biodegradable polymer can be a linear polymer, a branched polymer, or a dendrimer. The biodegradable polymers can be of natural or synthetic origin. Examples of suitable biodegradable polymers include, but are not limited to polymers such as those made from lactide, glycolide, caprolactone, valerolactone, carbonates (e.g., trimethylene carbonate, tetramethylene carbonate, and the like), dioxanones (e.g., 1,4-dioxanone), 6-valerolactone, 1,dioxepanones) e.g., 1,4-dioxepan-2-one and 1,5-dioxepan-2-one), ethylene glycol, ethylene oxide, esteramides, γ-hydroxyvalerate, β-hydroxypropionate, α-hydroxy acid, hydroxybuterates, poly (ortho esters), hydroxy alkanoates, tyrosine carbonates, polyimide carbonates, polyimino carbonates such as poly (bisphenol A-iminocarbonate) and poly (hydroquinone-iminocarbonate, (polyurethanes, polyanhydrides, polymer drugs (e.g., polydiflunisol, polyaspirin, and protein therapeutics) and copolymers and combinations thereof. Suitable natural biodegradable polymers include those made from collagen, chitin, chitosan, cellulose, poly (amino acids), polysaccharides, hyaluronic acid, gut, copolymers and derivatives and combinations thereof. In some embodiments, the biodegradable polymer is a copolymer or terpolymer, for example: polylactides (PLA), poly-L-lactide (PLLA), poly-DL-lactide (PDLLA); polyglycolide (PGA); copolymers of glycolide, glycolide/trimethylene carbonate copolymers (PGA/TMC); other copolymers of PLA, such as lactide/tetramethylglycolide copolymers, lactide/trimethylene carbonate copolymers, lactide/6-valerolactone copolymers, lactide/ε-caprolactone copolymers, L-lactide/DL-lactide copolymers, glycolide/L-lactide copolymers (PGA/PLLA), polylactide-co-glycolide; terpolymers of PLA, such as lactide/glycolide/trimethylene carbonate terpolymers, lactide/glycolide/ε-caprolactone terpolymers, PLA/polyethylene oxide copolymers; polydepsipeptides; unsymmetrically 3,6-substituted poly-1,4-dioxane-2,5-diones; polyhydroxyalkanoates; such as polyhydroxybutyrates (PHB); PHB/β-hydroxyvalerate copolymers (PHB/PHV); poly-β-hydroxypropionate (PHPA); poly-p-dioxanone (PDS); poly-δ-valerolactone-poly-ε-capralactone, poly(ε-caprolactone-DL-lactide) copolymers; methylmethacrylate-N-vinyl pyrrolidone copolymers; polyesteramides; polyesters of oxalic acid; polydihydropyrans; polyalkyl-2-cyanoacrylates; polyurethanes (PU); polyvinylalcohol (PVA); polypeptides; poly-β-malic acid (PMLA); polycarbonates; polyorthoesters; polyphosphates; poly(ester anhydrides); and mixtures thereof; and natural polymers, such as sugars; starch, cellulose and cellulose derivatives, polysaccharides, collagen, chitosan, fibrin, hyaluronic acid, polypeptides and proteins. Mixtures of any of the above-mentioned polymers and their various forms can also be used. In some embodiments, the biodegradable polymer comprises a polyester, a polyether, or a polyamide. In some embodiments, the biodegradable polymer comprises polylactic acid (PLA), poly(lactic-co-glycolic acid) (PLGA), a PLGA-PLA copolymer, polypyrrole, or a mixture thereof.

In some embodiments, the core further comprises perfluorooctyl bromide (PFOB). In some embodiments, the nanoparticle core can include up to about 65% by weight PFOB. In some embodiments, the core comprises about 10% to about 65% (e.g., about 10%, 15%, 20%, 25%, 30%, 35%, 40%, 45%, 50%, 55%, 60%, or about 65%) by weight PFOB. In some embodiments, the nanoparticle core comprises about 20% to about 25% PFOB. In some embodiments, the addition of PFOB (or larger amounts of PFOB) can provide larger nanoparticle cores. In addition, inclusion of PFOB in the core can help to stabilize the whole nanoparticle. Further, as PFOB has a density of 793 g/cm³, nearly 2 times heavier than water, incorporating PFOB in the nanoparticle core can result in a nanoparticle that can be more easily concentrated. For example, in some embodiments, the presently disclosed nanoparticles can be concentrated at a relative low speed (e.g., about 3,000 g) over a relatively short period of time (e.g., about 5 minutes). Concentration of other nanoparticles known in the art, (i.e., not of the presently disclosed subject matter), such as liposomes, can typically involve ultracentrifugation (e.g., at 30,000 g) and/or longer concentration times (e.g., 30 minutes). In addition, PFOB has the capability to carry oxygen. Thus, inclusion of PFOB in the nanoparticles could be utilized to relieve hypoxia. PFOB could also be included in the core as a contrast agent, e.g., to enhance or provide for computed tomography (CT) and/or ultrasound imaging of the nanoparticles. Thus, in some embodiments, the presently disclosed nanoparticles can be used to diagnose infection and/or the presence of a pathogen.

In some embodiments, the nanoparticle core has a diameter (i.e., an average diameter, such as an average diameter as measured by dynamic light scattering (DLS)) between about 200 and about 1200 nm (e.g., about 200, 300, 400, 500, 600, 700, 800, 900, 1000, 1100, or 1200 nm). In some embodiments, the core has a diameter between about 300 nm and about 700 nm (e.g., about 300, 325, 350, 375, 400, 425, 450, 475, 500, 525, 550, 575, 600, 625, 650, 675 or about 700 nm. In some embodiments, the core as a diameter of about 500 nm.

Any suitable receptor known in the art that can bind to a pathogen can be attached to the coating layer. Exemplary virus-binding receptors include, but are not limited to, CD4 (e.g., as a receptor for HIV), CD21 (complement receptor 2, e.g., as a receptor for Epstein-Barr virus), olefactory receptor 1411 (OR14I1) or CD46 (e.g., as receptors for human cytomegalovirus), angiotensin converting enzyme 2 (ACE2, e.g., for SARS-CoV2), and sialic acids or glycoproteins or glycolipids containing sialic acids (e.g., as receptors for influenza viruses). A variety of receptors for bacteria are also known in the art, including, but not limited to, E-cadherin (for Listeria monocytogenes) and beta-1 integrin receptors (for Yersinia), among others^{70,71,72}. These receptors can be conjugated to functionalized lipids in the lipid layer via conjugation methods known in the art, e.g., such as via the reaction of an amine or hydroxyl functionality of the receptor with an activated ester (e.g., a NHS-ester)-functionalized lipid. Alternatively, the receptor can be attached to a lipid in the lipid layer through non-covalent binding interactions of a specific binding pair (e.g., avidin/biotin, nucleic acid/nucleic acid, antigen-antibody, etc.), where one member of the specific binding pair is attached to the receptor and the other member of the specific binding pair is attached to a lipid in the lipid layer.

In some embodiments, pathogen receptor is an ACE2 protein or a pathogen-binding fragment, e.g. virus-binding, fragment thereof. Thus, in some embodiments, the outer surface of the outer layer of the nanoparticle comprises an ACE2 receptor protein or a fragment thereof. In some embodiments, the nanoparticles comprising an ACE2 receptor protein or fragment thereof are referred to herein as a “Nanotrap-ACE2”. In some embodiments, an ACE2 receptor protein or a fragment thereof comprises a recombinant ACE2 receptor protein or fragment thereof. In some embodiments, the ACE2 receptor protein is a human ACE2 receptor protein. Human ACE2 has the amino acid sequence:

MSSSSWLLLSLVAVTAAQSTIEEQAKTFLDKFNHEAEDLFYQSSLASWN
YNTNITEENVQNMNNAGDKWSAFLKEQSTLAQMYPLQEIQNLTVKLQLQ
ALQQNGSSVLSEDKSKRLNTILNTMSTIYSTGKVCNPDNPQECLLLEPG
LNEIMANSLDYNERLWAWESWRSEVGKQLRPLYEEYVVLKNEMARANHY
EDYGDYWRGDYEVNGVDGYDYSRGQLIEDVEHTFEEIKPLYEHLHAYVR
AKLMNAYPSYISPIGCLPAHLLGDMWGRFWTNLYSLTVPFGQKPNIDVT
DAMVDQAWDAQRIFKEAEKFFVSVGLPNMTQGFWENSMLTDPGNVQKAV
CHPTAWDLGKGDFRILMCTKVTMDDFLTAHHEMGHIQYDMAYAAQPFLL
RNGANEGFHEAVGEIMSLSAATPKHLKSIGLLSPDFQEDNETEINFLLK
QALTIVGTLPFTYMLEKWRWMVFKGEIPKDQWMKKWWEMKREIVGVVEP
VPHDETYCDPASLFHVSNDYSFIRYYTRTLYQFQFQEALCQAAKHEGPL
HKCDISNSTEAGQKLENMLRLGKSEPWTLALENVVGAKNMNVRPLLNYF
EPLFTWLKDQNKNSFVGWSTDWSPYADQSIKVRISLKSALGDKAYEWND
NEMYLFRSSVAYAMRQYFLKVKNQMILFGEEDVRVANLKPRISFNFFVT
APKNVSDIIPRTEVEKAIRMSRSRINDAFRLNDNSLEFLGIQPTLGPPN
QPPVSIWLIVFGVVMGVIVVGIVILIFTGIRDRKKKNKARSGENPYASI
DISKGENNPGFQNTDDVQTSF
(NCBI Reference Sequence: NP_068576.1;
SEQ ID NO: 1)

In some embodiments, the ACE2 receptor protein comprises an amino acid sequence comprising amino acids 18-740 (i.e., Gln18-Ser740) of the amino acid sequence of SEQ ID NO: 1, or a substantially similar protein (e.g., a protein having an amino acid sequence having at least 75%, 80%, 85%, 90%, 95%, 96%, 97%, 98%, or 99% homology to the amino acid sequence comprising amino acids 18-740 of SEQ ID NO: 1). The ACE2 receptor protein can be modified with one member of a specific binding pair (i.e., a pair of molecules that specifically bind to one another via covalent or non-covalent bonds, e.g., avidin/biotin or desthiobiotin, an antibody/antigen pair, etc.) and outer layer (b) can comprise a lipid modified with the other member of the specific binding pair. For example, in some embodiments, (c) comprises a streptavidin-modified ACE2 receptor protein and the outer layer comprises a biotin-modified lipid. Thus, the streptavidin-modified ACE2 receptor is attached to the surface of the outer layer via biotin-streptavidin non-covalent binding interactions. Alternatively, the outer layer (b) can comprise a lipid modified with a group (e.g., an activated ester) that can be covalently attached to the ACE2 receptor protein.

In some embodiments, outer layer (c) comprises a pathogen-binding antibody or an antigen-binding fragment thereof. In some embodiments, the pathogen-binding antibody is a virus-binding antibody. In some embodiments, the virus-binding antibody is a virus-neutralizing antibody and outer layer (c) comprises a virus-neutralizing antibody or an antigen-binding fragment thereof. A nanoparticle comprising an antibody (e.g., a virus-neutralizing antibody or antigen-binding fragment thereof) can be referred to herein as a “Nanotrap-Antibody”. The antibody or antibody fragment (e.g., the virus-neutralizing antibody or antigen-binding fragment thereof) can be attached to outer layer (b) via a binding partner interaction (e.g., wherein the virus-neutralizing antibody or fragment thereof is modified by one binding partner of a pair of binding partners and wherein a lipid in outer layer (b) is modified by the other partner of the pair of binding partners). In some embodiments, the antibody or antibody fragment (e.g., the virus-neutralizing antibody or antigen-binding fragment thereof) is covalently attached to a lipid in the outer layer. In some embodiments, the antibody or antibody fragment (e.g., the virus-neutralizing antibody or antigen-binding fragment thereof) is covalently attached to the lipid via reaction with an active ester or imidoester of a functionalized lipid in the lipid layer. In some embodiments, the active ester is an N-hydroxysuccinimide (NHS) ester. However, a variety of other conjugation methods/reactions known in the field of protein conjugation can also be used, as would be apparent to one of ordinary skill in the art upon review of the instant disclosure.

In some embodiments, the virus-neutralizing antibody or antigen-binding fragment thereof is a coronavirus-neutralizing antibody or antigen-binding fragment thereof. In some embodiments, the coronavirus-neutralizing antibody or antigen-binding fragment thereof is a SARS-CoV-2-neutralizing antibody or antigen-binding fragment thereof. SARS-CoV-2 neutralizing antibodies are known in the art¹⁰⁻¹⁴ and can be obtained from commercial sources (e.g., catalog number 40592-MM57 (RRID: AB_2857935), catalog number 40591-MM31 (RRID: AB_2857934), or catalog number 40592-R001 (RRID: AB_2857936) from SinoBiological (Beijing, China); antibody 12D3 from ThermoFisher Scientific (Waltham, Massachusetts, United States of America); catalog number A19215 (RRID:AB_2862633) from ABclonal (Woburn, Massachusetts, United States of America); or catalog number CABT-CS064 (RRID:AB_2891088), among others. As additional examples, SARS-CoV-2-neutralizing antibodies targeting the N terminal domain (NTD) of SARS-COV2 have been reported, including, but not limited to COV2-2676 and COV2-2489, which bind to a common antigenic site on the NTD and inhibit pseudotyped and authentic SARS-CoV-2 infection in vitro; NTD-targeting nAbs 159, BLN12, and BLN14; 89C8, which has been found to neutralize pseudotyped SARS-CoV-2 infection with an IC₅₀ of 4.5 nM; nAbs BLN1, BLN12, and P008_056, have been shown to neutralize authentic SARS-CoV-2 with IC₅₀ values of 8, 8, and 14 ng/ml, respectively; and S2L28, S2M28, S2X28, and S2X333, which have been found to potently neutralize infection of both pseudotyped and live SARS-CoV-2 with an IC₅₀ value as low as 2 ng/ml⁶⁹.

Alternatively, binding antibodies, such as neutralizing antibodies, such as SARS-CoV-2 neutralizing antibodies, can be prepared via methods known in the art, e.g., using an antigen comprising a SARS-CoV-2 protein or fragment thereof. In some embodiments, the virus-neutralizing antibody or the antigen-binding fragment thereof is an antibody or antigen-binding fragment thereof that binds to a SARS-CoV-2 spike (S) protein. In some embodiments, the antibody or antigen-binding fragment binds to the S1 subunit of the SARS-CoV-2 spike protein. For example, the amino acid sequence of the SARS-CoV-2 S protein (isolate Wuhan-Hu-1) is:

MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRENGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT
(SEQ ID NO: 2; NCBI Reference
Sequence: YP_009724390.1, SEQ ID NO: 2).

Suitable SARS-CoV-2 antigens can be prepared via recombinant methods known in the art or can be purchased from commercial sources. Proteins or protein fragments comprising amino acid sequences from SARS-CoV-2 variants can also be used. SARS-CoV-2 spike protein variant amino acid sequences include, but are not limited to, SEQ ID NOS: 3-10, as follows:

SEQ ID NO: 3 (ARSCoV2 B.1.1.7 S Protein Variant
amino acid sequence (GENBANK®
Accession No. QTC27506.1)):
MFVFLVLLPLVSSQCVNLTTRTQLPPAYTNSFTRGVYYPDKVFRSSVLH
STQDLFLPFFSNVTWFHAISGTNGTKRFDNPVLPFNDGVYFASTEKSNI
IRGWIFGTTLDSKTQSLLIVNNATNVVIKVCEFQFCNDPFLGVYHKNNK
SWMESEFRVYSSANNCTFEYVSQPFLMDLEGKQGNFKNLREFVFKNIDG
YFKIYSKHTPINLVRDLPQGFSALEPLVDLPIGINITRFQTLLALHRSY
LTPGDSSSGWTAGAAAYYVGYLQPRTFLLKYNENGTITDAVDCALDPLS
ETKCTLKSFTVEKGIYQTSNFRVQPTESIVRFPNITNLCPFGEVENATR
FASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSK
VGGNYNYLYRLFRKSNLKPFERDISTEIYQAGSTPCNGVEGENCYFPLQ
SYGFQPTYGVGYQPYRVVVLSFELLHAPATVCGPKKSTNLVKNKCVNFN
FNGLTGTGVLTESNKKFLPFQQFGRDIDDTTDAVRDPQTLEILDITPCS
FGGVSVITPGTNTSNQVAVLYQGVNCTEVPVAIHADQLTPTWRVYSTGS
NVFQTRAGCLIGAEHVNNSYECDIPIGAGICASYQTQTNSHRRARSVAS
QSIIAYTMSLGAENSVAYSNNSIAIPTNFTISVTTEILPVSMTKTSVDC
TMYICGDSTECSNLLLQYGSFCTQLNRALTGIAVEQDKNTQEVFAQVKQ
IYKTPPIKDFGGFNFSQILPDPSKPSKRSFIEDLLFNKVTLADAGFIKQ
YGDCLGDIAARDLICAQKFNGLTVLPPLLTDEMIAQYTSALLAGTITSG
WTFGAGAALQIPFAMQMAYRFNGIGVTQNVLYENQKLIANQFNSAIGKI
QDSLSSTASALGKLQDVVNQNAQALNTLVKQLSSNFGAISSVLNDILSR
LDKVEAEVQIDRLITGRLQSLQTYVTQQLIRAAEIRASANLAATKMSEC
VLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVI
GIVNNTVYDPLQPELDSFKEELDKYFKNHTSPDVDLGDISGINASVVNI
QKEIDRLNEVAKNLNESLIDLQELGKYEQYIKWPWYIWLGFIAGLIAIV
MVTIMLCCMTSCCSCLKGCCSCGSCCKFDEDDSEPVLKGVKLHYT
SEQ ID NO: 4 (SARSCoV2 B.1.1.7E484K S Protein
Variant amino acid sequence):
MFVFLVLLPLVSSQCVNLTTRTQLPPAYTNSFTRGVYYPDKVFRSSVLH
STQDLFLPFFSNVTWFHAISGTNGTKRFDNPVLPFNDGVYFASTEKSNI
IRGWIFGTTLDSKTQSLLIVNNATNVVIKVCEFQFCNDPFLGVYHKNNK
SWMESEFRVYSSANNCTFEYVSQPFLMDLEGKQGNFKNLREFVFKNIDG
YFKIYSKHTPINLVRDLPQGFSALEPLVDLPIGINITRFQTLLALHRSY
LTPGDSSSGWTAGAAAYYVGYLQPRTFLLKYNENGTITDAVDCALDPLS
ETKCTLKSFTVEKGIYQTSNFRVQPTESIVRFPNITNLCPFGEVENATR
FASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSK
VGGNYNYLYRLFRKSNLKPFERDISTEIYQAGSTPCNGVKGFNCYFPLQ
SYGFQPTYGVGYQPYRVVVLSFELLHAPATVCGPKKSTNLVKNKCVNFN
FNGLTGTGVLTESNKKFLPFQQFGRDIDDTTDAVRDPQTLEILDITPCS
FGGVSVITPGTNTSNQVAVLYQGVNCTEVPVAIHADQLTPTWRVYSTGS
NVFQTRAGCLIGAEHVNNSYECDIPIGAGICASYQTQTNSHRRARSVAS
QSIIAYTMSLGAENSVAYSNNSIAIPTNFTISVTTEILPVSMTKTSVDC
TMYICGDSTECSNLLLQYGSFCTQLNRALTGIAVEQDKNTQEVFAQVKQ
IYKTPPIKDFGGFNFSQILPDPSKPSKRSFIEDLLFNKVTLADAGFIKQ
YGDCLGDIAARDLICAQKFNGLTVLPPLLTDEMIAQYTSALLAGTITSG
WTFGAGAALQIPFAMQMAYRENGIGVTQNVLYENQKLIANQFNSAIGKI
QDSLSSTASALGKLQDVVNQNAQALNTLVKQLSSNFGAISSVLNDILSR
LDKVEAEVQIDRLITGRLQSLQTYVTQQLIRAAEIRASANLAATKMSEC
VLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVI
GIVNNTVYDPLQPELDSFKEELDKYFKNHTSPDVDLGDISGINASVVNI
QKEIDRLNEVAKNLNESLIDLQELGKYEQYIKWPWYIWLGFIAGLIAIV
MVTIMLCCMTSCCSCLKGCCSCGSCCKFDEDDSEPVLKGVKLHYT
SEQ ID NO: 5 (SARSCoV2 B.1.1.7S494P S
Protein Variant amino acid sequence):
MFVFLVLLPLVSSQCVNLTTRTQLPPAYTNSFTRGVYYPDKVFRSSVLH
STQDLFLPFFSNVTWFHAISGTNGTKRFDNPVLPFNDGVYFASTEKSNI
IRGWIFGTTLDSKTQSLLIVNNATNVVIKVCEFQFCNDPFLGVYHKNNK
SWMESEFRVYSSANNCTFEYVSQPFLMDLEGKQGNFKNLREFVFKNIDG
YFKIYSKHTPINLVRDLPQGFSALEPLVDLPIGINITRFQTLLALHRSY
LTPGDSSSGWTAGAAAYYVGYLQPRTFLLKYNENGTITDAVDCALDPLS
ETKCTLKSFTVEKGIYQTSNFRVQPTESIVRFPNITNLCPFGEVFNATR
FASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSK
VGGNYNYLYRLFRKSNLKPFERDISTEIYQAGSTPCNGVKGFNCYFPLQ
PYGFQPTYGVGYQPYRVVVLSFELLHAPATVCGPKKSTNLVKNKCVNFN
FNGLTGTGVLTESNKKFLPFQQFGRDIDDTTDAVRDPQTLEILDITPCS
FGGVSVITPGTNTSNQVAVLYQGVNCTEVPVAIHADQLTPTWRVYSTGS
NVFQTRAGCLIGAEHVNNSYECDIPIGAGICASYQTQTNSHRRARSVAS
QSIIAYTMSLGAENSVAYSNNSIAIPTNFTISVTTEILPVSMTKTSVDC
TMYICGDSTECSNLLLQYGSFCTQLNRALTGIAVEQDKNTQEVFAQVKQ
IYKTPPIKDFGGFNFSQILPDPSKPSKRSFIEDLLFNKVTLADAGFIKQ
YGDCLGDIAARDLICAQKFNGLTVLPPLLTDEMIAQYTSALLAGTITSG
WTFGAGAALQIPFAMQMAYRFNGIGVTQNVLYENQKLIANQFNSAIGKI
QDSLSSTASALGKLQDVVNQNAQALNTLVKQLSSNFGAISSVLNDILSR
LDKVEAEVQIDRLITGRLQSLQTYVTQQLIRAAEIRASANLAATKMSEC
VLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVI
GIVNNTVYDPLQPELDSFKEELDKYFKNHTSPDVDLGDISGINASVVNI
QKEIDRLNEVAKNLNESLIDLQELGKYEQYIKWPWYIWLGFIAGLIAIV
MVTIMLCCMTSCCSCLKGCCSCGSCCKFDEDDSEPVLKGVKLHYT
SEQ ID NO: 6 (SARSCoV2 P.la S Protein Variant
amino acid sequence):
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVEN
ATRFASVYAWNRKRISNCVADYSVLYNSASFSTFKCYGVSPTKLNDLCF
TNVYADSFVIRGDEVRQIAPGQTGNIADYNYKLPDDFTGCVIAWNSNNL
DSKVGGNYNYLYRLFRKSNLKPFERDISTEIYQAGSTPCNGVKGFNCYF
PLQSYGFQPTYGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQGVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT
SEQ ID NO: 7 (SARSCoV2 P.1b S Protein Variant
amino acid sequence):
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGTIADYNYKLPDDFTGCVIAWNSNNL
DSKVGGNYNYLYRLFRKSNLKPFERDISTEIYQAGSTPCNGVKGFNCYF
PLQSYGFQPTYGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQGVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT
SEQ ID NO: 8 (SARSCoV2 B.1.351 S Protein Variant
amino acid sequence):
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVEN
ATRFASVYAWNRKRISNCVADYSVLYNSASFSTFKCYGVSPTKLNDLCF
TNVYADSFVIRGDEVRQIAPGQTGNIADYNYKLPDDFTGCVIAWNSNNL
DSKVGGNYNYLYRLFRKSNLKPFERDISTEIYQAGSTPCNGVKGFNCYF
PLQSYGFQPTYGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQGVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRENGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT
SEQ ID NO: 9 (SARSCoV2 B.1.427 S Protein Variant
amino acid sequence (GENBANK®
Accession No. QQX31457.1)):
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYRYRLFRKSNLKPFERDISTEIYQAGSTPCNGVEGENCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQGVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKENGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYTand
SEQ ID NO: 10 (SARSCoV2 B.1.429 S Protein Variant
amino acid sequence (GENBANK®
Accession No. QPJ72086.1)):
MFVFLVLLPLVSIQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSCMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVEN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYRYRLFRKSNLKPFERDISTEIYQAGSTPCNGVEGENCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQGVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT

The SARS-CoV-2 spike protein comprises two domains: S1 (amino acids 14-685 of SEQ ID NO:2) and S2 (amino acids 686-1273 of SEQ ID NO: 2). The S1 domain includes the receptor binding domain (RBD, amino acids 319-541 of SEQ ID NO: 2) responsible for binding to ACE2. The S1 domain also includes a N-terminal domain (NTD, amino acids 13-304 of SEQ ID NO: 2). In some embodiments, the SARS-CoV-2 neutralizing antibody or antigen-binding fragment binds to the S1 domain of a SARS-CoV-2 spike protein (e.g., an amino acid sequence comprising amino acids 14-685 of SEQ ID NO: 2 or an amino acid sequence having at least 70%, 75%, 80%, 85%, 90%, or 95% homology thereto). In some embodiments, the SARS-CoV-2 neutralizing antibody or antigen-binding fragment binds to the RBD of a SARS-CoV-2 spike protein (e.g., an amino acid sequence comprising amino acids 319-541 of SEQ ID NO: 2 or an amino acid sequence having at least about 70% (e.g., at least about 70%, about 75%, about 80%, about 85%, or about 90%) homology thereto. In some embodiments, the SARS-CoV-2 antibody or antigen-binding fragment thereof binds to the S1 domain of one of SEQ ID Nos: 3-10. In some embodiments, the SARS-CoV-2 antibody or antigen-binding fragment thereof binds to the RBD domain of one of SEQ ID Nos: 3-10.

SARS-CoV-2 antigens (e.g., comprising the nucleocapsid protein, the spike protein S1 domain, the spike protein S2 domain, the spike protein S1+S2 domain, and/or the spike protein receptor binding domain (RBD) can be purchased, for example, from Sigma Aldrich (St. Louis, Missouri, United States of America) or AcroBiosystems (Newark, Delaware, United States of America; e.g., catalog number SPN-C52H7 for spike protein and NUN-C5227 for nucleocapsid protein). In some embodiments, the SARS-CoV-2 neutralizing antibody is prepared using an antigen comprising one of SEQ ID NOS: 2-10, the S1 or RBD domain thereof, or an amino acid sequence having at least 70%, 75%, 80%, 85%, 90%, or 95% homology thereto.

In some embodiments, the virus-binding antibody or antigen-binding fragment thereof, e.g. the virus-neutralizing antibody or antigen binding fragment thereof, binds to a spike protein of another coronavirus, such as the spike proteins of OC43-CoV, HKU1-CoV, NL63-CoV, and 229E-CoV are as follows:

OC43-CoV spike protein (SEQ ID NO: 11):
MFLILLISLPTAFAVIGDLKCTSDNINDKDTGPPPISTDTVDVTNGLGT
YYVLDRVYLNTTLFLNGYYPTSGSTYRNMALKGSVLLSRLWFKPPFLSD
FINGIFAKVKNTKVIKDRVMYSEFPAITIGSTFVNTSYSVVVQPRTINS
TQDGDNKLQGLLEVSVCQYNMCEYPQTICHPNLGNHRKELWHLDTGVVS
CLYKRNFTYDVNADYLYFHFYQEGGTFYAYFTDTGVVTKFLFNVYLGMA
LSHYYVMPLTCNSKLTLEYWVTPLTSRQYLLAFNQDGIIFNAVDCMSDF
MSEIKCKTQSIAPPTGVYELNGYTVQPIADVYRRKPNLPNCNIEAWLND
KSVPSPLNWERKTFSNCNFNMSSLMSFIQADSFTCNNIDAAKIYGMCFS
SITIDKFAIPNGRKVDLQLGNLGYLQSFNYRIDTTATSCQLYYNLPAAN
VSVSRFNPSTWNKRFGFIEDSVFKPRPAGVLTNHDVVYAQHCFKAPKNF
CPCKLNGSCVGSGPGKNNGIGTCPAGTNYLTCDNLCTPDPITFTGTYKC
PQTKSLVGIGEHCSGLAVKSDYCGGNSCTCRPQAFLGWSADSCLQGDKC
NIFANFILHDVNSGLTCSTDLQKANTDIILGVCVNYDLYGILGQGIFVE
VNATYYNSWQNLLYDSNGNLYGFRDYITNRTFMIRSCYSGRVSAAFHAN
SSEPALLFRNIKCNYVFNNSLTRQLQPINYFDSYLGCVVNAYNSTAISV
QTCDLTVGSGYCVDYSKNRRSRGAITTGYRFTNFEPFTVNSVNDSLEPV
GGLYEIQIPSEFTIGNMVEFIQTSSPKVTIDCAAFVCGDYAACKSQLVE
YGSFCDNINAILTEVNELLDTTQLQVANSLMNGVTLSTKLKDGVNFNVD
DINFSPVLGCLGSECSKASSRSAIEDLLFDKVKLSDVGFVEAYNNCTGG
AEIRDLICVQSYKGIKVLPPLLSENQISGYTLAATSASLFPPWTAAAGV
PFYLNVQYRINGLGVTMDVLSQNQKLIANAFNNALYAIQEGFDATNSAL
VKIQAVVNANAEALNNLLQQLSNRFGAISASLQEILSRLDALEAEAQID
RLINGRLTALNAYVSQQLSDSTLVKFSAAQAMEKVNECVKSQSSRINFC
GNGNHIISLVQNAPYGLYFIHFSYVPTKYVTARVSPGLCIAGDRGIAPK
SGYFVNVNNTWMYTGSGYYYPEPITENNVVVMSTCAVNYTKAPYVMLNT
SIPNLPDFKEELDQWFKNQTSVAPDLSLDYINVTFLDLQVEMNRLQEAI
KVLNQSYINLKDIGTYEYYVKWPWYVWLLICLAGVAMLVLLFFICCCTG
CGTSCFKKCGGCCDDYTGYQELVIKTSHDD
HKU1-CoV spike protein (SEQ ID NO. 12):
MLLIIFILPTTLAVIGDFNCTNFAINDLNTTVPRISEYVVDVSYGLGTY
YILDRVYLNTTILFTGYFPKSGANFRDLSLKGTTYLSTLWYQKPFLSDF
NNGIFSRVKNTKLYVNKTLYSEFSTIVIGSVFINNSYTIVVQPHNGVLE
ITACQYTMCEYPHTICKSKGSSRNESWHFDKSEPLCLFKKNFTYNVSTD
WLYFHFYQERGTFYAYYADSGMPTTFLFSLYLGTLLSHYYVLPLTCNAI
SSNTDNETLQYWVTPLSKRQYLLKFDNRGVITNAVDCSSSFFSEIQCKT
KSLLPNTGVYDLSGFTVKPVATVHRRIPDLPDCDIDKWLNNFNVPSPLN
WERKIFSNCNFNLSTLLRLVHTDSFSCNNFDESKIYGSCFKSIVLDKFA
IPNSRRSDLQLGSSGFLQSSNYKIDTTSSSCQLYYSLPAINVTINNYNP
SSWNRRYGFNNFNLSSHSVVYSRYCFSVNNTFCPCAKPSFASSCKSHKP
PSASCPIGTNYRSCESTTVLDHTDWCRCSCLPDPITAYDPRSCSQKKS
LVGVGEHCAGFGVDEEKCGVLDGSYNVSCLCSTDAFLGWSYDTCVSNNRC
NIFSNFILNGINSGTTCSNDLLQPNTEVFTDVCVDYDLYGITGQGIFKE
VSAVYYNSWQNLLYDSNGNIIGFKDFVTNKTYNIFPCYAGRVSAAFHQN
ASSLALLYRNLKCSYVLNNISLTTQPYFDSYLGCVFNADNLTDYSVSSC
ALRMGSGFCVDYNSPSSSSSRRKRRSISASYRFVTFEPFNVSFVNDSIE
SVGGLYEIKIPTNFTIVGQEEFIQTNSPKVTIDCSLFVCSNYAACHDLL
SEYGTFCDNINSILDEVNGLLDTTQLHVADTLMQGVTLSSNLNTNLHFD
VDNINFKSLVGCLGPHCGSSSRSFFEDLLFDKVKLSDVGFVEAYNNCTG
GSEIRDLLCVQSFNGIKVLPPILSESQISGYTTAATVAAMFPPWSAAAG
IPFSLNVQYRINGLGVTMDVLNKNQKLIATAFNNALLSIQNGFSATNSA
LAKIQSVVNSNAQALNSLLQQLFNKFGAISSSLQEILSRLDALEAQVQI
DRLINGRLTALNAYVSQQLSDISLVKFGAALAMEKVNECVKSQSPRINF
CGNGNHILSLVQNAPYGLLFMHFSYKPISFKTVLVSPGLCISGDVGIAP
KQGYFIKHNDHWMFTGSSYYYPEPISDKNVVFMNTCSVNFTKAPLVYLN
HSVPKLSDFESELSHWFKNQTSIAPNLTLNLHTINATFLDLYYEMNLIQ
ESIKSLNNSYINLKDIGTYEMYVKWPWYVWLLISFSFIIFLVLLFFICC
CTGCGSACFSKCHNCCDEYGGHHDFVIKTSHDD
NL63-CoV spike protein (SEQ ID NO. 13):
MKLFLILLVLPLASCFFTCNSNANLSMLQLGVPDNSSTIVTGLLPTHWF
CANQSTSVYSANGFFYIDVGNHRSAFALHTGYYDANQYYIYVTNEIGLN
ASVTLKICKFSRNTTFDFLSNASSSFDCIVNLLFTEQLGAPLGITISGE
TVRLHLYNVTRTFYVPAAYKLTKLSVKCYFNYSCVFSVVNATVTVNVTT
HNGRVVNYTVCDDCNGYTDNIFSVQQDGRIPNGFPFNNWFLLTNGSTLV
DGVSRLYQPLRLTCLWPVPGLKSSTGFVYFNATGSDVNCNGYQHNSVVD
VMRYNLNFSANSLDNLKSGVIVFKTLQYDVLFYCSNSSSGVLDTTIPFG
PSSQPYYCFINSTINTTHVSTFVGILPPTVREIVVARTGQFYINGFKYF
DLGFIEAVNFNVTTASATDFWTVAFATFVDVLVNVSATNIQNLLYCDSP
FEKLQCEHLQFGLQDGFYSANFLDDNVLPETYVALPIYYQHTDINFTAT
ASFGGSCYVCKPHQVNISLNGNTSVCVRTSHFSIRYIYNRVKSGSPGDS
SWHIYLKSGTCPFSFSKLNNFQKFKTICFSTVEVPGSCNFPLEATWHYT
SYTIVGALYVTWSEGNSITGVPYPVSGIREFSNLVLNNCTKYNIYDYVG
TGIIRSSNQSLAGGITYVSNSGNLLGFKNVSTGNIFIVTPCNQPDQVAV
YQQSIIGAMTAVNESRYGLQNLLQLPNFYYVSNGGNNCTTAVMTYSNFG
ICADGSLIPVRPRNSSDNGISAIITANLSIPSNWTTSVQVEYLQITSTP
IVVDCATYVCNGNPRCKNLLKQYTSACKTIEDALRLSAHLETNDVSSML
TFDSNAFSLANVTSFGDYNLSSVLPQRNIRSSRIAGRSALEDLLFSKVV
TSGLGTVDVDYKSCTKGLSIADLACAQYYNGIMVLPGVADAERMAMYTG
SLIGGMVLGGLTSAAAIPFSLALQARLNYVALQTDVLQENQKILAASFN
KAINNIVASFSSVNDAITQTAEAIHTVTIALNKIQDVVNQQGSALNHLT
SQLRHNFQAISNSIQAIYDRLDSIQADQQVDRLITGRLAALNAFVSQVL
NKYTEVRGSRRLAQQKINECVKSQSNRYGFCGNGTHIFSIVNSAPDGLL
FLHTVLLPTDYKNVKAWSGICVDGIYGYVLRQPNLVLYSDNGVFRVTSR
VMFQPRLPVLSDFVQIYNCNVTFVNISRVELHTVIPDYVDVNKTLQEFA
QNLPKYVKPNFDLTPFNLTYLNLSSELKQLEAKTASLFQTTVELQGLID
QINSTYVDLKLLNRFENYIKWPWWVWLIISVVFVVLLSLLVFCCLSTGC
CGCCNCLTSSMRGCCDCGSTKLPYYEFEKVHVQ
229E-CoV spike protein (SEQ ID NO: 14):
MFVLLVAYALLHIAGCQTTNGLNTSYSVCNGCVGYSENVFAVESGGYIP
SDFAFNNWFLLTNTSSVVDGVVRSFQPLLLNCLWSVSGLRFTTGFVYFN
GTGRGDCKGFSSDVLSDVIRYNLNFEENLRRGTILFKTSYGVVVFYCTN
NTLVSGDAHIPFGTVLGNFYCFVNTTIGNETTSAFVGALPKTVREFVIS
RTGHFYINGYRYFTLGNVEAVNFNVTTAETTDFCTVALASYADVLVNVS
QTSIANIIYCNSVINRLRCDQLSFDVPDGFYSTSPIQSVELPVSIVSLP
VYHKHTFIVLYVDFKPQSGGGKCFNCYPAGVNITLANFNETKGPLCVDT
SHFTTKYVAVYANVGRWSASINTGNCPFSFGKVNNFVKFGSVCFSLKDI
PGGCAMPIVANWAYSKYYTIGSLYVSWSDGDGITGVPQPVEGVSSFMNV
TLDKCTKYNIYDVSGVGVIRVSNDTFLNGITYTSTSGNLLGFKDVTKGT
IYSITPCNPPDQLVVYQQAVVGAMLSENFTSYGFSNVVELPKFFYASNG
TYNCTDAVLTYSSFGVCADGSIIAVQPRNVSYDSVSAIVTANLSIPSNW
TTSVQVEYLQITSTPIVVDCSTYVCNGNVRCVELLKQYTSACKTIEDAL
RNSARLESADVSEMLTFDKKAFTLANVSSFGDYNLSSVIPSLPTSGSRV
AGRSAIEDILFSKLVTSGLGTVDADYKKCTKGLSIADLACAQYYNGIMV
LPGVADAERMAMYTGSLIGGIALGGLTSAVSIPFSLAIQARLNYVALQT
DVLQENQKILAASFNKAMTNIVDAFTGVNDAITQTSQALQTVATALNKI
QDVVNQQGNSLNHLTSQLRQNFQAISSSIQAIYDRLDTIQADQQVDRLI
TGRLAALNVFVSHTLTKYTEVRASRQLAQQKVNECVKSQSKRYGFCGNG
THIFSIVNAAPEGLVFLHTVLLPTQYKDVEAWSGLCVDGTNGYVLRQPN
LALYKEGNYYRITSRIMFEPRIPTMADFVQIENCNVTFVNISRSELQTI
VPEYIDVNKTLQELSYKLPNYTVPDLVVEQYNQTILNLTSEISTLENKS
AELNYTVQKLQTLIDNINSTLVDLKWLNRVETYIKWPWWVWLCISVVLI
FVVSMLLLCCCSTGCCGFFSCFASSIRGCCESTKLPYYDVEKIHIQ

Thus, in some embodiments, the pathogen-binding antibody or antigen-binding fragment thereof, e.g., the virus-neutralizing antibody or antigen-binding fragment thereof, binds to an amino acid sequence comprising one of SEQ ID NOS. 11-14; or a fragment thereof. In some embodiments, the virus-neutralizing antibody or antigen-binding fragment thereof binds to an antigen that comprises or consists of an amino acid sequence having at least about 70%, about 75%, about 80%, about 85%, or about 90% homology to the amino acid sequence of one of the group comprising SEQ ID NOS: 11-14, or a fragment thereof. In some embodiments, the virus-neutralizing antibody or antigen-binding fragment thereof is prepared using an antigen comprising or consists of an amino acid sequence having at least about 95% homology (e.g., at least about 95%, about 96%, about 97%, about 98%, or about 99% homology) to the amino acid sequence of one of SEQ ID NOS: 11-14, or a fragment thereof.

However, the pathogen-binding antibody or antigen-binding fragment thereof is not limited to antibodies and antibody fragments that bind (e.g., neutralize) SARS-CoV-2 or another coronavirus, but can also include antibodies and antibody fragment that bind other pathogens, such as other viruses (e.g., viruses of the families herpesviridae, retroviridae, papillomaviridae, caliciviridae, or picornaviridae) or bacteria. In some embodiments, the virus is another respiratory virus, e.g., influenza virus, respiratory syncytial virus (RSV), paramyxovirus, adenovirus, parainfluenza virus (PIV), bocavirus, metapneumovirus, orthopneumovirus, enterovirus, rhinovirus, and the like. In some embodiments, the virus is selected from human immunodeficiency virus (HIV), hepatitis B virus (HBV), dengue virus, and Zika virus (ZIKV). Broadly neutralizing antibodies (bNAbs) against HIV-1 have recently been described for preventing or treating HIV-1. These bNAbs include, for example, b12, 447-52D, 2G12, 17b, 2F5, 4E10 and Z13, with different specificities⁶⁶. Broadly neutralizing antibodies (bNAbs) to the HBV S antigen (HBsAg) have also been described⁶⁷. For ZIKV, neutralizing antibodies that bind to the E proteins on the surface of the viral particle have been described⁶⁸. Antibodies that bind to bacteria, e.g., by targeting bacterial exotoxins, bacterial cell surface proteins, bacterial cell surface polysaccharides, or other bacterial antigen, are also known in the art⁷³.

The phagocyte-specific ligand (d) can comprise a moiety that targets any phagocytic cell, such as, but not limited to a macrophage, a dendritic cell, a neutrophil, a monocyte, or a mast cell. Suitable phagocyte-specific ligands include, but are not limited to, fibronectin, vitronectin, mannan, lipopolysaccharide binding protein, and phosphoserine-containing compounds. In some embodiments, the ligand that targets a phagocytic cell comprises a phosphoserine group. In some embodiments, the phagocyte-specific ligand is attached (e.g., covalently attached) to a lipid in layer (b). In some embodiments, the phagocyte-specific ligand (d) is part of a lipid in layer (b). For example, in some embodiments, outer layer (b) comprises one or more phosphatidylserine lipid selected from the group comprising 1,2-dioleoyl-sn-glycero-3-phospho-L-serine (DOPS), 1,2-distearoyl-sn-glycero-3-phosphatidylserine (DSPS), 1;1-palmitoyl-2-oleoyl-sn-glycero-3-phospho-L-serine (POPS), 1,2-dipalmitoyl-sn-glycero-3-phosphoserine (DPPS), and 1,2-ditetradecanoyl-sn-glycero-3-phospho-L-serine (DMPS); and the phagocyte-specific ligand (d) comprises the phosphoserine moiety from the one or more phosphatidylserine lipid. In some embodiments, the lipid layer (b) comprises up to about 25% of the one or more phosphatidylserine lipids. In some embodiments, lipid layer (b) comprises between about 5% and about 20% or one or more phosphatidylserine lipids (e.g., about 5, 6, 7, 8, 9, 10, 11, 12, 13, 14, 15, 16, 17, 18, 19, or about 20% of one or more phosphatidylserine lipids, wherein the percentage is a molar percentage compared to the total number of moles of lipid in layer (b)). In some embodiments, lipid layer (b) comprises about 10% to about 15% of the one or more phosphatidylserine lipids. In some embodiments, lipid layer (b) comprises about 15% of the one or more phosphatidylserine lipid. However, higher amounts of phosphatidylserine lipids can also be used. Thus, in some embodiments, the lipid layer comprises about 5% to about 70% (e.g., 5%, 10%, 15%, 20%, 25%, 30%, 35%, 40%, 45%, 50%, 55%, 60%, 65%, or about 70%) of one or more phosphatidylserine lipids.

Outer layer (b) can also comprise one or more additional lipids. In some embodiments, the outer layer comprises one or more lipids of the group comprising 1,2-distearoyl-sn-glycero-3-phosphocholine (DSPC), cholesterol, and a poly(ethylene glycol) (PEG)-modified lipid. For example, in some embodiments, the lipid layer can comprise 0% to about 50% cholesterol (e.g., 0%, 5%, 10%, 15%, 20%, 25% 30%, 35%, 40%, 45%, or about 50% cholesterol). In some embodiments, the PEG-modified lipid is 1,2-distearoyl-sn-glycero-3-phosphoethanolamine (DSPE)-mPEG₂₀₀₀. In some embodiments, the lipid layer can comprise about 1% to about 50% (e.g., about 1%, 5%, 10%, 15%, 20%, 25%, 30%, 40%, 45%, or about 50%) of one or more PEG-modified lipids (e.g., DSPE-mPEG₂₀₀₀). In some embodiments, lipid layer (b) comprises about 50% to about 65% DSPC (e.g., 50%, 51%, 52%, 53%, 54%, 55%, 56%, 57%, 58%, 59%, 60%, 61%, 62%, 63%, 64%, or about 65% DSPC; again based on the total number of moles of lipid in the lipid layer). In some embodiments, lipid layer (b) comprises about 30% cholesterol. In some embodiments, lipid layer (b) comprises about 4.5% DSPE-mPEG₂₀₀₀. However, other lipids (e.g., with different chain lengths, saturated/unsaturated bonds, charges, solubilities) could also be used in addition to, or in place of, one of these lipids. For example, in some embodiments, the lipid layer can include one or more phosphatidyl choline lipids (e.g., DOPC, DSPC, DPPC, DMPC, DLPC, and POPC), phosphatidyl glycerol lipids (e.g., DLPG, DMPG, DPPG, DSPG, DOPG, and POPG), phosphatidic acid lipids (e.g., DLPA, DMPA, DPPA, DSPA, DOPA, and POPA), and/or phosphatidyl ethanolamine lipids (e.g., DLPE, DMPE, DPPE, DSPE, DOPE, and POPE).

The lipid layer can further comprise a lipid that is attached or is functionalized for attachment to a pathogen-binding receptor (e.g., an ACE2 receptor protein or fragment thereof) or a pathogen-binding antibody or an antigen-binding fragment thereof (e.g., a virus-neutralizing antibody or an antigen-binding fragment thereof). The attachment can be covalent or non-covalent (e.g., hydrogen bonding, ionic interactions, etc). In some embodiments, the attachment is through binding of a specific binding pair. Thus, in some embodiments, prior to attachment to the pathogen-binding receptor (e.g., ACE2 receptor protein or fragment thereof) or the pathogen-binding antibody or antigen-binding fragment thereof (e.g., virus-neutralizing antibody or fragment thereof), the lipid layer can comprise, for instance, an N-hydroxysuccinimide (NHS)-modified lipid (e.g., DSPE-PEG₂₀₀₀-NHS) or a biotin- or streptavidin-modified lipid (e.g., DSPE-PEG₂₀₀₀-biotin). Following modification with the pathogen-binding receptor (e.g., ACE2 protein of fragment thereof) or pathogen-binding antibody or antigen-binding fragment thereof (e.g., virus-neutralizing antibody or antigen-binding fragment thereof), the NHS-modified lipid can be transformed, for example, into DSPE-PEG₂₀₀₀-Antibody (or antibody fragment) and the biotin-modified lipid can be transformed, for example, to DSPE-PEG₂₀₀₀-biotin-streptavidin (SA)-receptor (e.g., DSPE-PEG2000-biotin-SA-ACE2). In some embodiments, the lipid layer comprises about 0.1% to about 10% of a lipid that is functionalized for attachment and/or attached to a pathogen-binding receptor (e.g., ACE2 protein or fragment thereof) and/or with a pathogen-binding antibody (e.g., virus-neutralizing antibody or antigen-binding fragment thereof), such as a NHS-modified or other active ester-modified lipid or a biotin-modified lipid. In some embodiments, the lipid layer comprises about 0.2% to about 2% of a biotin-containing lipid (e.g., about 0.2, 0.4, 0.6, 0.8, 1.0, 1.2, 1.4, 1.6, 1.8, or about 2.0% of a biotin-containing lipid. In some embodiments, the lipid layer comprises about 0.5% of a biotin-containing lipid. In some embodiments, the lipid layer comprises about 2% to about 10% (e.g., about 2, 3, 4, 5, 6, 7, 8, 9, or 10%) of a lipid comprising a NHS ester group or a linkage (e.g., an amide linkage) that resulted from reacting an NHS ester group with an amine (e.g., an amine group present on a protein, such as a virus-neutralizing antibody). In some embodiments, the lipid layer comprises about 5% of a lipid comprising a NHS ester group or a linkage that resulted from reacting an NHS ester group with an amine.

In some embodiments, the presently disclosed subject matter provides a pharmaceutical formulation comprising a nanoparticle of the presently disclosed subject matter and a pharmaceutically acceptable carrier. In some embodiments, the pharmaceutical formulation is formulated for intranasal administration (e.g., as an intranasal spray or aerosol). In some embodiments, the pharmaceutical formulation is formulated for oral administration. In some embodiments, the pharmaceutical formulation is formulated as a liquid, e.g., for oral, ocular, subcutaneous, intramuscular, or intravenous injection. In some embodiments, the presently disclosed nanoparticles or the pharmaceutical formulations thereof are provided for use in treating or preventing a pathogen infection (e.g., a bacterial or viral infection, such as a SARS-CoV-2 infection).

In some embodiments, the presently disclosed subject matter provides a method for treating or preventing a pathogen infection in a subject in need of treatment or prevention thereof wherein the method comprises administering to the subject a functionalized nanoparticle of the presently disclosed subject matter (e.g., a Nanotrap) or a pharmaceutical formulation thereof. The pathogen infection can be, for example, any bacterial or viral infection, including both DNA viruses or RNA viruses. In some embodiments, the pathogen infection is a viral infection. In some embodiments, the viral infection is an infection of a virus of the family coronaviridae, herpesviridae, retroviridae, papillomaviridae, caliciviridae, or picornaviridae. In some embodiments, the viral infection is an infection of a respiratory virus, such as, but not limited to, coronavirus, influenza virus, respiratory syncytial virus (RSV), paramyxovirus, adenovirus, parainfluenza virus (PIV), bocavirus, metapneumovirus, orthopneumovirus, enterovirus, rhinovirus, and the like. In some embodiments, the viral infection is an infection of a virus selected from HIV, HBV, dengue virus, or Zika virus. In some embodiments, the viral infection is an infection of a coronavirus (e.g., SARS, MERS, etc). In some embodiments, the viral infection is a SARS-CoV-2 infection, including an infection of any strain or variant of SARS-CoV-2. Thus, in some embodiments, the pathogen infection is a SARS-CoV-2 infection and the subject in need of treatment has COVID-19.

In some embodiments, the administering is performed intranasally. In some embodiments, the administering is performed via oral, intravenous, subcutaneous, intramuscular, or ocular administration.

In some embodiments, the subject is a mammal. In some embodiments, the subject is a human. In some embodiments, the subject is a subject who has been diagnosed with a pathogen (e.g., viral or bacterial) infection. In some embodiments, the subject is a subject who has been exposed to others diagnosed with a pathogen (e.g., viral) infection and/or who has been exposed to others suspected of having a pathogen (e.g., viral) infection. In some embodiments, the subject is a subject who has traveled to an area with a high rate of infection of the pathogen. In some embodiments, the pathogen infection is a respiratory viral infection. In some embodiments, the respiratory viral infection is a coronavirus infection. In some embodiments, the viral infection is a SARS-CoV-2 infection.

In some embodiments, the subject is a subject who has a SARS-CoV-2 infection, is suspected of having a SARS-CoV-2 infection (e.g., by exhibiting one or more symptoms of a SARS-CoV-2 infection), or is a subject who has one or more increased risk factors for a SARS-CoV-2 infection. Increased risk factors for getting a SARS-CoV-2 infection include, but are not limited to, contact with another who has tested positive for SARS-CoV-2 infection or who has exhibited symptoms of SARS-CoV-2 infection, e.g., fever, chills, cough, difficulty breathing, headache, fatigue, sore throat, muscle or body ache, loss of smell or taste, nausea, and diarrhea; living in communal housing (e.g., a nursing home); and travel to areas with high rates of SARS-CoV-2 infectivity. In some embodiments, the subject is a subject who has increased risk for severe COVID-19 (e.g., risk for disease requiring hospitalization, ventilation or oxygen support, and/or that results in death). Subjects with increased risk of severe disease include human subjects over the age of 45 or over the age of 65. Underlying medical conditions or comorbidities that can result in higher risk of severe COVID-19 include, but are not limited to, cancer, chronic kidney disease, chronic lung disease (e.g., chronic obstructive pulmonary disease (COPD), asthma, cystic fibrosis, pulmonary hypertension, and interstitial lung disease), dementia, diabetes, Down's syndrome, heart disease (e.g., heart failure, coronary artery disease, hypertension, etc.), IV, a compromised or weakened immune system, liver disease (e.g., cirrhosis), being overweight (i.e., having a body mass index (BMI)>25 kg/m²) or obese (i.e., having a BMI³ 30 kg/m²), pregnancy, sickle cell disease or thalassemia, a history of smoking, stroke, and having a history of substance abuse. In some embodiments, the presently disclosed composition can be used prophylactically, e.g., by administration to a subject (e.g., a subject at higher risk of severe COVID-19)) planning or expecting to be in a crowded/unventilated area, a medical facility, or traveling to an area with a high rate of infectivity.

### III. Formulations

As noted above, the compositions of the presently disclosed subject matter comprise, in some embodiments, a composition that includes a pharmaceutically acceptable carrier. Any suitable pharmaceutical formulation can be used to prepare the compositions for administration to a subject. In some embodiments, the composition and/or carriers can be pharmaceutically acceptable in humans.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the subject; and aqueous and non-aqueous sterile suspensions that can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are sodium dodecyl sulfate (SDS), in one example in the range of 0.1 to 10 mg/ml, in another example about 2.0 mg/ml; and/or mannitol or another sugar, for example in the range of 10 to 100 mg/ml, in another example about 30 mg/ml; and/or phosphate-buffered saline (PBS).

It should be understood that in addition to the ingredients particularly mentioned above, the formulations of this presently disclosed subject matter can include other agents conventional in the art having regard to the type of formulation in question. For example, sterile pyrogen-free aqueous and non-aqueous solutions can be used.

### IV. Subjects

The methods and compositions disclosed herein can be used on a sample either in vitro (for example, on isolated cells or tissues) or in vivo in a subject (i.e., living organism, such as a patient). In some embodiments, the subject or patient is a human subject, although it is to be understood that the principles of the presently disclosed subject matter indicate that the presently disclosed subject matter is effective with respect to all vertebrate species, including mammals, which are intended to be included in the terms “subject” and “patient”. Moreover, a mammal is understood to include any mammalian species for which employing the compositions and methods disclosed herein is desirable, particularly agricultural and domestic mammalian species.

As such, the methods of the presently disclosed subject matter are particularly useful in warm-blooded vertebrates. Thus, the presently disclosed subject matter concerns mammals and birds. More particularly provided are methods and compositions for mammals such as humans, as well as those mammals of importance due to being endangered (such as Siberian tigers), of economic importance (animals raised on farms for consumption by humans), and/or of social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos or as pets (e.g., parrots), as well as fowl, and more particularly domesticated fowl, for example, poultry, such as turkeys, chickens, ducks, geese, guinea fowl, and the like, as they are also of economic importance to humans. Thus, also provided is the treatment of livestock including, but not limited to domesticated swine (pigs and hogs), ruminants, horses, poultry, and the like.

### V. Administration

Suitable methods for administration of a composition of the presently disclosed subject matter include, but are not limited to intravenous injection, intramuscular injection, oral administration, subcutaneous administration, intraperitoneal injection, intracranial injection, rectal administration, ocular administration, and intranasal administration. In some embodiments, the administration is systemic. In some embodiments, a composition can be deposited at a site in need of treatment in any other manner, for example by spraying a composition within the pulmonary pathways. The particular mode of administering a composition of the presently disclosed subject matter depends on various factors, including the distribution and abundance of cells to be treated.

For delivery of compositions to pulmonary pathways, compositions of the presently disclosed subject matter can be formulated as an aerosol or coarse spray. Methods for preparation and administration of aerosol or spray formulations can be found, for example, in U.S. Pat. Nos. 5,858,784; 6,013,638; 6,022,737; and 6,136,295.

### VI. Doses

An effective dose of a composition of the presently disclosed subject matter is administered to a subject. An “effective amount” is an amount of the composition sufficient to produce detectable treatment. Actual dosage levels of constituents of the compositions of the presently disclosed subject matter can be varied so as to administer an amount of the composition that is effective to achieve the desired effect for a particular subject and/or target. The selected dosage level can depend upon the activity of the composition and the route of administration. In some embodiments, the dosage can be about 1 mg/kg to about 100 mg/kg.

After review of the disclosure herein of the presently disclosed subject matter, one of ordinary skill in the art can tailor the dosages to an individual subject, taking into account the particular formulation, method of administration to be used with the composition, and nature of the target to be treated. Such adjustments or variations, as well as evaluation of when and how to make such adjustments or variations, are well known to those of ordinary skill in the art.

### V. Antibodies, Peptides and Proteins

Antibodies directed against proteins, polypeptides, or peptide fragments thereof for use according to the presently disclosed subject matter can be generated using methods that are well known in the art. For instance, U.S. Pat. No. 5,436,157, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants can be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

In some embodiments, antibodies directed against pathogen (e.g., coronavirus) proteins can be of use in preparing the functionalized nanoparticles of the presently disclosed subject matter. Fragments of pathogen proteins (e.g., SARS-CoV-2 S protein) can be generated and antibodies prepared against the fragments. For the preparation of monoclonal antibodies, any technique which provides for the production of antibody molecules by continuous cell lines in culture can be utilized. For example, the hybridoma technique originally developed by Kohler and Milstein (1975, Nature 256:495-497), the trioma technique, the human B-cell hybridoma technique (Kozbor et al., 1983, Immunology Today 4:72), and the EBV-hybridoma technique (Cole et al., 1985, in Monoclonal Antibodies and Cancer Therapy, Alan R. Liss, Inc., pp. 77-96) can be employed to produce human monoclonal antibodies. In some embodiments, monoclonal antibodies are produced in germ-free animals.

In some embodiments, human antibodies can be used and obtained by utilizing human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, Inc., pp. 77-96). Furthermore, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for epitopes of viral polypeptides together with genes from a human antibody molecule of appropriate biological activity can be employed; such antibodies are within the scope of the presently disclosed subject matter. Once specific monoclonal antibodies have been developed, the preparation of mutants and variants thereof by conventional techniques is also available.

In some embodiments, techniques described for the production of single-chain antibodies (U.S. Pat. No. 4,946,778, incorporated by reference herein in its entirety) are adapted to produce protein-specific single-chain antibodies. In another embodiment, the techniques described for the construction of Fab expression libraries (Huse et al., 1989, Science 246:1275-1281) are utilized to allow rapid and easy identification of monoclonal Fab fragments possessing the desired specificity for specific antigens, proteins, derivatives, or analogs of the presently disclosed subject matter.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

The generation of polyclonal antibodies is accomplished by inoculating the desired animal with the antigen and isolating antibodies which specifically bind the antigen therefrom.

Monoclonal antibodies directed against full length or peptide fragments of a protein or peptide can be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide can also be synthesized using chemical synthesis technology. Alternatively, DNA encoding the desired peptide can be cloned and expressed from an appropriate promoter sequence in cells suitable for the generation of large quantities of peptide. Monoclonal antibodies directed against the peptide are generated from mice immunized with the peptide using standard procedures as referenced herein.

A nucleic acid encoding the monoclonal antibody obtained using the procedures described herein can be cloned and sequenced using technology which is available in the art, and is described, for example, in Wright et al. (1992, Critical Rev. in Immunol. 12(3,4):125-168) and the references cited therein. Further, the antibody of the presently disclosed subject matter can be “humanized” using the technology described in Wright et al., (supra) and in the references cited therein, and in Gu et al. (1997, Thrombosis and Hematocyst 77(4):755-759).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage that encode the desired antibody can be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art.

Processes such as those described above have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures presented above describe the generation of phage which encode the Fab portion of an antibody molecule. However, the presently disclosed subject matter should not be construed to be limited solely to the generation of phage encoding Fab antibodies. Rather, phage which encode single chain antibodies (scFv/phage antibody libraries) are also included in the presently disclosed subject matter. Fab molecules comprise the entire Ig light chain, that is, they comprise both the variable and constant region of the light chain but include only the variable region and first constant region domain (CHI) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA can be generated following the procedures described in Marks et al., 1991, J. Mol. Biol. 222:581-597. Panning of phage so generated for the isolation of a desired antibody is conducted in a manner similar to that described for phage libraries comprising Fab DNA.

The presently disclosed subject matter should also be construed to include synthetic phage display libraries in which the heavy and light chain variable regions can be synthesized such that they include nearly all possible specificities (Barbas, 1995, Nature Medicine 1:837-839; de Kruif et al. 1995, J. Mol. Biol. 248:97-105).

In the production of antibodies, screening for the desired antibody can be accomplished by techniques known in the art, e.g., ELISA (enzyme-linked immunosorbent assay). Antibodies generated in accordance with the presently disclosed subject matter can include, but are not limited to, polyclonal, monoclonal, chimeric (i.e., “humanized”), and single chain (recombinant) antibodies, Fab fragments, and fragments produced by a Fab expression library.

The peptides and proteins of the presently disclosed subject matter (e.g., ACE2 proteins) can be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions that will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method that utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

To ensure that the proteins or peptides obtained from either chemical or biological synthetic techniques is the desired peptide, analysis of the peptide composition should be conducted. Such amino acid composition analysis can be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, can also be used to determine definitely the sequence of the peptide.

Prior to its use, the peptide can be purified to remove contaminants. In this regard, it will be appreciated that the peptide will be purified to meet the standards set out by the appropriate regulatory agencies. Any one of a number of a conventional purification procedures can be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure peptide obtained as described herein can be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Chemicals and Reagents

Polylactic acid (PLA), polyvinyl alcohol (PVA), cholesterol, 1-bromoheptadeca fluorooctane (perfluorooctylbromide or PFOB), dichloromethane, 3,3′-dioctadecyloxacarbocyanine perchlorate (DiO), dimethyl sulfoxide (anhydrous) and phorbol 12-myristate 13-acetate (PMA) were purchased from Sigma-Aldrich (St. Louis, Missouri, United States of America). 1,2-distearoyl-sn-glycero-3-phosphocholine (DSPC), 1,2-dioleoyl-sn-glycero-3-phospho-L-serine (sodium salt) (DOPS), 1,2-distearoyl-sn-glycero-3-phosphoethanolamine-N-[methoxy(polyethylene glycol)-2000](DSPE-mPEG₂₀₀₀), 1,2-distearoyl-sn-glycero-3-phosphoethanolamine-N-[biotinyl (polyethylene glycol)-2000](ammonium salt) (DSPE-PEG₂₀₀₀-biotin), 1,2-distearoyl-sn-glycero-3-phosphoethanolamine-N-[carboxy(polyethylene glycol)-2000, NHS ester](DSPE-PEG₂₀₀₀-NHS) were purchased from Avanti Polar Lipids (Alabaster, Alabama, United States of America). A luciferase substrate sold under the tradename RENILLA-GLO™ was purchased from Promega (Madison, Wisconsin, United States of America). 1,1-dioctadecyl-3,3,3,3-tetramethylindodicarbocyanine (DiD), wheat germ agglutinin CF™488A conjugate and wheat germ agglutinin Cf532 conjugates were ordered from Biotium (Fremont, California, United States of America). Zombie NIR™ Fixable Viability Kit was ordered from BioLegend (San Diego, California, United States of America) Cell Counting Kit-8 (CCK8) was purchased from MedChem Express (Monmouth Junction, New Jersey, United States of America). 384-well tissue culture plates were purchased from Santa Cruz Biotechnology (Dallas, Texas, United States of America). Biotinylated recombinant human ACE2 was purchased from Bioss Antibodies (Woburn, Massachusetts, United States of America). ACE2 (E-11) Alexa Fluor 488 was purchased from Santa Cruz Biotechnology (Dallas, Texas, United States of America; cat #sc-390851 AF488, RRID: AB_2861379). Mouse anti-SARS-CoV-2 (2019-nCoV) Spike Neutralizing Antibody was purchased from SinoBiological (Beijing, China; cat #40592-MM57, RRID: AB_2857935). SARS-CoV-2 spike pseudotyped lentivirus with luciferase reporter gene was purchased from Integral Molecular (Philadelphia, Pennsylvania, United States of America; Catalog #RVP 701). Endosome antibody anti-EEA1-AF594 was purchased from abcam (Cambridge, United Kingdom; Catalog #ab206913, clone: EPR4245). Lysosome antibody anti-LAMP-1-AF488 was purchased from BioLegend (San Diego, California, United States of America; Catalog #328609, clone: H4A3, RRID: AB_1227505). Phycoerythrin (PE) beads sold under the tradename QUANTIBRITE™ were purchased from BD Biosciences (San Jose, Califomia, United States of America). IL-6 ELISA was obtained from Biolegend (San Diego, California, United States of America). A centrifugal filter unit sold under the tradename AMICON® Ultra-15 with Ultracel-100 membrane (100 kDa, 4 mL) (Merck KGaA, Darmstadt, Germany) was purchased from EMD Millipore (Burlington, Massachusetts, United States of America). 32% paraformaldehyde aqueous solution was purchased from Electron Microscopy Sciences (Hatfield, Pennsylvania, United States of America).

### Cell Lines

HEK293T-ACE2 cells were provided by Integral Molecular (Philadelphia, Pennsylvania, United States of America; Integral Cat #C-HA102) and were cultured by cell culture media containing DMEM+10% FBS+10 mM HEPES+1% Penicillin-Streptomycin. Human lung epithelial cell line A549 cells (sold under the tradename CCL-185™, American Type Culture Collection (ATCC), Manassas, Virginia, United States of America) were cultured in DMEM+10% FBS+1% Penicillin-Streptomycin. THP-1 cells were kindly provided by Dr. Aaron Esser-Kahn and cultured in RPMI 1640+10% FBS+1% Penicillin-Streptomycin. All cells were maintained in a CO₂ incubator (sold under the tradename STERI-CYCLE® i250, Thermo Fisher Scientific, Waltham, Massachusetts, United States of America) at 37° C. and 5% CO₂. Vero E6 cells (American Type Culture Collection (ATCC), Manassas, Virginia, United States of America) were infected under biosafety level 3 conditions with SARS-CoV-2 (nCoV/Washington/1/2020, kindly provided by the National Biocontainment Laboratory, Galveston, Texas, United States of America).

### Example 1

### Synthesis of Functionalized Nanoparticles

To synthesize the functionalized nanoparticles, a two-step method was developed for polymer-lipid hybrid nanoparticles, in which the polymer and lipid components were prepared separately and combined at the end of the process. The PLA nanoparticles were prepared in accordance with existing methods⁵⁶ through an oil-in-water emulsion solvent evaporation process. 100 mg of PLA and 100 μL of perfluorooctylbromide (PFOB) were dissolved in 3.5 mL of dichloromethane. The organic phase was mixed with 20 mL of 2.0% PVA solution. The mixture was emulsified by sonication on ice for 2 minutes. The dichloromethane in the emulsified mixture was evaporated under magnetic stirring at 300 rpm for 3-4 hours at room temperature. The resulting solution was centrifuged and the pellets were washed with PBS 3 times (5,000×g for 10 minutes), and then the pellets were lyophilized and stored at 4° C. before use. For any fluorescence labeling, 0.05 mg of a lipophilic carbocyanine dye, DiD or DiO, was added into the 100 mg PLA organic phase when preparing the nanoparticle core. For PLA nanoparticles with size 200 nm, 100 mg of PLA was emulsified with 2.0% PVA and the supernatant was collected after centrifugation. For PLA nanoparticles with size 500 nm, 100 mg of PLA and 100 μL PFOB were mixed, then emulsified with 2.0% PVA and the pellets were collected after centrifugation. For PLA nanoparticles with size 1200 nm, 100 mg of PLA and 100 μL PFOB were mixed and emulsified with 1.0% PVA and the pellets were collected after centrifugation.

Functionalized PLA-nanoparticles composed of 15% phosphatidylserine and 0.5% DSPE-PEG₂₀₀₀-biotin were prepared by dissolving DOPS (0.388 μmol), DSPC (1.292 μmol), cholesterol (0.775 μmol), DSPE-mPEG₂₀₀₀ (0.166 μmol) and DSPE-PEG₂₀₀₀-biotin (0.013 μmol) at a molar ratio of 10:120:60:9:1 in dichloromethane. The dichloromethane solvent was slowly evaporated by heating the lipid solution at 55° C. to remove the solvent and further dried in a vacuum drying oven to produce a dried lipid film. The lipid film was reconstituted in 2 mL of PBS (pH 7.4) containing 0.2 mg PLA nanoparticles and the contents were hydrated at 60° C. under ultrasonication (Branson CPX5800H). Then the mixture was sonicated with a sonicator probe (Fisher Scientific, Hampton, New Hampshire, United States of America) for 2 minutes (100 W, 22.5 kHz, 30% amplitude). Formulations for functionalized nanoparticles with different phosphatidylserine surface densities were listed in Table 1.

TABLE 1
Formulations of lipid shell with different phosphatidylserine
densities (molar ratios).

| | | | Choles- | DSPE- | DSPE- |
|---|---|---|---|---|---|
| Formulation | DOPS | DSPC | terol | mPEG₂₀₀₀ | PEG₂₀₀₀-biotin |
| 0% PS | 0% | 65% | 30% | 4.5% | 0.5% |
| 5% PS | 5% | 60% | 30% | 4.5% | 0.5% |
| 10% PS | 10% | 55% | 30% | 4.5% | 0.5% |
| 15% PS | 15% | 50% | 30% | 4.5% | 0.5% |

To synthesize Nanotrap-ACE2, 1 mg of the biotin functionalized PLA nanoparticles were then incubated with streptavidin (34.25 μL, 2 g/L) on ice for 40 minutes under magnetic stirring. The resulting solution was centrifuged at 5,000×g for 10 minutes and washed with 1 mL PBS for 3 times to remove excess streptavidin. The pellet was resuspended in 100 μL PBS and incubated with biotinylated ACE2 (Bioss Antibodies, Woburn, Massachusetts, United States of America) for 30 minutes on ice. The resulting PLA®DOPS/biotin˜SA˜ACE2 nanoparticle was termed as Nanotrap-ACE2.

Nanotrap-Antibody was synthesized by combining 15% DOPS (0.313 mg, 0.386 μmol), 50% DSPC (1.018 mg, 1.287 μmol), 30% cholesterol (0.300 mg, 0.773 mol) and 5% DSPE-PEG₂₀₀₀-NHS (0.370 mg, 0.129 μmol) in dichloromethane. The lipids mixtures were vacuum dried overnight, and the resulting thin film was hydrated with PLA-NPs PBS solution under ultrasonic water bath and further reacted with 100 g of SARS-COV-2 neutralizing antibody for 4 hours at 4° C.

### Example 2

### Characterization of Functionalized Nanoparticles

The sizes of functionalized nanoparticles were measured by a dynamic light scattering particle size analyzer (Malvern ZETASIZER®, Malvern Instruments Limited, Malvern, United Kingdom). Briefly, 1 μL of functionalized nanoparticles were dispersed in 0.1×PBS and further dispersed in ultrasonic water bath for 10 minutes before testing. The size measurement was carried at 25° C. with count rates within 300-500 kcps and measured 3 times. The zeta potentials of functionalized nanoparticles were performed by a Möbiuζ system (Wyatt Technology, Santa Barbara, California, United States of America). The data were presented as mean±SD.

AF488-labeled anti-ACE2 antibody (Santa Cruz Biotechnology, Inc., Dallas, Texas, United States of America) was added to the Nanotrap-ACE2 for 30 minutes on ice and centrifuged at 5,000×g for 10 min, the pellet was washed with PBS for 3 times. The resulting functionalized nanoparticles were resuspended in 50% glycol and imaged by total internal reflection fluorescence microscopy (Nikon) with 488 nm and 647 nm excitation lasers and 200 milliseconds exposure. Line scans were performed in Fiji.

The sizes and morphologies of the functionalized nanoparticles were studied by scanning electron microscopy (SEM). Briefly, 10 μL of exemplary functionalized nanoparticle Nanotrap-ACE2 was diluted in MiliQ water and further dispersed on ultrasonic water bath for 10 minutes before adding onto a silicon chip. 40 L of SARS-CoV-2 spike pseudotyped lentivirus was fixed by 4% PFA at 37° C. for 30 minutes, the virus was then washed by PBS 3 times using a centrifugal filter sold under the tradename AMICON® Ultra-15 (Merck KGaA, Darmstadt, Germany; pore size 100 KDa) at 3,000×g, 10 minutes. The resulting fixed virus was incubated with 10 μL Nanotraps-ACE2 at 37° C. for 1 hour and added onto the 1 cm² silicon chip followed by airdrying overnight. After dehydration, the samples were coated with 8 nm platinum/palladium by sputter coater (Cressington 208HR, Cressington Scientific Instruments, Watford, United Kingdom). The scanning electronic microscope sold under the tradename MERLIN™ (Carl Zeiss AG, Jena, Germany) was used to image the morphology of the functionalized nanoparticles with an accelerating voltage of 2.0 kV. For each sample, more than 10 measurements with different magnification were performed to ensure the repeatability of the results. For transmission electron microscopy (TEM) imaging, 10 μl of functionalized nanoparticle solution was drop-cast on a carbon film supported TEM grid (Ted Pella Inc, Redding, California, United States of America, product number 01843) which was pre-treated with oxygen plasma. Then the grid was gently rinsed with de-ionized (DI) water droplets and stained with 1% uranyl acetate aqueous solution for 1 min. The resultant solution was gently removed with filter paper. The sample was then imaged by a FEI Tecnai G2 F30 electronic microscope (FEI Company, Hillsboro, Oregon, United States of America) at 300 kV after thoroughly drying.

For long-term storage, the functionalized nanoparticles were freeze-dried into solid powder using a lyophilizer (Freezone 6, Labconco, Kansas City, Missouri, United States of America). Briefly, the functionalized nanoparticle solution was transferred into Eppendorf tubes and frozen on dry ice. The lid of the frozen tube was removed and quickly embedded with parafilm; holes were punctured in the parafilm using pipette tips. The tubes were placed in the glass tank connected to a lyophilizer with lids up. The lyophilizer was vacuumed using an oil-pump such that any water in the tube was sublimated under −53.3° C. for 24 h, resulting in the functionalized nanoparticle powder. The tubes with functionalized nanoparticle powder were sealed by parafilm and immediately stored in a −20° C. freezer. The functionalized nanoparticle powder was reconstructed six months later by adding PBS solution followed by ultrasonication.

The density of surface ACE2 or neutralizing antibody on the functionalized nanoparticle surface was measured by flow cytometry⁶². DiD-loaded Nanotrap-ACE2 was stained with anti-ACE2-PE (Sino Biological, Beijing, China) for 30 min on ice. DiD-loaded Nanotrap-Antibody was stained with anti-IgG-PE (BioLegend, San Diego, California, United States of America) for 30 min on ice. Double positive populations were gated, and then phycoerythrin (PE) beads (sold under the tradename QUANTIBRITE™, BD Biosciences, San Jose, California, United States of America) were used to calculate surface densities according to manufacturer's instructions.

### Example 3

### Flow Cytometry Analysis of Functionalized Nanoparticle Phagocytosis

Macrophage differentiation was conducted as follows⁵¹: THP-1 cells were treated with 150 nM PMA for 24 hours and replaced by fresh culture medium for another 24 hours. The differentiated THP-1 cells were harvested as dTHP-1 macrophages and maintained in RPMI supplemented with 10% FBS and 1% Penicillin-Streptomycin. The dTHP-1 macrophages were then released from the plate with Trypsin-EDTA (0.25%) and cell number was counted by a hemocytometer. 1 million dTHP-1 macrophages were seeded into each 6-well plate overnight, and DiO-labeled functionalized nanoparticles (200, 500, and 1200 nm) were incubated with dTHP-1 macrophages for 0, 24, or 48 hours. For phosphatidylserine investigation, dTHP-1 macrophages were incubated with functionalized nanoparticles containing different phosphatidylserine molar ratios (0%, 5%, 10%, 15%) for 0, 2, 4, 6, 24, or 48 hours. The cells were harvested and washed 3 times with PBS at 300×g for 5 minutes, and stained with dyes from a kit sold under the tradename Zombie NIR™ Fixable Viability Kit (BioLegend, San Diego, California, United States of America) on ice for 10 minutes. Then cells were washed with FACS buffer (PBS, 10% FBS, 0.1% NaN₃) 2 times and resuspended in 200 μL FACS buffer. Flow cytometry was carried on a flow cytometer sold under the tradename BD LSRFORTESSA™ (Becton Dickinson and Company, Franklin Lakes, New Jersey, United States of America). Live and single cells were gated and DiO fluorescence channel was used to indicate the phagocytosis efficiency of different functionalized nanoparticles. The data were further analyzed by data analysis software sold under the tradename FLOWJO® (FlowJo LLC, Ashland, Oregon, United States of America) and Prism software (Graphpad Software Inc., La Jolla, California, United States of America).

### Example 4

### Lattice Light-Sheet Microcopy Imaging Analysis of Functionalized Nanotrap Phagocytosis

For lattice light-sheet imaging, 4×10⁴ dTHP-1 macrophages were seeded onto each coverslip and DiD-labeled functionalized nanoparticles with different phosphatidylserine molar ratios (0%, 10%, and 15%) were added for 24- or 48-hour incubation. The cells were washed by PBS for 3 times, fixed with 4% PFA, stained with (5 g/mL) CF488 Wheat Germ Agglutinin (WGA) Conjugates (Biotium, Fremont, California, United States of America), and washed 3 times with HBSS. Coverslips were imaged by lattice light-sheet microscopy (3i) using z+objective scanning. Imaging was conducted with 488 nm and 647 nm lasers, with dither set to 3 and 20 millisecond exposures. Z-steps (60) were collected with a 0.4 m step size. Resulting images were deconvolved as described previously^{58,59} with LLSpy (cudaDeconv) used under license from Howard Hughes Medical Institute, Janelia Research Campus. Image reconstruction videos were made in Imaris (Bitplane, Belfast, United Kingdom).

### Example 5

### Confocal Microscopy of Endosomes and Lysosomes

For confocal imaging, 3×10⁴ dTHP-1 cells were seeded into 8-well chambers and 500 nm 15% PS DiD-labeled functionalized nanoparticles were added for 6 h at 37° C. The cells were washed by PBS for 3 times, fixed with 4% PFA, and washed 3 times with PBS. The cells were then stained with (10 μg/mL) anti-LAMP-1-AF488 (BioLegend, San Diego, California, United States of America) and (0.5 g/mL) anti-EEA1-AF594 (Abcam, Cambridge, United Kingdom) for 30 min, then washed 3 times with PBS. 300 nM DAPI was then added for 10 min, then washed 3 times with PBS. Confocal imaging was conducted on a Leica SP8 with a white light laser and 100× oil-immersion objective with 1.5× zoom. Z-stacks were acquired with a z-step of 300 nm. Images were z-projected and despeckled in Fiji. Line scan plot profile was conducted in Fiji.

### Example 6

### Kinetics Assays

SARS-CoV-2 spike pseudotyped lentivirus was labeled with lipophilic dye DiO⁶³ for 20 min, fixed with 4% PFA then washed 3 times with PBS in an centrifugal tube sole under the tradename AMICON® Ultra-centrifugal tube (100 kDa; Merck KGaA, Darmstadt, Germany). DiD-loaded functionalized nanoparticles were prepared and incubated with the DiO labeled pseudotyped lentivirus for various time points. DiD/DiO double positive events were gated and the mean fluorescence intensity of DiO was recorded by flow cytometry until saturation was achieved.

Macrophages were prepared as described above and incubated with DiO-loaded functionalized nanoparticles for varying time points until saturation was achieved. The cells were harvested and washed 3 times with PBS at 300×g for 5 min, and stained using a viability kit sold under the tradename Zombie NIR™ Fixable Viability Kit (BioLegend, San Diego, California, United States of America) on ice for 10 min. Then cells were washed with FACS buffer (PBS, 10% FBS, 0.1% NaN₃) 2 times and resuspended in 200 μL FACS buffer. Flow cytometry was carried on a flow cytometer sold under the tradename LSRFORTESSA™ (Becton Dickinson and Company, Franklin Lakes, New Jersey, United States of America). Live and single cells were gated and DiO fluorescence channel was used to indicate the phagocytosis efficiency at various time points.

### Example 7

### Enzyme-Linked Immunosorbent Assay (ELISA)

For interleukin-6 (IL-6) ELISA, six conditions were conducted to test macrophage activation: 1) Macrophages alone 2) Macrophages and functionalized nanoparticles (Nanotraps) 3) Macrophages and Epithelial cells 4) Macrophages, Epithelial cells, and Virus 5) Macrophages, Epithelial Cells, and functionalized nanoparticles (Nanotraps) and 6) Macrophages, Epithelial Cells, Virus, and functionalized nanoparticles (Nanotraps). For all wells, 1×10⁵ dTHP-1 cells were plated into 12-well plates overnight. The next day, to wells containing epithelial cells, 2×10⁵ HEK293-ACE2 cells were added. To wells that include functionalized nanoparticles, 10 g/mL functionalized nanoparticles were added. To wells that include functionalized nanoparticles and virus, 10 g/mL functionalized nanoparticles were added to 500 FFU pseudotyped VSV, incubated for 1 h at 37° C., then added to the cells. Plates were incubated for 24 h, then centrifuged at 300×g for 5 min and supernatants were harvested. IL-6 ELISA (BioLegend, San Diego, California, United States of America) was conducted according to manufacturer's instructions.

### Example 8

### Production of SARS-CoV-2 Spike Pseudotyped VSV

Packaging cells (HEK293T) in serum-free DMEM were transfected with 9 g pCAGGS SARS-CoV-2 spike expression plasmid using polyethylenimine (PEI). After 24 hours, 3×10⁷ FFU VSVdG*G-GFP virus was added to the HEK293T cells and incubated for another 48 hours. Media were collected and spun at 500×g for 3 minutes to remove cell debris, then passed through a 0.45 m pore filter. The virus was then stored at −80° C. To check the infection rate, HEK293T-ACE2 cells (Integral Cat #C-HA102, Integral Molecular, Inc., Philadelphia, Pennsylvania, United States of America) were incubated with the final VSVdG-GFP*CoV2 pseudovirus and visualized by the presence of GFP positive cells through direct microscopic imaging or flow cytometry.

### Example 9

### SARS-CoV-2 Pseudovirus Neutralizing Assay

For SARS-CoV-2 spike pseudotyped VSV neutralizing assay, 4×10⁴ HEK293T-ACE2 cells (maintained in DMEM supplemented with 10% FBS and 1% Penicillin-Streptomycin) were seeded in a 96-well plate overnight. Different concentrations of ACE2 proteins or functionalized nanoparticle (containing 0.1, 0.2, 0.4, 0.6, 0.8, 1.0, 1.5, 3.0, 6.0, 9.0, 12, 15 μg/mL ACE2) were incubated with 500 FFU SARS-CoV-2 spike pseudotyped VSV for 1 hour in 37° C. The virus-Nanotrap solution was added into the HEK293T-ACE2 cells and incubated for 24 hours (n=3 for each group). The cells were imaged with a fluorescence microscope (Nikon) using 10×/0.30 NA objective. The excitation wavelengths were 470±25 nm (Spectra X, Lumencor, Beaverton, Oregon, United States of America). The emissions of GFP were captured by an Andor iXon Ultra 888 back-illuminated EMCCD camera (Oxford Instruments Plc, Abingdon, United Kingdom). The number of GFP-positive cells were counted manually by objective three times. The viral infection rates were calculated as the ratio of GFP-positive cells in the group incubated to that of the group incubated with virus alone.

For SARS-CoV-2 spike pseudotyped lentivirus neutralizing assay, 1×10⁴ HEK293T-ACE2 cells were seeded onto a 384-well plate overnight. Functionalized nanoparticles or ACE2 was added to SARS-CoV-2 pseudovirus (4 μL per well) and incubated for 1 hour at 37° C. The virus-functionalized nanoparticle solution was added to each well (n=3 for each group). 72 hours later, the plate was centrifuged for 5 minutes at 500×g to prevent cell loss. Supernatant was aspirated and 35 μL of PBS was added. PBS was carefully aspirated, leaving ˜15 μL of liquid behind. sold An assay substrate sold under the tradename RENILLA-GLO™ (Promega, Madison, Wisconsin, United States of America) was added to the Assay Buffer at a 1:100 dilution, then 15 μL of the substrate:buffer was added to each well of a 384-well plate. The bioluminescence was recorded by a microplate reader sold under the tradename BIOTEK CYTATION™ 5 (BTI Holdings, Winooski, Vermont, United States of America) with an exposure of 200 milliseconds. Wells infected with pseudovirus only were normalized as 100%.

### Example 10

### Co-Culture Assay

THP-1 cells were differentiated into macrophages as described above. Coculture was carried out in a macrophage to A549 cell ratio of 1:5. 4×10⁴ A549 cells were seeded in an 18-well microslide (Vivid) overnight and 8×10³ dTHP-1 macrophages were added onto the A549 cells for another 6 hours. 500 FFU SARS-CoV-2 spike pseudotyped VSV was incubated with functionalized nanoparticles or PBS in 37° C. for 1 hour before adding to the coculture cells. 24 hours later, the cells were fixed with 4% PFA and stained with CF532 Wheat Germ Agglutinin (WGA) Conjugates (Biotium, Fremont, California, United States of America), and DAPI and imaged under a confocal microscope (Leica SP8, Wetzlar, Germany). Percent infectivity was quantified in FIJI by dividing GFP⁺ cells by total cell number (DAPI-stained nuclei). Each channel was processed as follows: Image>Threshold (“Huang” preset⁶⁰); Image>Binary>Fill Holes; Image>Binary>Watershed; Analyze>Count Particles.

### Example 11

### In Vitro Cytotoxicity Assay

A549 or HEK293T-ACE2 cells (both maintained in DMEM supplemented with 10% FBS and 1% Penicillin-Streptomycin) were seed in a 96-well plate at a density of 1×10⁴ cells/well in 100 μL of culture medium overnight. Functionalized nanoparticles (3.8×10⁷ particles/mL) were added into cells and the cells were cultured in a CO₂ incubator at 37° C. for 72 hours. 10 μL of CCK-8 (MedChem Express, Princeton, New Jersey, United States of America) solution was added to each well of the plate. The plate was incubated for 2 hours in the incubator. Then it was put into a microplate reader sold under the tradename BIOTEK CYTATION™ 5 (BTI Holdings, Winooski, Vermont, United States of America) and the plate was gently shaken for 1 minute before measuring the absorbance at 450 nm. The cytotoxicity was calculated by cell viability, that the relative absorbance from the control wells without functionalized nanoparticles were normalized as 100%. The functionalized nanoparticle concentrations (particles/mL) were manually counted by a hemocytometer. Briefly, the freshly prepared functionalized nanoparticles were vortexed and diluted in PBS in 1 to 1000 dilution. 10 μl of the diluted solution was further mixed with 10 μl Trypan blue for enhanced contrast, and added onto the hemocytometer, then the number of the functionalized nanoparticles were counted under a microscope.

### Example 12

### In Vivo Biosafety Assays

C57BL/6NHsd mice at the age of 6 weeks were purchased from Envigo (Indianapolis, Indiana, United States of America). To evaluate the safety of the functionalized nanoparticles, 2 male and 2 female 6-10-week-old C57BL/6NHsd mice were intratracheally administered with 10 mg/kg Nanotrap-ACE2 in 50 μL PBS. Blood samples were collected by submandibular vein via cheek punch using a commercially available 4 mm point lancet after three days. A small aliquot of approximately 100 L blood was collected into EDTA-containing heparinized tubes, and red blood cells, white blood cells, and platelets were counted by an Act Diff 5 CP Hematology Analyzer (Beckman Coulter, Brea, California, United States of America) according to the manufacturer's instructions. For the comprehensive chemistry panels, blood was allowed to coagulate in 4° C. for 2 hours, and serum was collected after centrifugation (1,000×g for 15 minutes) for analysis. Serum alkaline phosphatase, alanine aminotransferase, amylase, urea nitrogen, calcium, cholesterol, glucose, total bilirubin, total proteins were determined by a Vet Axcel blood chemistry analyzer (Alfa Wasserman, West Caldwell, New Jersey, United States of America). Lungs, heart, liver, spleen, and kidney were collected from the same mice, fixed in 10% formalin for 24 hours, embedded in paraffin. The resulting blocks were cut in 5 μm sections and further stained with hematoxylin and eosin (H&E) by the University of Chicago Human Tissue Research Center. Fluorescent imaging samples were collected in a specimen matrix sold under the tradename TISSUE-TEK® O.C.T. Compound (Sakura Finetek, Tokyo, Japan) on dried ice and stored in −80° C. before cryosectioning on a cryostat (Leica, Wetzlar, Germany). The obtained 10 m thick tissue slides were then further stained with DAPI. The histology and fluorescence slides were scanned by a 20× whole slide scanner sold under the tradename PANNORAMIC® MIDI (3D HISTECH, Ltd., Budapest, Hungary) and analyzed on the QuPath software.

### Example 13

### Ex Vivo Lung Perfusion Assay

Non-transplantable human lungs were obtained from deceased individuals provided by the organ procurement organization Gift of Hope (Itasca, Illinois, United States of America). All specimens and data were de-identified prior to receipt.

Lung Harvest. Living lungs unsuitable for transplantation were harvested in standard clinical fashion⁵⁷ from deceased patients. FIG. 5a shows the lung of a 56-year-old male patient (87.2 kg, cause of death: brain death). Lungs were transported to the laboratory at 4° C.

Lung inoculation. Tissue samples were collected from the edge of the right superior lobe before perfusion as a untreated control; 500 μL SARS-CoV-2 spike pseudotyped lentivirus (RVP 701, Integral Molecular, Philadelphia, Pennsylvania, United States of America) was resuspended in 5 mL PBS and injected into the lingula of the left lung; 500 L SARS-CoV-2 spike pseudotyped lentivirus was first incubated with Nanotrap-Antibody carrying 250 g neutralizing antibody for 1 hour at 37° C. before inoculation. This mixture was then injected into the right middle lobe. Tissue samples at all three sites were collected. Some samples were immersed in MACS buffer for tissue dissociation, and other samples were fixed in 4% PFA, sliced and stained for H&E.

Lung perfusion. The lung bloc was perfused according to published techniques^{25,61}. A centrifugal pump was used to perfuse the pulmonary artery with deoxygenated cellular perfusate (1×Dulbecco's Modified Eagle Medium containing 4.5 g/K D-glucose, L-glutamine and 110 mg/L of sodium pyruvate, with addition of 5% bovine serum albumin and 2 units of packed red blood cells). The left atrium was left open for gravity drainage. The trachea was incubated and the lung was ventilated with volume control ventilation (tidal volume set at 6-8 mL/weight of ideal body weight (kg) of donor, respiratory rate of 8-13, and fraction of inspired oxygen set at 21%). Sweep gas composed of 8% CO₂, 3% O₂, 89% N₂ was connected to the hollow-fiber de-oxygenator heat exchanger to remove oxygen and add in CO₂ into the perfusate returning back to the lung. After initiation of perfusion with gradual warming and increasing pump flow over 30 minutes, the lung bloc was maintained at 37° C. with pump flow calibrated to pulmonary artery pressure of 10-20 mmHg for 8 hours.

Sampling. Perfusate was sampled at serial time points from the pulmonary artery and left atrium. Differences in oxygen content between perfusate samples were used to calculate the oxygenation capacity of the lung. Airway pressure was measured periodically to calculate lung compliance based on tidal volume. Tissue samples were collected from treated and untreated lobes of the lung at time 0 and time 8 hours of perfusion.

Sample Processing. After 8 hours perfusion, tissues were harvested as described above. Tissue dissociation was performed by mechanical digestion in DMEM media treated with 2.5 U/mL DNase (company); samples were passed through 70 m cell strainers (Fisher Scientific, Hampton, New Hampshire, United States of America). Red blood cells were lysed in 10 mL RBC lysis buffer (Life Technologies, Carlsbad, California, United States of America), washed 3 times in DMEM (300×g for 3 minutes), and resuspended in 10 mL DMEM. Cells were counted with a hemocytometer and immediately used for luciferase assay or cryopreserved in Cell Banker medium (Amsbio, Abingdon, United Kingdom) at a density of 4×10⁶ cells/mL.

Samples that were not dissociated were fixed in 4% PFA and sliced with thickness of 5 μm. Some samples were stained for HandE and imaged on a CRi Pannoramic MIDI 20× whole slide scanner. Tissue slides used for RBC quantification were stained for DAPI and imaged on a 20× whole slide scanner sold under the tradename PANNORAMIC® MIDI (3D HISTECH, Ltd., Budapest, Hungary) with 488-nm and 560-nm lasers. Both channels were quantified in Fiji as follows: Image>Threshold (“Otsu” preset); Image>Binary>Fill Holes; Analyze>Measure.

For luciferase assay, the cells harvested as described above were washed with PBS (300×g for 3 minutes) 3 times. Then 2×10⁴ cells were seeded into 96-well plate. An assay substrate sold under the tradename RENILLA-GLO™ (Promega, Madison, Wisconsin, United States of America) was added to the Assay Buffer at a 1:100 dilution, then 30 μL of the Substrate Buffer was added to each well for 10 minutes. The bioluminescence from each well was detected by a microplate reader sold under the tradename BIOTEK CYTATION™ 5 (BTI Holdings, Winooski, Vermont, United States of America) with an exposure of 200 milliseconds.

Primary cells harvested from the untreated right superior lobe of the human lung were further used for infection analyses. Briefly, 1×10⁴ primary lung cells were seeded onto a 384-well plate. 20 μL Nanotraps-Antibody (3.8×10⁷ particles/mL) was added to 10 μL SARS-CoV-2 spike pseudotyped lentivirus (RVP 701, Integral Molecular, Philadelphia, Pennsylvania, United States of America) per well and incubated for 1 hour at 37° C. before adding to the cells. For virus only group, 10 μL SARS-CoV-2 spike pseudotyped lentivirus was added to the cells. 48 hours later, the plate was centrifuged for 5 minutes at 500×g to prevent cell loss. Supernatant was aspirated and 35 μL PBS was added. PBS was carefully aspirated, leaving ˜15 μL of liquid behind. An assay substrate sold under the tradename RENILLA-GLO™ (Promega, Madison, Wisconsin, United States of America) was added to the Assay Buffer at a 1:100 dilution, then 15 μL of the Substrate:Buffer was added to each well of a 384-well plate. The bioluminescence was recorded by a microplate reader sold under the tradename BIOTEK CYTATION™ 5 (BTI Holdings, Winooski, Vermont, United States of America) with an exposure of 200 milliseconds. The infectivity was calculated by the relative luminescence intensity: wells infected with SARS-CoV-2 pseudovirus only were normalized as 100%.

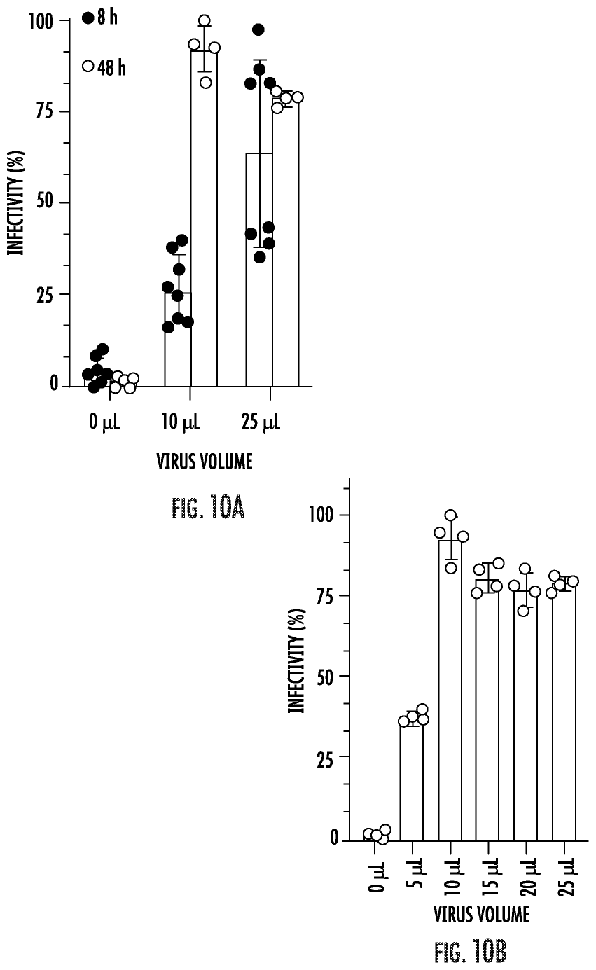

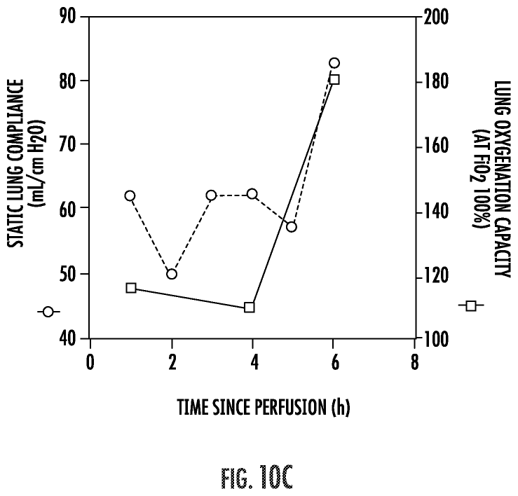

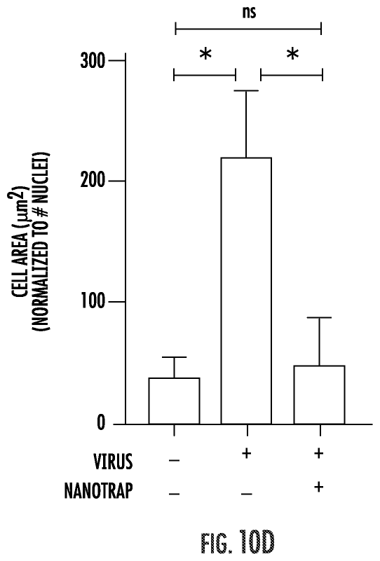

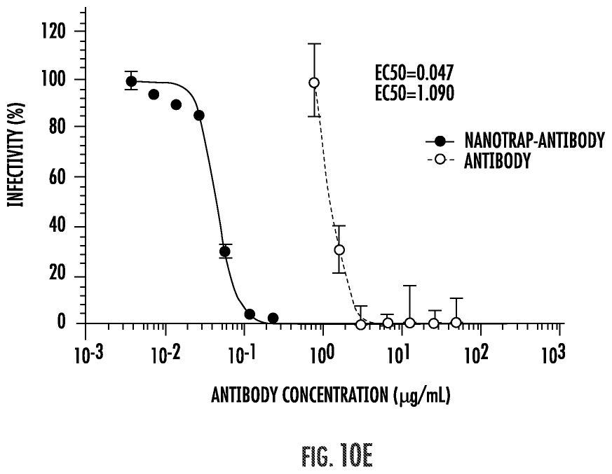

The following equation was used to calculate the antibody mass concentration in FIG. 10E:

$$Antibody mass concentration = \frac{Mass\_Antibody}{Volume} = \frac{n \cdot M}{V} = \frac{\frac{( \# \frac{Ab}{particle} ) \cdot ( \#particle )}{{N}_{A}} \cdot M}{V} = \frac{( \# \frac{Ab}{particle} ) \cdot ( \# \frac{particle}{ml} ) \cdot M}{{N}_{A}} \qquad ( Equation 1 )$$

n: amount of Nanotaps in moles (mol)
M: molecular weight of antibody=150000 Da
V: volume of Nanotrap solution
#Ab/particle: number of the antibody on nanoparticle that is quantified by standard beads as (2.47±0.21)×10⁴
# particles/mL: particle density or nanoparticle that can be counted by hemocytometer and microscope
N_{A}=Avogadro Number=6.022×10²³ particles mol⁻¹

### Example 14

### Authentic SARS-CoV-2 Neutralizing Assay

All SARS-CoV-2 infections were performed in biosafety level 3 conditions. African green monkey kidney (Vero E6) cells were maintained in DMEM supplemented with 10% FBS and 1% Penicillin-Streptomycin. Functionalized nanoparticles or neutralizing antibodies were serially diluted 2-fold and mixed with 400 PFU of SARS-CoV-2 (nCoV/Washington/1/2020, kindly provided by the National Biocontainment Laboratory, Galveston, Texas, United States of America for one hour at 37° C., then used to infect Vero E6 cells for three days. Cells were fixed with 3.7% formalin and stained with 0.25% crystal violet. Crystal violet stained cells were then quantified by absorbance at (595 nm) with a Tecan m200 microplate reader (Tecan, Mannedorf, Switzerland). Cell survival was calculated by normalizing untreated cells to 100%.

### Example 15

### Discussion of Examples 1-14

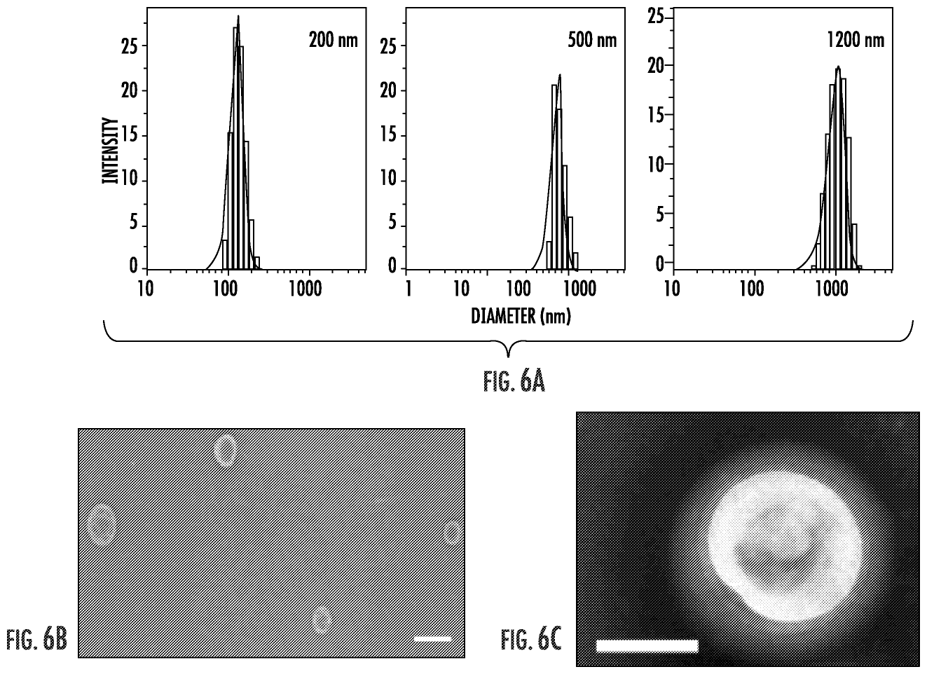

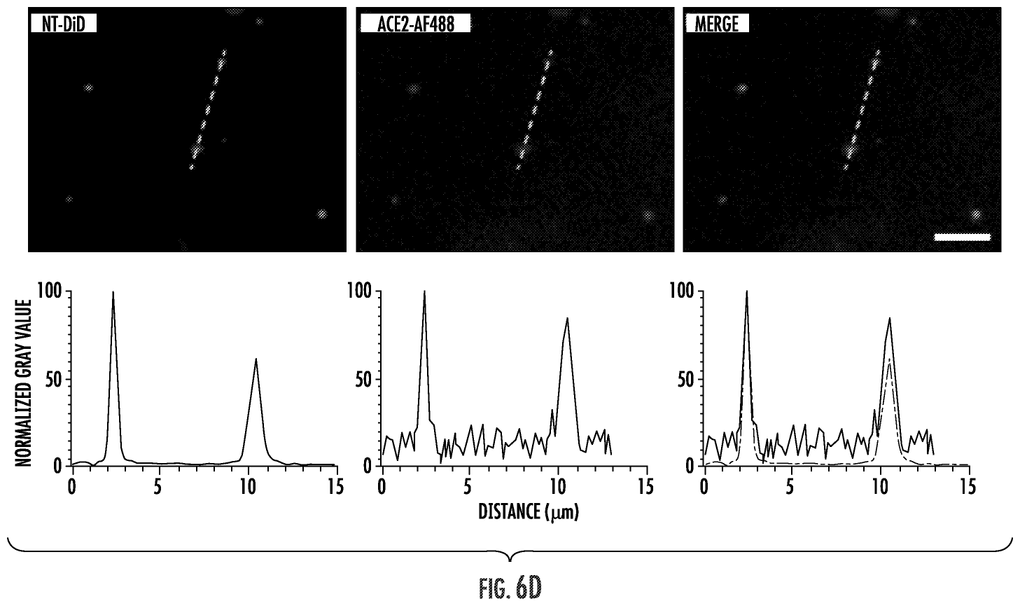

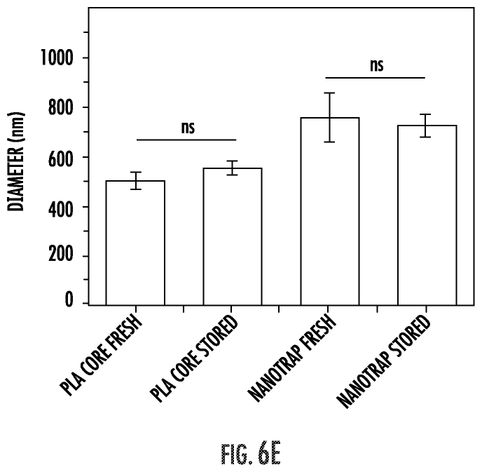

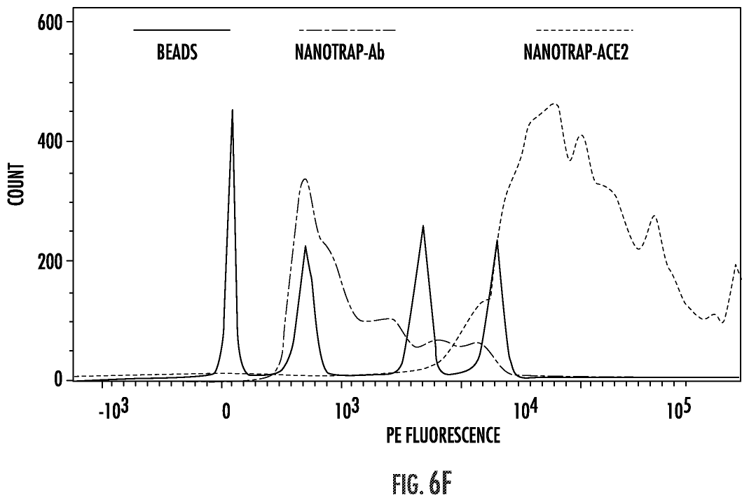

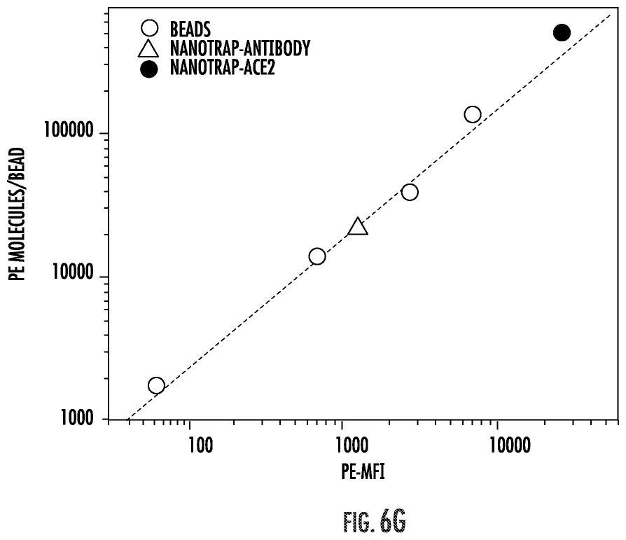

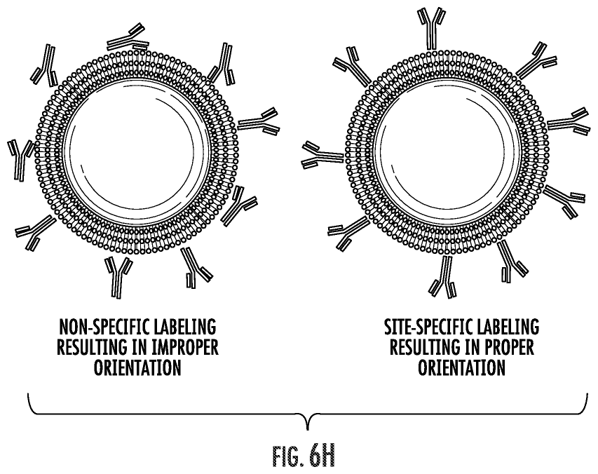

Design, synthesis, and characterization of functionalized nanoparticles: SARS-CoV-2 gains entry into host cells via surface spike proteins that bind to human ACE2 receptors on host cells with very high affinity^{6,26-28}. To inhibit SARS-CoV-2 infection, a family of nano-enabled virus-trapping functionalized nanoparticles, termed “Nanotraps”, were designed to contain and clear SARS-CoV-2. See FIG. 1A. An FDA-approved, biodegradable PLA polymeric core and liposome shell materials were used to synthesize exemplary functionalized nanoparticles. Nanoparticles with different diameters (200, 500, and 1200 nm) were synthesized by varying polymer concentrations (see Examples for details). See FIG. 6A. The solid PLA core acts as a ‘cytoskeleton’ to provide mechanical stability, controlled morphology, biodegradability, and large surface area for nanoscale membrane coating and surface modification. The lipid shell enveloping the PLA core exhibits behavior similar to that of cell membranes. The lipid shell provides a nanoscopic platform and can interact with a wide variety of molecules²⁹⁻³¹ either within the membrane or on the surface^{32,33}. Thus, in one aspect, the presently disclosed subject matter provides for the functionalization of the Nanotrap surface with a molecular bait (a recombinant ACE2 protein or an anti-SARS-CoV-2 neutralizing antibody) and a phagocytosis-inducing ligand (phosphatidylserine). The design of the presently disclosed functionalized nanoparticle is based in part on the assumption that (1) the high-density ACE2 or neutralizing antibodies on the nanoparticles can outcompete low-expression ACE2 on host cells in capturing SARS-CoV-2, thus enabling selective virus containment by the functionalized nanoparticles, and that (2) surface phosphatidylserine ligands on suitably sized functionalized nanoparticles can trigger subsequent phosphatidylserine-mediated phagocytosis by professional phagocytes, such as macrophages, thus enabling viral clearance. See FIG. 1A. The resultant structures were monodispersed and significantly smaller than mammalian cells, yet still large enough to bind several SARS-CoV-2 virions. See FIGS. 1B-1F.

To characterize the functionalized nanoparticles, dynamic light scattering was used to measure the size dispersity of the constituent nanoparticles. Controlling particle size can tune phagocytosis efficacy, and provide reproducible mechanical characteristics and material biocompatibility^{34,35}. The hydrolyzed diameter of the functionalized nanoparticles was measured by dynamic light scattering, which increased with the addition of each molecule. See FIG. 1B. The zeta potential, which reflects the surface charges of the nanoparticles³⁶, was found to change slightly with the addition of each molecule to the nanoparticle surface. See FIG. 1C. Fluorescent labeling and total internal reflection fluorescence microscopy (TIRFM) was used to confirm the presence of the ACE2 moiety on the nanoparticle surface. The PLA polymeric core of exemplary functionalized nanoparticles were labelled with a lipophilic carbocyanine dye: 1,1′-dioctadecyl-3,3,3′,3′-tetramethylindodicarbocyanine, 4-chlorobenzenesulfonate salt (DiD, red). The ACE-2-functionalized nanoparticles (i.e., Nanotrap-ACE2) were further stained with an anti-ACE2 antibody labelled with Alexa fluor-488 dye (AF488, green). The TIRFM images showed excellent co-localization between the nanoparticle core and surface ACE2 at the single particle level, confirmed by line scans of the fluorescent channels corresponding to each component. See FIG. 1D. These results demonstrated that the presently disclosed nanoparticles were successfully functionalized with recombinant ACE2 protein. Also, scanning electron microscopy (SEM) was used to image the functionalized nanoparticles at the sub-nanometer level. SEM images showed that the functionalized nanoparticles were spherical and well-dispersed. See FIG. 6B. The functionalized nanoparticles appeared slightly crenellated in the SEM images. Without being bound to any one theory, this observation is believed to be the result of the lipid layer shrinking due to the drying sample preparation procedure before imaging. See FIG. 1E. As expected, after incubation with the pseudotyped SARS-CoV-2 for 1 hour at 37° C., the functionalized nanoparticles effectively captured the virus as evidenced by single virions clearly visualized on the surface of a nanoparticle; no freestanding virions were observed outside of the functionalized nanoparticle. See FIG. 1F. The representative TEM image clearly shows the core-shell structure of the functionalized nanoparticle. See FIG. 6C. Of note, functionalized nanoparticles that were lyophilized, sealed, and stored at −20° C. for six months demonstrated no change in surface integrity (see FIG. 6D) or size (see FIG. 6E), indicating excellent stability over time.

The surface molecular densities of the two exemplary functionalized nanoparticles were measured by flow cytometry: the ACE2-functionalized nanoparticle (Nanotrap-ACE2) has (3.59±0.43)×10⁵ ACE2 molecules per nanoparticle, and the neutralizing antibody-functionalized nanoparticle (Nanotrap-Antibody) has (2.47±0.2)×10⁴ neutralizing antibodies per nanoparticle. See FIGS. 6F and 6G. Without being bound to any one theory, it is believed that the lower concentration of surface antibodies is due to (1) the lower efficiency conjugation method of NHS-esters versus the site-specific, high-affinity (10⁻¹⁴ M)⁶⁴ biotin-streptavidin conjugation used for ACE2 conjugation in aqueous solution, and (2) imperfect antibody orientation leading to blockage of the antibody binding site to virus spike protein. See FIG. 6H. However, this surface density can be adjusted as desired by simply adjusting the molar ratios of DSPE-PEG₂₀₀₀-biotin or DSPE-PEG₂₀₀₀-NHS on the lipid shell for functionalized nanoparticle synthesis.

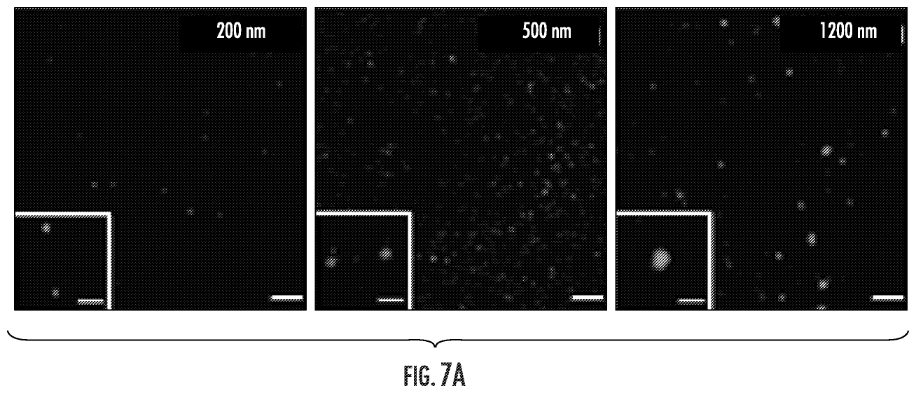

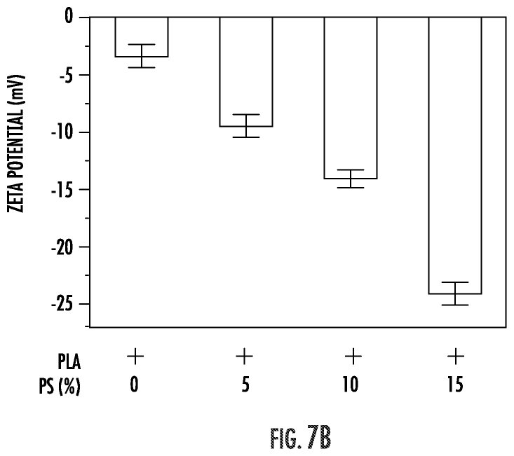

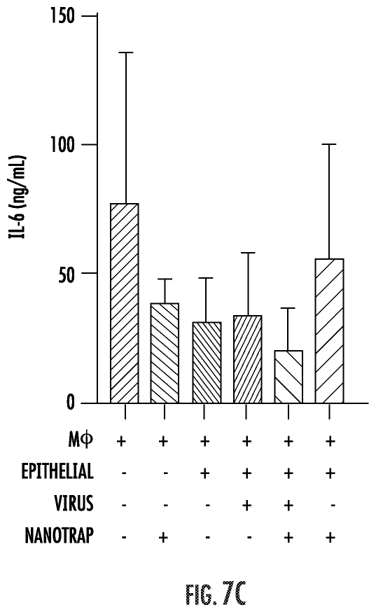

Phagocytosis of functionalized nanoparticles by macrophages: Macrophages are a class of phagocytes that engulf and clear cell debris, pathogens, microbes, cancer cells and other foreign intruders”. Macrophages specialize in the removal of dying or dead cells by recognizing phosphatidylserine on the outer leaflet of the plasma membrane of apoptotic cells¹⁹⁻²¹. Phosphatidylserine coatings have been previously employed to enhance macrophage uptake of liposomal nanoparticles³⁷⁻³⁹. Because phagocytosis by macrophages is dependent on size and surface phosphatidylserine, the optimal size and surface phosphatidylserine density of the functionalized nanoparticles were studied. Nanoparticles labelled with 3,3′-Dioctadecyloxacarbocyanine perchlorate (DiO) fluorescent dye on the PLA polymeric core were synthesized with varying diameters: 200, 500, and 1200 nm. See FIG. 7A. After incubating nanoparticles with differentiated THP-1 (dTHP-1) macrophages⁴⁰ for varying time durations, the size-dependent phagocytosis by dTHP-1 macrophages was examined using flow cytometry. The 500-nm functionalized nanoparticles outperformed the 200-nm and 1200-nm counterparts after 24- and 48-hour incubations, as demonstrated by the percent uptake (see FIG. 2A) and the mean fluorescent intensity of DiO dye (see FIG. 2B) in dTHP-1 macrophages. See also FIGS. 7A and 7B. Accordingly, 500-nm PLA-core functionalized nanoparticles were chosen for all further studies described herein.

Next, the nanoparticles were functionalized with varying surface densities of phosphatidylserine to induce phagocytosis by macrophages. The overall negative charge of the nanoparticles increases as the percentage of phosphatidylserine ratio increases, confirming the presence of phosphatidylserine. See FIG. 7B. Phagocytosis by macrophage was roughly correlated to the percentage of phosphatidylserine. See FIGS. 2C and 2D. The phosphatidylserine-dependent phagocytosis of the functionalized nanoparticles by dTHP-1 macrophages was further demonstrated by three-dimensional lattice light-sheet microscopy images. See FIGS. 2E and 2F. These experiments not only confirmed successful functionalization of the nanoparticles, but also identified the optimal surface density of phosphatidylserine on the nanoparticle surface. Thus, the 500-nm core size and 15% surface phosphatidylserine functionalized nanoparticles were used for the remaining studies to maximize viral capture and macrophage phagocytosis.

Studies were performed to test whether the engulfed functionalized nanoparticles fuse with endosomes or lysosomes within the macrophages. As shown in FIG. 2G, functionalized nanoparticles in various regions in the cell colocalize with either endosomes or lysosomes, suggesting that they will in fact be degraded over time. In addition, the kinetics of 1) the functionalized nanoparticle binding to the virus and 2) the macrophage engulfment of functionalized nanoparticles was studied. See FIG. 2H. The binding between the pseudovirus and the functionalized nanoparticles saturated within an hour (t_{1/2}: 9.4 min), whereas the macrophage engulfment did not reach saturation with the functionalized nanoparticles until 48 h later (t_{1/2}: 1060 min), suggesting that the functionalized nanoparticles bind to virus before being engulfed by the macrophages. Finally, a study was performed to determine if pro-inflammatory cytokine IL-6 was secreted by the macrophages following functionalized nanoparticle engulfment. See FIG. 7C. No significant differences were found amongst the following conditions: 1) Macrophages alone 2) Macrophages and functionalized nanoparticles (Nanotraps) 3) Macrophages and Epithelial cells 4) Macrophages, Epithelial cells, and Virus 5) Macrophages, Epithelial Cells, and functionalized nanoparticles (Nanotraps) and 6) Macrophages, Epithelial Cells, Virus, and functionalized nanoparticles (Nanotraps). Accordingly, these data indicate that the presently disclosed functionalized nanoparticles do not promote a proinflammatory response by the macrophages at the treatment dosage.

Functionalized nanoparticles neutralize SARS-CoV-2 infection in vitro: Multiple types of functionalized nanoparticles were prepared to test their efficacy. First, avi-tagged biotinylated ACE2 was conjugated to the nanoparticle surface via biotin-streptavidin interactions to make functionalized nanoparticles referred to as “Nanotrap-ACE2”. In addition, a functionalized nanoparticle referred to as “Nanotrap-Antibody” was synthesized by conjugating a SARS-CoV-2 neutralizing antibody to the nanoparticle surface via N-hydroxysuccinimide (NHS) esters. Finally, in order to test the specificity of the functionalized nanoparticles, a nanoparticle referred to herein as “Nanotrap-Blank” was prepared without a virus-binding epitope (e.g., an ACE2 protein or SARS-CoV-2 neutralizing antibody).

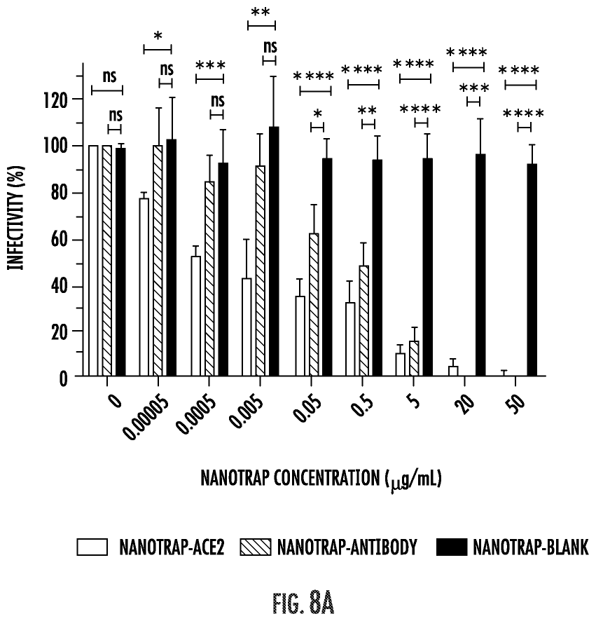

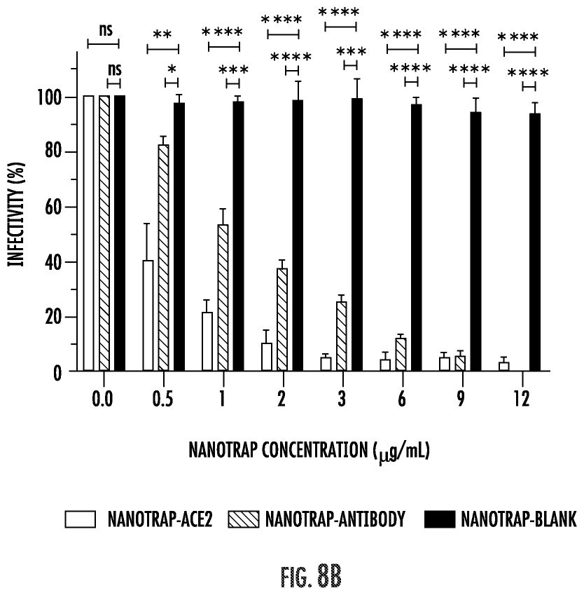

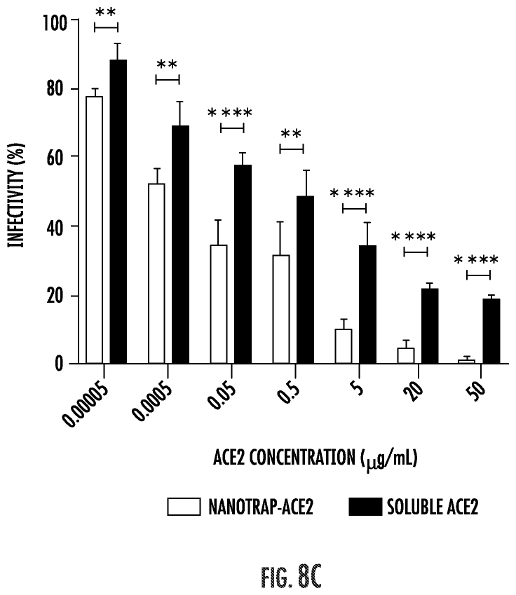

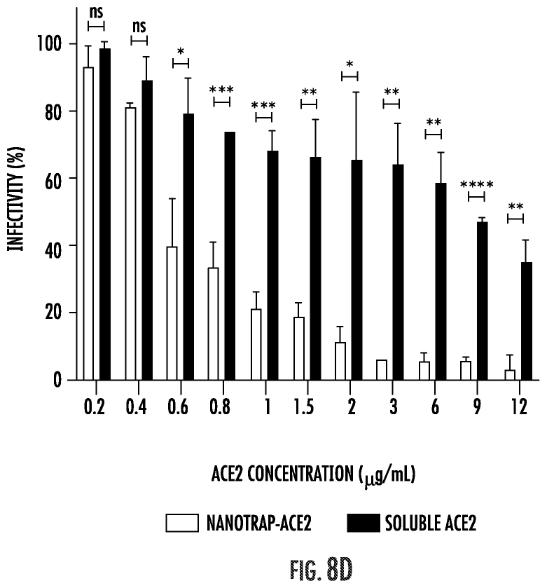

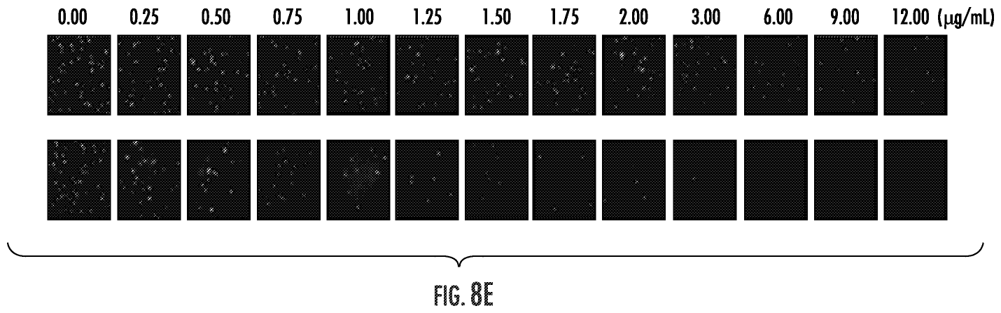

The functionalized nanoparticles were examined to determine if they could effectively capture and contain SARS-CoV-2 in vitro. All three types of nanoparticles were incubated with SARS-CoV-2 spike pseudotyped lentivirus or vesicular stomatitis virus (VSV) for 1 hour before adding to HEK293T-ACE2 cells for 24 hours and 72 hours, respectively. Both Nanotrap-ACE2 and Nanotrap-Antibody completely blocked SARS-CoV-2 pseudovirus infection, while the Nanotrap-Blank did not, indicating both the specificity and functionality of the presently disclosed functionalized nanoparticles. See FIGS. 3A, 8A, 8B, and 8E. Interestingly, despite that neutralizing antibodies have a higher affinity for spike protein (0.07 nM) than that of ACE2 (22 nM)⁶. the Nanotrap-ACE2 blocks SARS-CoV-2 pseudovirus infection more efficiently than the Nanotrap-Antibody. Without being bound to any one theory, this is most likely due to the lower molecular density (see FIG. 2H) and the random orientation (see FIG. 6H) of neutralizing antibody on the functionalized nanoparticles compared to the orientation of the ACE2. Thus, the overall avidity can be lower on the Nanotrap-Antibody than the Nanotrap-ACE2. This can be overcome by using higher-efficiency biotin-streptavidin conjugation rather than the NHS-amine conjugation method used here. In sharp contrast, soluble recombinant ACE2 protein only partially inhibited infection to HEK293T-ACE2 cells with both SARS-CoV-2 spike pseudotyped lentivirus (see FIGS. 3B and 8C) and VSV (see FIGS. 3B and 8D), despite the previous use of soluble ACE2 protein to inhibit SARS-CoV-2 infection^{8,9}.

Macrophages play a role in controlling SARS-CoV-2 infection⁴⁰. Thus, it was further determined whether human macrophages could efficiently engulf and degrade the virus-bound Nanotraps-ACE2 without becoming infected. After incubating SARS-CoV-2 spike pseudotyped VSV with dTHP-1 macrophages for 24 hours, no infection was found in the macrophages (see FIGS. 3C and 3D, comparing “MΦ” with “MΦ +V”). This result demonstrated the feasibility of utilizing macrophages to clear the viral infection. Then, a human lung epithelial cell line A549, which expresses physiological levels of surface ACE2, was infected with SARS-CoV-2 spike pseudotyped VSV in the absence or presence of dTHP-1 macrophages. These data suggest that macrophages significantly reduced the viral infection but could not completely eradicate it. See FIGS. 3C and 3D, comparing “E+V” with “E+ MΦ+V”). Finally, it was determined whether the functionalized nanoparticles triggered phosphatidylserine-mediated phagocytosis by dTIHP-1 macrophages for the clearance of virus. After adding Nanotrap-ACE2 into the co-culture of epithelial cells, macrophages, and SARS-CoV-2 spike pseudotyped VSV, the viral infection was completely inhibited (see FIGS. 3C and 3D, comparing “E+V+NT” and “E+MΦ +V+NT” with “E+MΦ+V”). The incorporation of Nanotrap-ACE2 into the macrophage cell body was observed, indicating successful phagocytosis. See FIG. 3C, “E+MΦ+V+NT” inset.

In brief summary, the in vitro neutralizing experiments demonstrated that the presently disclosed functionalized nanoparticles not only served as a sponge to capture and contain SARS-CoV-2, but also utilized the phagocytosis and sterilization machinery of macrophages to defend the host cells from infection.

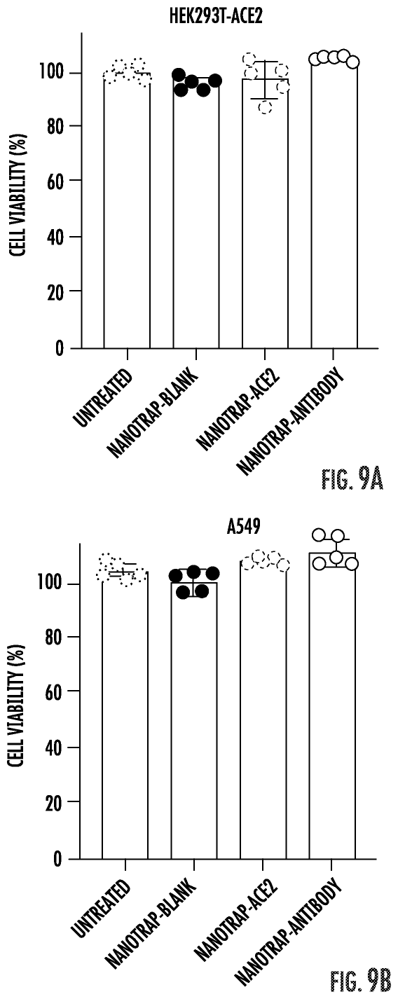

In vivo local delivery to lungs and biosafety profile of functionalized nanoparticles in mice: In order to assess the safety of functionalized nanoparticle treatment, in vitro cytotoxicity was examined using human cell lines. Neither A549 nor HEK293T-ACE2 cells displayed significant cytotoxicity with the addition of Nanotrap-Blank, Nanotrap-ACE2, or Nanotrap-Antibody, as evaluated by a CCK8 cytotoxicity assay. See FIGS. 9A and 9B.

The delivery of functionalized nanoparticles to mouse lungs was examined to evaluate the biosafety of the presently disclosed functionalized nanoparticles in vivo. Immunocompetent mice were intratracheally injected with Nanotrap-ACE2 (labelled with DiD) at a dose of 10 mg/kg. Mice were sacrificed 3 days post-injection. Delivery of functionalized nanoparticle to mouse lungs was confirmed with cryosectioned mouse lung tissues: significant functionalized nanoparticle accumulation and distribution were found in the lung tissues, particularly in regions around bronchioles in the respiratory tracts. No functionalized nanoparticles were found in the lungs of PBS-treated mice. See FIGS. 4A and 4B.

In vivo safety was next analyzed. Hematoxylin and eosin (H&E) staining of major organs including lung, heart, liver, spleen, and kidney showed no histological differences in the functionalized nanoparticle-treated mice when compared to the PBS-treated control group. See FIG. 4C. Furthermore, complete blood counts were performed to evaluate white blood cells (WBCs), red blood cells (RBCs) and platelets (PLTs). The cell counts were similar between functionalized nanoparticle- and PBS-treated groups. See FIG. 4D. Next, comprehensive metabolic panels of mouse blood sera were examined to provide an overall picture of the chemical balance and metabolism. No statistical differences were found between functionalized nanoparticle- and PBS-treated mice for glucose levels, electrolyte and fluid balance, kidney function, or liver function. See FIG. 4E. These results demonstrated the safety of the presently disclosed functionalized nanoparticles when delivered in vivo.

The therapeutic efficacy of functionalized nanoparticles in ex vivo human lungs: The therapeutic efficacy of the presently disclosed functionalized nanoparticles in inhibiting pseudotyped SARS-CoV-2 infection in healthy, non-transplantable human donor lungs was examined using an ex vivo lung perfusion (EVLP) system. See FIG. 5A. EVLP allows a lung to be perfused and ventilated ex vivo after organ retrieval by maintaining lungs at normothermic physiologic conditions and is thus an excellent platform to model lung diseases³⁶.

The infection potential of the SARS-CoV-2 spike pseudotyped lentivirus at different doses over time in primary human lung cells was first tested in vitro, and infection was observed within 8 hours. See FIGS. 10A and 10B. After confirming infection potential, functionalized nanoparticles were tested on an EVLP system with a pair of healthy lungs. Static lung compliance and oxygenation capacity was measured over time. See FIG. 10C. SARS-CoV-2 pseudovirus carrying a luciferase reporter gene was injected into the lingula of left upper lung lobe, and pseudovirus plus Nanotrap-Antibody was injected into the right middle lobe; the right upper lung lobe was used as an untreated control. See FIG. 5A, arrows. Human lung tissue samples were collected after perfusing for 8 hours. Single-cell suspensions were generated, and luciferase expression was determined. See FIG. 5B. The results showed that (1) the pseudovirus infected the lung tissues and (2) the functionalized nanoparticles completely inhibited the viral infection. Furthermore, H&E staining showed significant RBC infiltration in the virus-treated sample, which was not present in the virus plus functionalized nanoparticle-treated region. See FIGS. 5C and 10D.

As the EVLP system maintains lung viability for less than 12 hours, single-cell suspensions of healthy, untreated lung from the right upper lobe was treated in vitro for 48 hours to confirm the functionalized nanoparticles can function for longer term incubations in human tissue. Again, exemplary functionalized nanoparticle Nanotrap-Antibody was able to fully inhibit the virus. See FIG. 5D. In addition, since the EVLP could not be conducted under BSL-3 conditions in order to use authentic SARS-CoV-2, the ability of the functionalized nanoparticles to prevent authentic SARS-CoV-2 from infecting Vero E6 cells, which are highly susceptible to SARS-CoV-2 infection⁴¹, was tested. Exemplary functionalized nanoparticle Nanotrap-Antibody was able to completely inhibit infection of authentic SARS-CoV-2. See FIG. 5E. Of note, the Nanotrap-Antibody outperformed the soluble antibody in the authentic SARS-CoV-2 infection. See FIG. 10E.

Taken together, the EVLP experiments demonstrated that (1) SARS-CoV-2 pseudovirus can infect human lung, and (2) the presently disclosed functionalized nanoparticles can completely block the viral infection, thus paving the way for clinical trials using the presently disclosed functionalized nanoparticles for the inhibition of SARS-CoV-2 infection.

### REFERENCES

All references listed herein including but not limited to all patents, patent applications and publications thereof, scientific journal articles, and database entries are incorporated herein by reference in their entireties to the extent that they supplement, explain, provide a background for, or teach methodology, techniques, and/or compositions employed herein.
1. Dong, E., Du, H. & Gardner, L. An interactive web-based dashboard to track COVID-19 in real time. The Lancet Infectious Diseases 20, 533-534 (2020).
2. Florindo, H. F. et al. Immune-mediated approaches against COVID-19. Nat. Nanotechnol. 15, 630-645 (2020).
3. Shin, M. D. et al. COVID-19 vaccine development and a potential nanomaterial path forward. Nature Nanotechnology 15, 646-655 (2020).
4. Walls, A. C. et al. Structure, Function, and Antigenicity of the SARS-CoV-2 Spike Glycoprotein. Cell 181, 281-292.e6 (2020).
5. Vabret, N. et al. Immunology of COVID-19: Current State of the Science. Immunity 52, 910-941 (2020).
6. Shang, J. et al. Structural basis of receptor recognition by SARS-CoV-2. Nature 581, 221-224 (2020).
7. Shang, J. et al. Cell entry mechanisms of SARS-CoV-2. Proc. Natd. Acad. Sci. U.S.A. 117, (2020).
8. Lei, C. et al. Neutralization of SARS-CoV-2 spike pseudotyped virus by recombinant ACE2-Ig. Nat. Commun. 11, (2020).
9. Monteil, V. et al. Inhibition of SARS-CoV-2 Infections in Engineered Human Tissues Using Clinical-Grade Soluble Human ACE2. Cell 181, 905-913.e7 (2020).
10. Chi, X. et al. A neutralizing human antibody binds to the N-terminal domain of the Spike protein of SARS-CoV-2. Science (80-.). 369, 650-655 (2020).
11. Rogers, T. F. et al. Isolation of potent SARS-CoV-2 neutralizing antibodies and protection from disease in a small animal model. Science (80-.). 369, 956-963 (2020).
12. Shi, R. et al. A human neutralizing antibody targets the receptor-binding site of SARS-CoV-2. Nature 584, 120-124 (2020).
13. Cao, Y. et al. Potent Neutralizing Antibodies against SARS-CoV-2 Identified by High-Throughput Single-Cell Sequencing of Convalescent Patients' B Cells. Cell 182, 73-84.e16 (2020).
14. Chen, X. et al. Human monoclonal antibodies block the binding of SARS-CoV-2 spike protein to angiotensin converting enzyme 2 receptor. Cellular and Molecular Immunology 17, 647-649 (2020).
15. Chen, P. et al. SARS-CoV-2 Neutralizing Antibody LY-CoV555 in Outpatients with Covid-19. N. Engl. J. Med. (2020). doi:10.1056/nejmoa2029849
16. Daassi, D., Mahoney, K. M. & Freeman, G. J. The importance of exosomal PDL1 in tumour immune evasion. Nature Reviews Immunology 20, 209-215 (2020).
17. Gordon, S. Phagocytosis: An Immunobiologic Process. Immunity 44, 463-475 (2016).
18. Liao, M. et al. Single-cell landscape of bronchoalveolar immune cells in patients with COVID-19. Nat. Med. 26, 842-844 (2020).
19. Fadok, V. A., Bratton, D. L., Frasch, S. C., Warner, M. L. & Henson, P. M. The role of phosphatidylserine in recognition of apoptotic cells by phagocytes. Cell Death and Differentiation 5, 551-562 (1998).
20. Wu, Y., Tibrewal, N. & Birge, R. B. Phosphatidylserine recognition by phagocytes: a view to a kill. Trends in Cell Biology 16, 189-197 (2006).
21. Aderem, A. & Underhill, D. M. Mechanisms of phagocytosis in macrophages. Annual Review of Immunology 17, 593-623 (1999).
22. Johnstone, S. A., Masin, D., Mayer, L. & Bally, M. B. Surface-associated serum proteins inhibit the uptake of phosphatidylserine and poly(ethylene glycol) liposomes by mouse macrophages. Biochim. Biophys. Acta—Biomembr. 1513, 25-37 (2001).
23. Shah, N. K., Gupta, S. K., Wang, Z. & Meenach, S. A. Enhancement of macrophage uptake via phosphatidylserine-coated acetalated dextran nanoparticles. J. Drug Deliv. Sci. Technol. 50, 57-65 (2019).
24. Divithotawela, C. et al. Long-term Outcomes of Lung Transplant with Ex Vivo Lung Perfusion. JAMA Surg. 154, 1143-1150 (2019).
25. Cypel, M. et al. Technique for Prolonged Normothermic Ex Vivo Lung Perfusion. J. Hear. Lung Transplant. 27, 1319-1325 (2008).
26. Wang, Q. et al. Structural and Functional Basis of SARS-CoV-2 Entry by Using Human ACE2. Cell 181, 894-904.e9 (2020).
27. Hwang, S. S. et al. MRNA destabilization by BTG1 and BTG2 maintains T cell quiescence. Science (80-.). 367, 1255-1260 (2020).
28. Lan, J. et al. Structure of the SARS-CoV-2 spike receptor-binding domain bound to the ACE2 receptor. Nature 581, 215-220 (2020).
29. Torchilin, V. P. Recent advances with liposomes as pharmaceutical carriers. Nature Reviews Drug Discovery 4, 145-160 (2005).
30. Riley, R. S., June, C. H., Langer, R. & Mitchell, M. J. Delivery technologies for cancer immunotherapy. Nature Reviews Drug Discovery 18, 175-196 (2019).
31. Allen, T. M. & Cullis, P. R. Liposomal drug delivery systems: From concept to clinical applications. Advanced Drug Delivery Reviews 65, 36-48 (2013).
32. Torchilin, V. P. Multifunctional, stimuli-sensitive nanoparticulate systems for drug delivery. Nature Reviews Drug Discovery 13, 813-827 (2014).
33. Suk, J. S., Xu, Q., Kim, N., Hanes, J. & Ensign, L. M. PEGylation as a strategy for improving nanoparticle-based drug and gene delivery. Advanced Drug Delivery Reviews 99, 28-51 (2016).
34. He, C., Hu, Y., Yin, L., Tang, C. & Yin, C. Effects of particle size and surface charge on cellular uptake and biodistribution of polymeric nanoparticles. Biomaterials 31, 3657-3666 (2010).
35. Champion, J. A., Walker, A. & Mitragotri, S. Role of particle size in phagocytosis of polymeric microspheres. Pharm. Res. 25, 1815-1821 (2008).
36. Doane, T. L., Chuang, C. H., Hill, R. J. & Burda, C. Nanoparticle ζ-potentials. Acc. Chem. Res. 45, 317-326 (2012).
37. Toita, R., Kawano, T., Murata, M. & Kang, J. H. Anti-obesity and anti-inflammatory effects of macrophage-targeted interleukin-10-conjugated liposomes in obese mice. Biomaterials 110, 81-88 (2016).
38. Harel-Adar, T. et al. Modulation of cardiac macrophages by phosphatidylserine-presenting liposomes improves infarct repair. Proc. Natd. Acad. Sci. U.S.A 108, 1827-1832 (2011).
39. Han, C. Z. et al. Macrophages redirect phagocytosis by non-professional phagocytes and influence inflammation. Nature 539, 570-574 (2016).
40. Lund, M. E., To, J., O'Brien, B. A. & Donnelly, S. The choice of phorbol 12-myristate 13-acetate differentiation protocol influences the response of THP-1 macrophages to a pro-inflammatory stimulus. J. Immunol. Methods 430, 64-70 (2016).
41. Matsuyama, S. et al. Enhanced isolation of SARS-CoV-2 by TMPRSS2-expressing cells. Proc. Natd. Acad. Sci. U.S.A 117, 7001-7003 (2020).
42. Beigel, J. H. et al. Remdesivir for the Treatment of Covid-19 Final Report. N. Engl. J. Med. (2020). doi:10.1056/nejmoa2007764
43. Wang, Y. et al. Remdesivir in adults with severe COVID-19: a randomised, double-blind, placebo-controlled, multicentre trial. Lancet 395, 1569-1578 (2020).
44. Grein, J. et al. Compassionate Use of Remdesivir for Patients with Severe Covid-19. N. Engl. J. Med. 382, 2327-2336 (2020).
45. Shen, C. et al. Treatment of 5 Critically Ill Patients with COVID-19 with Convalescent Plasma. JAMA—J Am. Med. Assoc. 323, 1582-1589 (2020).
46. Pérez-Cameo, C. & Marín-Lahoz, J. Serosurveys and convalescent plasma in COVID-19. EClinicalMedicine 23, (2020).
47. Chen, G. et al. Exosomal PD-L1 contributes to immunosuppression and is associated with anti-PD-1 response. Nature 560, 382-386 (2018).
48. Mukherjee, A. et al. Lipid-polymer hybrid nanoparticles as a next generation drug delivery platform: State of the art, emerging technologies, and perspectives. International Journal of Nanomedicine 14, 1937-1952 (2019).
49. Janeway, K. M. C. W. C. Janeway's Immunobiology. (Garland Science, 2008).
50. Klichinsky, M. et al. Human chimeric antigen receptor macrophages for cancer immunotherapy. Nat. Biotechnol. 38, 947-953 (2020).
51. Suzuki, T. et al. Generation of human bronchial organoids for SARS-CoV-2 research. Daisuke Okuzaki 4, 2020.05.25.115600 (2020).
52. Han, Y. et al. Identification of Candidate COVID-19 Therapeutics using hPSC-derived Lung Organoids. bioRxiv Prepr. Serv. Biol. (2020). doi: 10.1101/2020.05.05.079095
53. Chandrashekar, A. et al. SARS-CoV-2 infection protects against rechallenge in rhesus macaques. Science (80-.). 369, 812-817 (2020).
54. Yu, J. et al. DNA vaccine protection against SARS-CoV-2 in rhesus macaques. Science (80-.). 369, 806-811 (2020).
55. Rockx, B. et al. Comparative pathogenesis of COVID-19, MERS, and SARS in a nonhuman primate model. Science (80-.). 368, 1012-1015 (2020).
56. Li, S. et al. Ultrasound/Optical Dual-Modality Imaging for Evaluation of Vulnerable Atherosclerotic Plaques with Osteopontin Targeted Nanoparticles. Macromol. Biosci. 20, (2020).
57. Hsu, H. Y., Nicholson, A. C. & Hajjar, D. P. Inhibition of macrophage scavenger receptor activity by tumor necrosis factor-α is transcriptionally and post-transcriptionally regulated. J. Biol. Chem. 271, 7767-7773 (1996).
58. Rosenberg, J. & Huang, J. Visualizing Surface T-Cell Receptor Dynamics Four-Dimensionally Using Lattice Light-Sheet Microscopy. J Vis. Exp. e59914 (2020). doi:10.3791/59914
59. Rosenberg, J., Cao, G., Borja-Prieto, F. & Huang, J. Lattice Light-Sheet Microscopy Multi-dimensional Analyses (LaMDA) of T-Cell Receptor Dynamics Predict T-Cell Signaling States. Cell Syst. 10, 433-444.e5 (2020).
60. Huang, L.-K. & Wang, M.-J. J. Image thresholding by minimizing the measures of fuzziness. Pattern Recognit. 28, 41-51 (1995).
61. Ross, J. T., Nesseler, N., Lee, J. W., Ware, L. B. & Matthay, M. A. The ex vivo human lung: Research value for translational science. JCI Insight 4, (2019).
62. Huang, J., Edwards, L. J., Evavold, B. D. and Zhu, C. Kinetics of MHC-CD8 Interaction at the T Cell Membrane. J. Immunol. 179, 7653-7662 (2007).
63. Liu, S. L., Wang, Z. G., Xie, H. Y., Liu, A. A., Lamb, D. C. and Pang, D. W. Single-Virus Tracking: From Imaging Methodologies to Virological Applications. Chem. Rev. 120, 1936-1979 (2020).
64. Howarth, M., Chinnapen, D. J. F., Gerrow, K., Dorrestein, P. C., Grandy, M. R., Kelleher, N. L., El-Husseini, A. and Ting, A. Y. A monovalent streptavidin with a single femtomolar biotin binding site. Nat. Methods 3, 267-273 (2006).
65. Chan, K. K., Dorosky, D., Sharma, P., Abbasi, S. A., Dye, J. M., Kranz, D. M., Herbert, A. S. and Procko, E. Engineering human ACE2 to optimize binding to the spike protein of SARS coronavirus 2. Science 369, 1261-1265 (2020).
66. Liu et al. Broadly neutralizing antibodies for HIV-1: efficacies, challenges and opportunities. Emerg. Microbes Infect. 9(1), 194-206 (2020).
67. Wang et al. A combination of human broadly neutralizing antibodies against hepatitis B virus HBsAg with distinct epitopes suppresses escape mutations. Cell Host Microbe 28(2), 335-349 (2020).
68. Munjal et al. Advances in developing therapies to combat Zika virus: current knowledge and future perpsectives. Front. Microbiol. 8, 1469 (2017).
69. Du et al. Neutralizing antibodies for the prevention and treatment of COVID-19. Cellular and Molecular Immunology 18, 2293-2306 (2021).
70. Ortega et al. Adhesion to the host cell surface is sufficient to mediate Listeria monoctogenes entry into epithelial cells. Molecular Biology of the Cell 28(22), 2945-2957 (2017).
71. Pizarro-Cerda and Cossart. Bacterial adhesion and entry into host cells. Cell 124(4), P715-727 (2006).
72. Kline et al. Bacterial adhesions in host-microbe interactions. Cell Host & Microbe 5, 580-592 (2009).
73. Motley et al. Monoclonal antibody-based therapies for bacterial infection. Curr. Opp. Infect. Dis. 32(3), 210-216 (2019).

It will be understood that various details of the presently disclosed subject matter may be changed without departing from the scope of the presently disclosed subject matter. Furthermore, the foregoing description is for the purpose of illustration only, and not for the purpose of limitation.

1. A nanoparticle comprising:
(a) a core comprising a biocompatible polymer; and
(b) an outer layer encapsulating said core, wherein the outer layer comprises one or more lipids and/or one or more proteins, and wherein an outer surface of the outer layer comprises:
(c) a pathogen-binding receptor and/or a pathogen-binding antibody or an antigen-binding fragment thereof; and
(d) a phagocyte-specific ligand.

2. The nanoparticle of claim 1, wherein the biocompatible polymer comprises a biodegradable polymer, optionally wherein the biodegradable polymer comprises a polyester, a polyether, or a polyamide, further optionally wherein the biodegradable polymer comprises polylactic acid (PLA), poly(lactic-co-glycolic acid) (PLGA), a PLGA-PLA copolymer, polypyrrole, or a mixture thereof.

3. The nanoparticle of claim 1, wherein the core has a diameter between about 200 and about 1200 nanometers, optionally about 500 nanometers.

4. The nanoparticle of claim 1, wherein (c) comprises a virus-binding receptor and/or a virus-binding antibody or an antigen-binding fragment thereof, optionally a virus-neutralizing antibody or an antigen-binding fragment thereof.

5. The nanoparticle of claim 1, wherein (c) comprises an angiotensin converting enzyme 2 (ACE2) receptor protein, optionally wherein the ACE2 receptor protein is a streptavidin-modified ACE2 receptor protein, the outer layer comprises a biotin-modified lipid, and wherein the streptavidin-modified ACE2 receptor is attached to the surface of the outer layer via biotin-streptavidin non-covalent binding interactions.

6. The nanoparticle of claim 1, wherein (c) comprises a virus-neutralizing antibody or an antigen-binding fragment thereof, optionally wherein the virus-neutralizing antibody or antigen-binding fragment thereof is covalently attached to a lipid in the outer layer via reaction with an active ester-functionalized lipid.

7. The nanoparticle of claim 6, wherein the virus-neutralizing antibody or the antigen-binding fragment thereof is a coronavirus-neutralizing antibody or antigen-binding fragment thereof, optionally a severe acute respiratory syndrome coronavirus 2 (SARS-CoV-2)-neutralizing antibody or antigen-binding fragment thereof.

8. The nanoparticle of claim 7, wherein the virus-neutralizing antibody or the antigen-binding fragment thereof is an antibody or antigen-binding fragment thereof that binds to a SARS-CoV-2 spike protein.

9. The nanoparticle of claim 1, wherein the outer layer comprises one or more phosphatidylserine lipid selected from the group consisting of 1,2-dioleoyl-sn-glycero-3-phospho-L-serine (DOPS), 1,2-distearoyl-sn-glycero-3-phosphatidylserine (DSPS), 1;1-palmitoyl-2-oleoyl-sn-glycero-3-phospho-L-serine (POPS), 1,2-dipalmitoyl-sn-glycero-3-phosphoserine (DPPS), and 1,2-ditetradecanoyl-sn-glycero-3-phospho-L-serine (DMPS); optionally about 15% of the one or more phosphatidylserine lipid; and wherein the phagocyte-specific ligand (d) comprises a phosphoserine moiety from the one or more phosphatidylserine lipid.

10. The nanoparticle of claim 1, wherein the outer layer comprises one or more of DOPS, DSPS, POPS, DPPS, and DMPS, optionally about 15% of the one or more DOPS, DSPS, POPS, DPPS, and DMPS, and wherein the phagocyte-specific ligand (d) comprises a moiety targeting a phagocytic cell, optionally wherein the phagocytic cell is selected from the group consisting of a macrophage, a dendritic cell, a neutrophil, a monocyte, and a mast cell.

11. The nanoparticle of claim 1, wherein the outer layer comprises one or more of the group consisting of 1,2-distearoyl-sn-glycero-3-phosphocholine (DSPC), cholesterol, and a poly(ethylene glycol) (PEG)-modified lipid, optionally 1,2-distearoyl-sn-glycero-3-phosphoethanolamine (DSPE)-mPEG₂₀₀₀.

12. The nanoparticle of claim 1, wherein the core further comprises perfluorooctyl bromide (PFOB).

13. A pharmaceutical formulation comprising a nanoparticle of claim 1 and a pharmaceutically acceptable carrier.

14. A method of treating or preventing a pathogen infection in a subject in need of treatment or prevention thereof, wherein the method comprises administering to said subject a nanoparticle of claim 1.

15. The method of claim 14, wherein the pathogen infection is a viral infection.

16. The method of claim 15, wherein the viral infection is a coronavirus infection, optionally wherein the coronavirus infection is a SARS-CoV-2 infection.

17. The method of claim 14, wherein the administering is performed intranasally.

18. The method of claim 14, wherein the administering is performed orally, intravenously, subcutaneously, intramuscularly, or via ocular administration.

19. The method of claim 14, wherein the subject is a mammal, optionally a human.

20. A nanoparticle of claim 1 for use in treating or preventing a pathogenic infection, optionally a viral infection, further optionally a SARS-CoV-2 infection.

21. A method of treating or preventing a pathogen infection in a subject in need of treatment or prevention thereof, wherein the method comprises administering to said subject a pharmaceutical formulation of claim 13.
